(19) United States
(12) Patent Application Publication
(10) Pub. No.: US 2017/0354603 A1
(43) Pub. Date: Dec. 14, 2017

(54) PHARMACEUTICAL COMPOSITIONS

(71) Applicant: CHARLESTON LABORATORIES, INC., Jupiter, FL (US)

(72) Inventors: Paul BOSSE, Jupiter, FL (US); John HIGGINS, Independence, MO (US); Bernard SCHACHTEL, Jupiter, FL (US); William KOZAREK, Jensen Beach, FL (US)

(21) Appl. No.: 15/618,998

(22) Filed: Jun. 9, 2017

Related U.S. Application Data
(60) Provisional application No. 62/348,688, filed on Jun. 10, 2016.
(60) Provisional application No. 62/398,408, filed on Sep. 22, 2016.
(60) Provisional application No. 62/447,745, filed on Jan. 18, 2017.

(51) Int. Cl. A61K 9/24; A61K 31/5415; A61K 31/485; A61K 9/00; A61K 9/20

(52) U.S. Cl. CPC A61K 9/209; A61K 9/0053; A61K 9/2054; A61K 31/485; A61K 9/2018; A61K 9/2059; A61K 9/2013; A61K 31/5415

(57) ABSTRACT

Pharmaceutical compositions are provided, which comprise effective amounts of an opioid analgesic such as oxycodone, and an antiemetic, such as promethazine, to treat a subject for conditions, including for reducing or eliminating an adverse effect associated with the opioid analgesic.

### CROSS-REFERENCE

The present application claims the benefit of U.S. Provisional Application Ser. No. 62/348,688, filed Jun. 10, 2016; U.S. Provisional Application Ser. No. 62/398,408, filed Sep. 22, 2016; and U.S. Provisional Application Ser. No. 62/447,745, filed Jan. 18, 2017. the contents of each being hereby incorporated by reference in their entirety.

### BACKGROUND

Available pain medications may have adverse effects, such as nausea, vomiting, and skin rashes and sedation. As a result of such adverse effects, many subjects are unable to tolerate recommended dosages needed for effective pain relief because of adverse effects. Accordingly, there remains a need for effective therapeutics with reduced adverse effects.

### INCORPORATION BY REFERENCE

All publications and patent applications mentioned in this specification are herein incorporated by reference to the same extent as if each individual publication or patent application was specifically and individually indicated to be incorporated by reference.

### BRIEF SUMMARY

The present disclosure provides a solid oral pharmaceutical composition that comprises: a first matrix, wherein the first matrix comprises: about 0.5 mg to 30 mg of an opioid analgesic, about 1 mg to 15 mg of croscarmellose sodium, about 1 mg to 20 mg of hydroxypropyl methylcellulose, about 10 mg to 100 mg of mannitol, about 50 mg to 200 mg of silicified microcrystalline cellulose, about 0.1 mg to 5 mg of magnesium stearate, and about 0.1 mg to 5 mg of stearic acid; and a second matrix, wherein the second matrix comprises: about 5 mg to 30 mg of an antiemetic, about 100 mg to 250 mg of silicified microcrystalline cellulose, about 5 mg to 25 mg of croscarmellose sodium, and about 0.1 mg to 5 mg of magnesium stearate. In some instances, the opioid analgesic is selected from the group consisting of oxycodone, hydrocodone, tramadol, hydromorphone, alfentanil, buprenorphine, butorphanol, codeine, codeine, dezocine, diamorphine, fentanyl, levacetylmethadol, levorphanol, meperidine, methadone, morphine, nalbuphine, oxymorphone, pentazocine, propoxyphene, remifentanil, sufentanil, a pharmaceutically acceptable salt thereof, and any combination thereof. In some instances, the opioid analgesic is oxycodone or a pharmaceutically acceptable salt thereof. In some instances, the opioid analgesic is oxycodone hydrochloride. In some instances, the opioid analgesic is present in an amount of about 2.5 mg, about 5 mg, about 7.5 mg, about 10 mg, about 15 mg, or about 20 mg. In some instances, the antiemetic is promethazine or a pharmaceutically acceptable salt thereof. In some instances, the antiemetic is promethazine hydrochloride. In some instances, the antiemetic is present in an amount of about 12.5 to 25 mg. In some instances, in the first matrix are the opioid analgesic that is oxycodone hydrochloride and is present in an amount of about 5 mg, the croscarmellose sodium present in an amount of about 6.7 mg, the hydroxypropyl methylcellulose present in an amount of about 5.6 mg, the mannitol present in an amount of about 60.2 mg, the silicified microcrystalline cellulose present in an amount of about 120.5 mg, the magnesium stearate present in an amount of about 1 mg, and the stearic acid present in an amount of about 1 mg. In some instances, the first matrix further comprises about 5 to 50 mg of starch. In some instances, in the first matrix are the opioid analgesic that is oxycodone hydrochloride and is present in an amount of about 5 mg, the starch present in an amount of about 20 mg, the croscarmellose sodium present in an amount of about 6.7 mg, the hydroxypropyl methylcellulose present in an amount of about 10.3 mg, the mannitol present in an amount of about 38.7 mg, the silicified microcrystalline cellulose present in an amount of about 117.3 mg, the magnesium stearate present in an amount of about 1 mg, and the stearic acid present in an amount of about 1 mg. In some instances, in the second matrix are the antiemetic that is promethazine hydrochloride and is present in an amount of about 12.5 mg, the silicified microcrystalline cellulose present in an amount of about 121.5 mg, the croscarmellose sodium present in an amount of about 15 mg, and the magnesium stearate present in an amount of about 1 mg. In some instances, as measured by USP Apparatus 1 with a basket rotating at 50 rpm, the solid oral pharmaceutical composition has one or more of the following release rates following contact with a dissolution medium of 900 mL of 0.01 N HCl at about 37° C.: about 20-50% of the opioid analgesic is released within about 5 minutes; about 30-60% of the opioid analgesic is released within about 10 minutes; about 40-70% of the opioid analgesic is released within about 15 minutes; about 60-90% of the opioid analgesic is released within about 30 minutes; about 70-100% of the opioid analgesic is released within about 60 minutes; or about 80-100% of the opioid analgesic is released within about 90 minutes. In some instances, as measured by USP Apparatus 1 with a basket rotating at 50 rpm, the solid oral pharmaceutical composition has one or more of the following release rates following contact with a dissolution medium of 900 mL of 0.01 N HCl at about 37° C.; about 30-37% of the opioid analgesic is released within about 5 minutes; about 42-51% of the opioid analgesic is released within about 10 minutes; about 52-60% of the opioid analgesic is released within about 15 minutes; about 70-74% of the opioid analgesic is released within about 30 minutes; about 83-85% of the opioid analgesic is released within about 60 minutes; or about 87-90% of the opioid analgesic is released within about 90 minutes. In some instances, as measured by USP Apparatus 1 with a basket rotating at 50 rpm, the solid oral pharmaceutical composition has one or more of the following release rates following contact with a dissolution medium of 900 mL of 0.01 N HCl at about 37° C.: about 25-45% of the antiemetic is released within about 5 minutes; about 35-60% of the antiemetic is released within about 10 minutes; about 45-70% of the antiemetic is released within about 15 minutes; about 60-90% of the antiemetic is released within about 30 minutes; about 80-100% of the antiemetic is released within about 60 minutes; or about 90-100% of the anti emetic is released within about 90 minutes. In some instances, as measured by USP Apparatus 1 with a basket rotating at 50 rpm, the solid oral pharmaceutical composition has one or more of the following release rates following contact with a dissolution medium of 900 mL of 0.01 N HCl at about 37° C.: about 36% of the antiemetic is released within about 5 minutes; about 47-48% of the antiemetic is released within about 10 minutes; about 56% of the antiemetic is released within about 15 minutes; about 74-75% of the antiemetic is released within about 30 minutes; about 93% of the antiemetic is released within about 60 minutes; or about 100% of the antiemetic is released within about 90 minutes. In some instances, the first matrix is a layer. In some instances, the second matrix is a layer. In some instances, the solid oral pharmaceutical composition provides an effective amount of the opioid analgesic to treat or prevent pain in a subject in need thereof and an effective amount of the antiemetic to reduce or prevent an adverse effect associated with the opioid analgesic in the subject.

The present disclosure also provides a solid oral pharmaceutical composition that comprises: a first matrix, wherein the first matrix comprises: an opioid analgesic, croscarmellose sodium, hydroxypropyl methylcellulose, mannitol, silicified microcrystalline cellulose, magnesium stearate, and stearic acid; and a second matrix, wherein the second matrix comprises: an antiemetic, silicified microcrystalline cellulose, croscarmellose sodium, and magnesium stearate, wherein: when the solid oral pharmaceutical composition is stored at a temperature less than 80° C. for a time period of at least 30 days, about 90% to about 100% of the antiemetic is active as measured by HPLC, and about 80% to about 100% of the opioid analgesic is active for as measured by HPLC. In some instances, the temperature is in a range of about 40 to 75° C. In some instances, the temperature is in a range of about 25 to 60° C. in some instances, the time period is at least 60 days. In some instances, the first matrix further comprises starch. In some instances, the opioid analgesic is selected from the group consisting of oxycodone, hydrocodone, tramadol, hydromorphone, alfentanil, buprenorphine, butorphanol, codeine, codeine, dezocine, diamorphine, fentanyl, levacetylmethadol, levorphanol, meperidine, methadone, morphine, nalbuphine, oxymorphone, pentazocine, propoxyphene, remifentanil, sufentanil, a pharmaceutically acceptable salt thereof, and any combination thereof. In some instances, the opioid analgesic is oxycodone or a pharmaceutically acceptable salt thereof. In some instances, the opioid analgesic is oxycodone hydrochloride, in some instances, the opioid analgesic is present in an amount of about 2.5 mg, about 5 mg, about 7.5 mg, about 10 mg, about 15 mg, or about 20 mg. In some instances, the antiemetic is promethazine or a pharmaceutically acceptable salt thereof. In some instances, the antiemetic is promethazine hydrochloride. In some instances, the antiemetic is present in an amount of about 12.5 to 25 mg. In some instances, the opioid analgesic is oxycodone hydrochloride and is present in an amount of about 5 mg, and the antiemetic is promethazine hydrochloride and is present in an amount of about 12.5 mg. In some instances, the first matrix is a layer. In some instances, the second matrix is a layer. In some instances, the solid oral pharmaceutical composition provides an effective amount of the opioid analgesic to treat or prevent pain in a subject in need thereof and an effective amount of the antiemetic to reduce or prevent an adverse effect associated with the opioid analgesic in the subject.

The present disclosure also provides a solid oral pharmaceutical composition that comprises: a first matrix that comprises an opioid analgesic; and a second matrix that comprises an antiemetic, wherein about 30% to about 60% of the antiemetic is released within about 5 to 15 minutes in a dissolution medium as measured by USP Apparatus 1 with a basket rotating at 50 rpm, wherein the dissolution medium is 900 mL of 0.01 N HCl at about 37° C. In some instances, about 30% to about 50% of the antiemetic is released within about 5 to 10 minutes in the dissolution medium as measured by USP Apparatus 1 with a basket rotating at 50 rpm. In some instances, about 30% to about 40% of the antiemetic is released within about 5 minutes in the dissolution medium as measured by USP Apparatus 1 with a basket rotating at 50 rpm. In some instances, about 70% of the antiemetic is released within about 30 minutes in the dissolution medium as measured by USP Apparatus 1 with a basket rotating at 50 rpm. In some instances, about 90% to about 100% of the antiemetic is released within about 60 to 90 minutes in the dissolution medium as measured by USP Apparatus 1 with a basket rotating at 50 rpm. In some instances, about 30% to about 50% of the opioid analgesic is released within about 5 to 15 minutes in the dissolution medium as measured by USP Apparatus 1 with a basket rotating at 50 rpm. In some instances, about 30% to about 40% of the opioid analgesic is released within about 5 to 10 minutes in the dissolution medium as measured by USP Apparatus 1 with a basket rotating at 50 rpm. In some instances, the first matrix further comprises: croscarmellose sodium, hydroxypropyl methylcellulose, mannitol, silicified microcrystalline cellulose, magnesium stearate, and stearic acid; and the second matrix further comprises: silicified microcrystalline cellulose, croscarmellose sodium, and magnesium stearate. In some instances, the first matrix further comprises starch. In some instances, the opioid analgesic is selected from the group consisting of oxycodone, hydrocodone, tramadol, hydromorphone, alfentanil, buprenorphine, butorphanol, codeine, codeine, dezocine, diamorphine, fentanyl, levacetylmethadol, levorphanol, meperidine, methadone, morphine, nalbuphine, oxymorphone, pentazocine, propoxyphene, remifentanil, sufentanil, a pharmaceutically acceptable salt thereof, or any combination thereof. In some instances, the opioid analgesic is oxycodone or a pharmaceutically acceptable salt thereof. In some instances, the opioid analgesic is oxycodone hydrochloride. In some instances, the opioid analgesic is present in an amount of about 2.5 mg, about 5 mg, about 7.5 mg, about 10 mg, about 15 mg, or about 20 mg. In some instances, the antiemetic is promethazine or a pharmaceutically acceptable salt thereof. In some instances, the antiemetic is promethazine hydrochloride. In some instances, the antiemetic is present in an amount of about 12.5 to 25 mg. In some instances, the opioid analgesic is oxycodone hydrochloride and is present in an amount of about 5 mg, and the antiemetic is promethazine hydrochloride and is present in an amount of about 12.5 mg. In some instances, the first matrix is a layer, in some instances, the second matrix is a layer. In some instances, the solid oral pharmaceutical composition provides an effective amount of the opioid analgesic to treat or prevent pain in a subject in need thereof and an effective amount of the antiemetic to reduce or prevent an adverse effect associated with the opioid analgesic in the subject.

The present disclosure also provides a method for treating or preventing pain, comprising orally administering to a subject in need thereof a solid oral pharmaceutical composition disclosed herein. In some instances, the adverse effect associated with the opioid analgesic is nausea, vomiting, constipation, itching, gastric upset, or a skin rash. In some instances, the adverse effect is nausea or vomiting. In some instances, the adverse effect comprises nausea and vomiting. In some instances, the subject has a reduction in incidence of nausea or vomiting. In some instances, the subject has a reduction in intensity of nausea or vomiting. In some instances, the solid oral pharmaceutical composition is administered at least two times a day. In some instances, the solid oral pharmaceutical composition is administered at least three times a day. In some instances, the solid oral pharmaceutical composition is administered in a dosing interval of about 4 to about 6 hours. In some instances, the subject is a child. In some instances, the subject is an adult. In some instances, the subject is younger than 12 years of age. In some instances, the subject is 6 years of age or older. In some instances, the subject is 12 years of age or older.

The present disclosure also provides a solid oral pharmaceutical composition disclosed herein is for use in a medicament, e.g., a medicament for the treatment or prevention of pain. In some cases, a solid oral pharmaceutical composition disclosed herein is for use in the treatment or prevention of pain. The present disclosure also provides use of a solid oral pharmaceutical composition disclosed herein in the manufacture of a medicament for treatment or prevention of pain.

### BRIEF DESCRIPTION OF THE DRAWINGS

FIG. 1 depicts a plot of oxycodone dissolution rates for the indicated compositions in Example 12.

FIG. 2 depicts a plot of promethazine dissolution rates for the indicated compositions in Example 12.

FIG. 3 depicts a plot of oxycodone and promethazine rates for the indicated compositions in Example 13.

### DETAILED DESCRIPTION

### Definitions

The term “about” means the referenced numeric indication plus or minus 15% of that referenced numeric indication.

The term “subject” as used herein refers to a mammal (e.g., a human, mouse, rat, guinea pig, dog, cat, horse, cow, pig, or non-human primate, such as a monkey, chimpanzee or baboon). In a particular instance the subject is a human subject.

The term “controlled-release” or “controlled release” refers to release of at least one pharmaceutically active agent in a formulation or component of a formulation (e.g., a layer in a tablet, a particulate in a multi-particulate composition, etc.) at a time later than immediately after contact with a dissolution fluid or administration to a subject. A controlled-release formulation or component of a formulation has a slower release of the at least one pharmaceutically active agent than a pharmaceutically active agent in an immediate-release formulation or component of a formulation. In some instances, a controlled-release formulation begins its release and continues that release over an extended period of time. In some instances, the rate of release in a controlled-release formulation is constant over time. In some instances, the rate of release in a controlled-release formulation increases or decreases over time. In some instances, the rate of release in a controlled-release formulation is pulsed, continuous or intermittent, and the like.

The term “immediate-release” or “immediate release” refers to the release of at least one pharmaceutically active agent in a formulation or component of a formulation (e.g., a layer in a tablet, a particulate in a multi-particulate composition, etc.) that is rapid after contact with a dissolution fluid or administration to a subject. An immediate-release formulation or component of a formulation has a faster release of the at least one pharmaceutically active agent than a pharmaceutically active agent in a controlled-release formulation or component of a formulation. In some instances, an immediate-release formulation or component of a formulation has a faster release of the at least one pharmaceutically active agent than a pharmaceutically active agent in a standard or unmodified formulation or component of a formulation.

An “effective amount” when used in connection with an opioid analgesic agent is an amount that is effective for treating or preventing pain.

An “effective amount” when used in connection with an antiemetic agent is an amount that is effective for preventing, reducing or eliminating one or more adverse effects associated with the opioid analgesic in a subject in need thereof.

The term “pain” as used herein to all types of pain, in particular moderate to severe pain. Pain includes neuropathic pain, post-operative pain, chronic lower back pain, cluster headaches, herpes neuralgia, phantom limb pain, central pain, dental pain, neuropathic pain, visceral pain, surgical pain, bone injury pain, pain during labor and delivery, pain resulting from burns, post partum pain, migraine, angina pain, genitourinary tract-related pain including cystitis and nociceptive pain. In some instances, the pain is chronic or acute (“chronic pain” or “acute pain”). The term “post-operative pain” as used herein refers to a subject's pain after surgery.

Provided herein are pharmaceutical compositions comprising multiple pharmaceutically active agents that are useful as therapeutics that alleviate, abate or eliminate one or more conditions in a subject in need thereof, as further described herein below. In some instances, the pharmaceutical compositions described herein comprise one active agent that provides pain relief and a second active agent that reduce or prevent adverse effects associated with the first active agent. In some instances, the combination of active agents is in a single formulation.

The present disclosure provides a solid oral pharmaceutical composition that comprises: a first matrix, wherein the first matrix comprises: about 0.5 mg to 30 mg of an opioid analgesic, about 1 mg to 30 mg (e.g., about 1-15 mg) of croscarmellose sodium, about 1 mg to 40 mg (e.g., about 1-20 mg) of hydroxypropyl methylcellulose, about 5 mg to 150 mg (e.g., about 5-50 mg, about 10-100 mg) of mannitol, about 10 mg to 300 mg (e.g., about 50-200 mg, about 50-150 mg) of silicified microcrystalline cellulose, about 0.1 mg to 15 mg of magnesium stearate (e.g., about 0.1-10 mg, about 0.1-5 mg), and about 0.1 mg to 15 mg of stearic acid (e.g., about 0.1-10 mg, about 0.1-5 mg); and a second matrix, wherein the second matrix comprises: about 5 mg to 30 mg of an antiemetic, about 10 mg to 300 mg of silicified microcrystalline cellulose (e.g., about 100-250 mg, about 50-200 mg, about 50-150 mg), about 5 mg to 50 mg of croscarmellose sodium (e.g., about 5-25 mg), and about 0.1 mg to 15 mg of magnesium stearate (e.g., about 0.1-10 mg, about 0.1-5 mg). In some instances, the opioid analgesic is selected from the group consisting of oxycodone, hydrocodone, tramadol, hydromorphone, alfentanil, buprenorphine, butorphanol, codeine, codeine, dezocine, diamorphine, fentanyl, levacetylmethadol, levorphanol, meperidine, methadone, morphine, nalbuphine, oxymorphone, pentazocine, propoxyphene, remifentanil, sufentanil, a pharmaceutically acceptable salt thereof, and any combination thereof. In some instances, the opioid analgesic is oxycodone or a pharmaceutically acceptable salt thereof. In some instances, the opioid analgesic is oxycodone hydrochloride. In some instances, the opioid analgesic is present in an amount of about 2.5 mg, about 5 mg, about 7.5 mg, about 10 mg, about 15 mg, or about 20 mg. In some instances, the antiemetic is promethazine or a pharmaceutically acceptable salt thereof. In some instances, the anti emetic is promethazine hydrochloride. In some instances, the antiemetic is present in an amount of about 12.5 to 25 mg, about 5 to 25 mg, or about 5 to 12.5 mg. In some instances, present in the first matrix are about 5 mg of oxycodone hydrochloride, about 6.7 mg of croscarmellose sodium, about 5.6 mg of hydroxypropyl methylcellulose, about 60.2 mg of mannitol, about 120.5 mg of silicified microcrystalline cellulose, about 1 mg of magnesium stearate, and about 1 mg of stearic acid. In sonic instances, the first matrix further comprises about 5 to 50 mg of starch, e.g., partially pregelatinized maize starch. In some instances, present in the first matrix are about 5 mg of oxycodone hydrochloride, about 20 mg of starch, about 6.7 mg of croscarmellose sodium, about 10.3 mg of hydroxypropyl methylcellulose, about 38.7 mg of mannitol, about 117.3 mg of silicified microcrystalline cellulose, about 1 mg of magnesium stearate, and about 1 mg of stearic acid. In some instances, present in the second matrix are about 12.5 mg of the anti emetic, about 121.5 mg of silicified microcrystalline cellulose, about 15 mg of croscarmellose sodium, and about 1 mg of magnesium stearate. In some instances, as measured by USP Apparatus 1 with a basket rotating at 50 rpm, the solid oral pharmaceutical composition has one or more of the following release rates following contact with a dissolution medium of 900 mL of 0.01 N HCl at about 37° C.: about 20-50% of the opioid analgesic is released within about 5 minutes; about 30-60% of the opioid analgesic is released within about 10 minutes; about 40-70% of the opioid analgesic is released within about 15 minutes; about 60-90% of the opioid analgesic is released within about 30 minutes; about 70-100% of the opioid analgesic is released within about 60 minutes; or about 80-100% of the opioid analgesic is released within about 90 minutes. In some instances, as measured by USP Apparatus 1 with a basket rotating at 50 rpm, the solid oral pharmaceutical composition has one or more of the following release rates following contact with a dissolution medium of 900 mL of 0.01 N HCl at about 37° C.: about 30-37% of the opioid analgesic is released within about 5 minutes; about 42-51% of the opioid analgesic is released within about 10 minutes; about 52-60% of the opioid analgesic is released within about 15 minutes; about 70-74% of the opioid analgesic is released within about 30 minutes; about 83-85% of the opioid analgesic is released within about 60 minutes; or about 87-90% of the opioid analgesic is released within about 90 minutes. In some instances, as measured by USP Apparatus 1 with a basket rotating at 50 rpm, the solid oral pharmaceutical composition has one or more of the following release rates following contact with a dissolution medium of 900 mL of 0.01 N HCl at about 37° C.: about 25-45% of the antiemetic is released within about 5 minutes; about 35-60% of the antiemetic is released within about 10 minutes; about 45-70% of the antiemetic is released within about 15 minutes; about 60-90% of the antiemetic is released within about 30 minutes; about 80-100% of the antiemetic is released within about 60 minutes; or about 90-100% of the antiemetic is released within about 90 minutes. In some instances, as measured by USP Apparatus 1 with a basket rotating at 50 rpm, the solid oral pharmaceutical composition has one or more of the following release rates following contact with a dissolution medium of 900 mL of 0.01 N HCl at about 37° C.: about 36% of the antiemetic is released within about 5 minutes; about 47-48% of the antiemetic is released within about 10 minutes; about 56% of the antiemetic is released within about 15 minutes; about 74-75% of the antiemetic is released within about 30 minutes; about 93% of the antiemetic is released within about 60 minutes; or about 100% of the antiemetic is released within about 90 minutes. In some instances, the first matrix is a layer. In some instances, the second matrix is a layer. In some instances, the solid oral pharmaceutical composition provides an effective amount of the opioid analgesic to treat or prevent pain in a subject in need thereof and an effective amount of the antiemetic to reduce or prevent an adverse effect associated with the opioid analgesic in the subject.

The present disclosure also provides a solid oral pharmaceutical composition that comprises: a first matrix, wherein the first matrix comprises: an opioid analgesic, croscarmellose sodium, hydroxypropyl methylcellulose, mannitol, silicified microcrystalline cellulose, magnesium stearate, and stearic acid; and a second matrix, wherein the second matrix comprises: an antiemetic, silicified microcrystalline cellulose, croscarmellose sodium, and magnesium stearate, wherein: when the solid oral pharmaceutical composition is stored at a temperature less than 80° C. for a time period of at least 30 days, about 90% to about 100% of the antiemetic is active as measured by HPLC, and about 80% to about 100% of the opioid analgesic is active for as measured by HPLC. In some instances, the temperature is in a range of about 40 to 75° C. In some instances, the temperature is in a range of about 25 to 60° C. In some instances, the time period is at least 60 days. In some instances, the first matrix further comprises starch, e.g., partially pregelatinized maize starch. In some instances, the opioid analgesic is selected from the group consisting of oxycodone, hydrocodone, tramadol, hydromorphone, alfentanil, buprenorphine, butorphanol, codeine, codeine, dezocine, diamorphine, fentanyl, levacetylmethadol, levorphanol, meperidine, methadone, morphine, nalbuphine, oxymorphone, pentazocine, propoxyphene, remifentanil,sufentanil, a pharmaceutically acceptable salt thereof, and any combination thereof. In some instances, the opioid analgesic is oxycodone or a pharmaceutically acceptable salt thereof. In some instances, the opioid analgesic is oxycodone hydrochloride. In some instances, the opioid analgesic is present in an amount of about 2.5 mg, about 5 mg, about 7.5 mg, about 10 mg, about 15 mg, or about 20 mg. In some instances, the antiemetic is promethazine or a pharmaceutically acceptable salt thereof. In some instances, the antiemetic is promethazine hydrochloride. In some instances, the antiemetic is present in an amount of about 12.5 to 25 mg, about 5 to 25 mg, or about 5 to 12.5 mg. In some instances, the opioid analgesic is oxycodone hydrochloride and is present in an amount of about 5 mg, and the antiemetic is promethazine hydrochloride and is present in an amount of about 12.5 mg. In some instances, the first matrix is a layer. In some instances, the second matrix is a layer. In some instances, the solid oral pharmaceutical composition provides an effective amount of the opioid analgesic to treat or prevent pain in a subject in need thereof and an effective amount of the antiemetic to reduce or prevent an adverse effect associated with the opioid analgesic in the subject.

The present disclosure also provides a solid oral pharmaceutical composition that comprises: a first matrix that comprises an opioid analgesic; and a second matrix that comprises an antiemetic, wherein about 30% to about 60% of the antiemetic is released within about 5 to 15 minutes in a dissolution medium as measured by USP Apparatus 1 with a basket rotating at 50 rpm, wherein the dissolution medium is 900 mL of 0.01 N HCl at about 37° C. In some instances, about 30% to about 50° io of the antiemetic is released within about 5 to 10 minutes in the dissolution medium as measured by USP Apparatus 1 with a basket rotating at 50 rpm. In some instances, about 30% to about 40% of the antiemetic is released within about 5 minutes in the dissolution medium as measured by USP Apparatus 1 with a basket rotating at 50 rpm. In some instances, about 70% of the antiemetic is released within about 30 minutes in the dissolution medium as measured by USP Apparatus 1 with a basket rotating at 50 rpm. In some instances, about 90% to about 100% of the antiemetic is released within about 60 to 90 minutes in the dissolution medium as measured by USP Apparatus 1 with a basket rotating at 50 rpm. In some instances, about 30% to about 50% of the opioid analgesic is released within about 5 to 15 minutes in the dissolution medium as measured by USP Apparatus 1 with a basket rotating at 50 rpm. In some instances, about 30% to about 40% of the opioid analgesic is released within about 5 to 10 minutes in the dissolution medium as measured by USP Apparatus 1 with a basket rotating at 50 rpm. In some instances, the first matrix further comprises: croscarmellose sodium, hydroxypropyl methylcellulose, mannitol, silicified microcrystalline cellulose, magnesium stearate, and stearic acid; and the second matrix further comprises: silicified microcrystalline cellulose, croscarmellose sodium, and magnesium stearate. In some instances, the first matrix further comprises starch, e.g., partially pregelatinized maize starch. In some instances, the opioid analgesic is selected from the group consisting of oxycodone, hydrocodone, tramadol, hydromorphone, alfentanil, buprenorphine, butorphanol, codeine, codeine, dezocine, diamorphine, fentanyl, levacetylmethadol, levorphanol, meperidine, methadone, morphine, nalbuphine, oxymorphone, pentazocine, propoxyphene, remifentanil, sufentanil, a pharmaceutically acceptable salt thereof, or any combination thereof. In some instances, the opioid analgesic is oxycodone or a pharmaceutically acceptable salt thereof. In some instances, the opioid analgesic is oxycodone hydrochloride. In some instances, the opioid analgesic is present in an amount of about 2.5 mg, about 5 mg, about 7.5 mg, about 10 mg, about 15 mg, or about 20 mg. In some instances, the antiemetic is promethazine or a pharmaceutically acceptable salt thereof. In some instances, the antiemetic is promethazine hydrochloride. In some instances, the antiemetic is present in an amount of about 12.5 to 25 mg, about 5 to 25 mg, or about 5 to 12.5 mg. In some instances, the opioid analgesic is oxycodone hydrochloride and is present in an amount of about 5 mg, and the antiemetic is promethazine hydrochloride and is present in an amount of about 12.5 mg. In some instances, the first matrix is a layer. In some instances, the second matrix is a layer. In some instances, the solid oral pharmaceutical composition provides an effective amount of the opioid analgesic to treat or prevent pain in a subject in need thereof and an effective amount of the antiemetic to reduce or prevent an adverse effect associated with the opioid analgesic in the subject.

The present disclosure also provides a method of treating or preventing pain, comprising orally administering to a subject in need thereof a solid oral pharmaceutical composition disclosed herein. In some instances, the adverse effect associated with the opioid analgesic is nausea, vomiting, constipation, itching, gastric upset, or a skin rash. In some instances, the adverse effect is nausea or vomiting. In some instances, the adverse effect comprises nausea and vomiting. In some instances, the subject has a reduction in incidence of nausea or vomiting. In some instances, the subject has a reduction in intensity of nausea or vomiting. In some instances, the solid oral pharmaceutical composition is administered at least two times a day. In some instances, the solid oral pharmaceutical composition is administered at least three times a day. In some instances, the solid oral pharmaceutical composition is administered in a dosing interval of about 4 to about 6 hours. In some instances, the subject is a child. In some instances, the subject is an adult. In some instances, the subject is younger than 12 years of age. In some instances, the subject is 6 years of age or older. In some instances, the subject is 12 years of age or older.

The present disclosure also provides a solid oral pharmaceutical composition disclosed herein is for use in a medicament, e.g., a medicament for the treatment or prevention of pain. In some cases, a solid oral pharmaceutical composition disclosed herein is for use in the treatment or prevention of pain. The present disclosure also provides use of a solid oral pharmaceutical composition disclosed herein in the manufacture of a medicament for treatment or prevention of pain.

Provided herein are solid oral pharmaceutical compositions for treatment of pain and prevention or reduction of an adverse effect associated with an opioid analgesic, comprising a first matrix, wherein the first matrix comprises about 0.5 mg to 20 mg of an opioid analgesic, wherein the opioid analgesic is oxycodone or a pharmaceutically acceptable salt thereof, about 1 mg to 15 mg of croscarmellose sodium, about 1 mg to 20 mg of hydroxypropyl methylcellulose, about 10 mg to 100 mg of mannitol, about 50 mg to 200 mg of silicified microcrystalline cellulose, about 0.05 mg to 3 mg of magnesium stearate, and about 0.05 mg to 3 mg of stearic acid; and a second matrix, wherein the second matrix comprises: about 5 mg to 30 mg of an antiemetic, about 100 mg to 250 mg of silicified microcrystalline cellulose, about 5 mg to 25 mg of croscarmellose sodium, and about 0.5 mg to 5 mg of magnesium stearate. In some instances, the first matrix comprises about 5 mg of the oxycodone or the pharmaceutically acceptable salt thereof, about 6.67 mg of croscarmellose sodium, about 5.6 mg of hydroxypropyl methylcellulose, about 60.24 mg of mannitol, about 120.49 mg of silicified microcrystalline cellulose, about 1 mg of magnesium stearate, and about 1 mg of stearic acid. In some instances, the second matrix comprises about 5 mg to 30 mg of the antiemetic, about 100 mg to 250 mg of silicified microcrystalline cellulose, about 5 mg to 25 mg of croscarmellose sodium, and about 0.5 mg to 5 mg of magnesium stearate. In some instances, the pharmaceutically acceptable salt of the oxycodone is oxycodone hydrochloride. In some instances, the oxycodone is present in about 2.5, about 5, about 7.5, about 10, about 15, or about 20 mg. In some instances, the pharmaceutically acceptable salt of the promethazine is promethazine hydrochloride. In some instances, the promethazine is present in about 12.5 or 25 mg. In some instances, the first matrix further comprises a non-opioid analgesic. In some instances, the non-opioid analgesic is acetaminophen. Provided herein are methods of treating or preventing pain, comprising orally administering to the subject any one of the pharmaceutical compositions described herein, wherein the pharmaceutical composition provides an effective amount of the opioid analgesic to treat or prevent pain in a subject in need thereof and an effective amount of the antiemetic to reduce or prevent an adverse effect associated with the opioid analgesic in the subject in need thereof. In some instances, the adverse effect associated with the opioid analgesic is nausea, vomiting, constipation, itching, gastric upset, or a skin rash. In some instances, the adverse effect is nausea or vomiting. In some instances, the adverse effect comprises nausea and vomiting. In some instances, the subject has a reduction in incidence of nausea or vomiting. In some instances, the subject has a reduction in intensity of nausea or vomiting. In some instances, the pharmaceutical composition is administered at least two times a day. In some instances, the pharmaceutical composition is administered at least three times a day. In some instances, the pharmaceutical composition is administered in a dosing interval of about 4 to about 6 hours. In some instances, the subject is a child. In some instances, the subject is an adult. In some instances, the subject is younger than 12 years of age. In sonic instances, the subject is 6 years of age or older. In some instances, the subject is 12 years of age or older.

### Active Agents

Provided herein are pharmaceutical compositions comprising a combination of an effective amount of an opioid analgesic with an effective amount of at least one other active ingredient. In some instances, the pharmaceutical composition is a solid oral pharmaceutical composition. Non-limiting examples of opioid analgesics include hydrocodone, oxycodone, acetyldihydrocodeinone, diamorphine, codeine, pethidine, alfentanil, buprenorphine, butorphanol, codeine, dezocine, fentanyl, hydromorphone, levomethadyl acetate, levorphanol, meperidine, methadone, morphine sulfate, nalbuphine, oxymorphone, pentazocine, propoxyphene, remifentanil, sufentanil, tramadol, or a pharmaceutically acceptable salt thereof. In some instances, the opioid analgesic agent is oxycodone or a pharmaceutically acceptable salt thereof. In some instances, the opioid analgesic agent is oxycodone sulphate, oxycodone hydrochloride, oxycodone trifluoroacetate, oxycodone thiosemicarbazone hydrochloride, oxycodone pentafluoropropionate, p-nitrophenylhydrazone oxycodone, o-methyloxine oxycodone, thiosemicarbazone oxycodone, semicarbazone oxycodone, phenylhydrazone oxycodone, hydrazone oxycodone, oxycodone hydrobromide, oxycodone mucate, oxycodone methylbromide, oxycodone oleate, n-oxide oxycodone, oxycodone acetate, dibasic oxycodone phosphate, oxycodone, monobasic oxycodone phosphate, inorganic or organic salts of oxycodone, oxycodone acetatetrihydrate, oxycodone bis (heptafluorobutyrate), oxycodone bis(methylcarbamate), oxycodone bis (pentafluoropropionate), oxycodone bis (pyridine-3-carboxylate), oxycodone his (trifluoroacetate), oxycodone bitartrate, oxycodone chlorohydrate, oxycodone pentahydrate sulfate, a pharmaceutically acceptable salt of any one of the foregoing, and any combination thereof. In some instances, the oxycodone or a pharmaceutically acceptable salt thereof is oxycodone hydrochloride. In some instances, the hydrocodone or a pharmaceutically acceptable salt thereof is hydrocodone bitartrate.

Provided herein are pharmaceutical compositions comprising a combination of an effective amount of an opioid analgesic with an effective amount of at least one other active ingredient. In some instances, the pharmaceutical composition is a solid oral pharmaceutical composition. In some instances, one of the at least one other active agent in combination with an opioid analgesic is an antiemetic. In some instances, a composition described herein comprises one or more antiemetics. In some instances, a composition described herein comprises 1, 2, 3, or 4 antiemetics. In some instances, a composition described herein comprises promethazine or a pharmaceutically acceptable salt thereof and a second antiemetic. In some instances, a composition described herein comprises ondansetron or a pharmaceutically acceptable salt thereof and a second antiemetic. Non-limiting examples of an antiemetic include aprepitant, dronabinol, perphenazine, palonosetron, trimethyobenzamide, metoclopramide, domperidone, prochlorperazine, promethazine, chlorpromazine, trimethobenzamide, ondansetron, granisetron, hydroxyzine, acetylleucine monoethanol amine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, thiethylperazine, thioproperazine, tropisetron, droperidol, haloperidol, prochloperazine, metoclopramide, diphenhydramine, midazolam, lorazepam, hyoscine, dexamethasone, emetrol, propofol, a pharmaceutically acceptable salt of any one of the foregoing, and any combination thereof. In some instances, the antiemetic is promethazine or a pharmaceutically acceptable salt thereof. In some instances, the promethazine or a pharmaceutically acceptable salt thereof is promethazine hydrochloride. In some instances, the antiemetic is a cannabinoid. Alternatively, a pharmaceutical composition described herein may comprise a cannabinoid to prevent or reduce pain.

Provided herein are pharmaceutical compositions comprising a combination of an effective amount of an opioid analgesic with an effective amount of at least one other active ingredient. In some instances, the pharmaceutical composition is a solid oral pharmaceutical composition. In some instances, one of the at least one other active agent in combination with the opioid analgesic is a non-opioid analgesic. Non-limiting examples of non-opioid analgesics include acetaminophen, acetylsalicylic acid, amoxiprin, benorilate, choline magnesium salicylate, diflunisal, faislamine, methyl salicylate, magnesium salicylate, diclofenac, aceclofenac, acemetacin, bromfenac, etodolac, indometacin, nabumetone, sulindac, tolmetin, ibuprofen, carprofen, fenbuprofen, flubiprofen, ketaprofen, ketorolac, loxoprofen, naproxen, suprofen, mefenamic acid, meclofenamic acid, piroxicam, lomoxicam, meloxicam, tenoxicam, phenylbutazone, azapropazone, metamizole, oxyphenbutazone, sulfinprazone, a pharmaceutically acceptable salt of any one of the foregoing, and any combination thereof. In some instances, the non-opioid analgesic is acetaminophen or a pharmaceutically acceptable salt thereof.

In some cases, each active agent described herein is used in the form of a free base, a pharmaceutically acceptable salt, a prodrug, an analog and/or a complex. As used herein, a pharmaceutically acceptable salt includes, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. In addition, pharmaceutically acceptable salts include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafluorobutyrate), bis(pentafluoropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically acceptable salt.

In some cases, a pharmaceutical composition as described herein comprises an effective amount of an opioid analgesic and an antiemetic. In some instances, the pharmaceutical composition is a solid oral pharmaceutical composition. In some instances, the opioid analgesic is oxycodone or a pharmaceutically acceptable salt thereof in some instances, the antiemetic is promethazine or a pharmaceutically acceptable salt thereof. In some instances, the opioid analgesic is oxycodone or a pharmaceutically acceptable salt thereof and the antiemetic is promethazine or a pharmaceutically acceptable salt thereof. In some instances, the pharmaceutically acceptable salt of oxycodone is oxycodone hydrochloride. In some instances, the pharmaceutically acceptable salt of promethazine is promethazine hydrochloride. In some instances, the opioid analgesic is hydrocodone or a pharmaceutically acceptable salt thereof. In some instances, the antiemetic is promethazine or a pharmaceutically acceptable salt thereof. In some instances, the opioid analgesic is hydrocodone or a pharmaceutically acceptable salt thereof and the antiemetic is promethazine or a pharmaceutically acceptable salt thereof. In some instances, the pharmaceutically acceptable salt of hydrocodone is hydrocodone bitartrate. In some instances, the pharmaceutically acceptable salt of promethazine is promethazine hydrochloride.

In some cases, a pharmaceutical composition as described herein comprises an effective amount of an opioid analgesic, a non-opioid analgesic, and an antiemetic. In some instances, the pharmaceutical composition is a solid oral pharmaceutical composition. In some instances, the opioid analgesic is hydrocodone or a pharmaceutically acceptable salt thereof. In some instances, the non-opioid analgesic is acetaminophen or a pharmaceutically acceptable salt thereof. In some instances, the antiemetic is promethazine or a pharmaceutically acceptable salt thereof. In some instances, the opioid analgesic is hydrocodone or a pharmaceutically acceptable salt thereof the non-opioid analgesic is acetaminophen or a pharmaceutically acceptable salt thereof, and the antiemetic is promethazine or a pharmaceutically acceptable salt thereof. In some instances, the pharmaceutically acceptable salt of hydrocodone is hydrocodone bitartrate. In some instances, the pharmaceutically acceptable salt of promethazine is promethazine hydrochloride.

Provided herein are pharmaceutical compositions comprising an effective amount of an opioid analgesic for the treatment or prevention of pain and an active agent useful for reducing or eliminating adverse effects associated with the opioid analgesic. In some instances, the pharmaceutical composition is a solid oral pharmaceutical composition. In some instances, pharmaceutical compositions described herein comprise an opioid analgesic and an antiemetic, wherein the antiemetic is the active agent useful for reducing or eliminating adverse effects associated with the opioid analgesic. In some instances, pharmaceutical compositions described herein comprise an opioid analgesic, a non-opioid analgesic, and an antiemetic.

As those of skill in the art would recognize, dosages and concentrations of active agents useful in the compositions described herein may be varied to achieve the effect desired. For example, the dosages administered can be adjusted based on the mode of administration, on the timing of administration and may be formulated to be administer once a day or for multiple administrations daily. In addition, dose levels can vary depending on the subject and/or condition being treated, the administration route used, as a function of the specific compound, as a function of the severity of the symptoms and/or the susceptibility of the subject to adverse effects.

In some cases, each active agent in the pharmaceutical composition is administered in a dosage of about 0.01 mg to about 500 mg per kg body weight per day, e.g., about 20 mg/day for an average person. In some instances, dosage for each active agent in the composition is from about 0.01 mg to about 5 mg, about 1 to about 10 mg, about 5 mg to about 20 mg, about 10 mg to about 50 mg, about 20 mg to about 100 mg, about 50 mg to about 150 mg, about 100 mg to about 250 mg, about 150 mg to about 300 mg, about 250 mg to about 500 mg, about 300 mg to about 600 mg or about 500 mg to about 1000 mg. In some instances, the pharmaceutical composition is a solid oral pharmaceutical composition.

Provided herein are solid oral pharmaceutical compositions, comprising: an outer matrix, wherein the outer matrix comprises polyalkylene oxide and one or more pharmaceutically acceptable excipients, and wherein the polyalkylene oxide has a molecular weight of at least about 7,000,000 g/mole; and an inner matrix, wherein the inner matrix comprises one or more active pharmaceutical ingredients. In some instances, the polyalkylene oxide is polyethylene oxide. In some instances, the composition further comprises an opioid analgesic. In some instances, the composition further comprises an anti-emetic. In some instances, the composition further comprises an opioid analgesic and an anti-emetic. In some instances, the composition further comprises an opioid, a non-opioid analgesic, and an anti-emetic. In some instances, the polyalkylene oxide is present in an amount of about 20 mg to about 50 mg. In some instances, the one or more pharmaceutically acceptable excipients includes a cellulose derivative. In some instances, the cellulose derivative is hydroxypropyl cellulose. In some instances, the cellulose derivative is present in an amount of about 1 mg to about 20 mg. In some instances, the one or more pharmaceutically acceptable excipients includes talc. In some instances, the talc is present in an amount of about 0.5 mg to about 10 mg. In some instances, the one or more pharmaceutically acceptable excipients includes a plasticizer. In some instances, the plasticizer is present in an amount of about 0.05 mg to about 5 mg. In some instances, the plasticizer is triethyl citrate. In some instances, the plasticizer is polyethylene glycol 3350. In some instances, the plasticizer is polyethylene glycol 400. In some instances, the plasticizer is selected from the group consisting of triacetin, polyethylene glycol 4000, and polyethylene glycol 6000. In some instances, the outer matrix comprises: about 20 mg to 50 mg of polyethylene oxide with a molecular weight of at least about 7,000,000 g/mole, about 1 mg to 20 mg of hydroxypropyl cellulose, about 0.5 mg to 10 mg of talc, and about 0.05 mg to 5 mg of triethyl citrate. In some instances, the outer matrix comprises: about 35 mg of polyethylene oxide with a molecular weight of at least about 7,000,000 g/mole, about 8.82 mg of hydroxypropyl cellulose, about 4.41 mg of talc, and about 1.77 mg of triethyl citrate. Provided herein are methods of treating or preventing pain, comprising orally administering to the subject any one of the pharmaceutical compositions described herein, wherein the pharmaceutical composition provides an effective amount of the opioid analgesic to treat or prevent pain in a subject in need thereof and an effective amount of the antiemetic to reduce or prevent an adverse effect associated with the opioid analgesic in the subject in need thereof. In some instances, the adverse effect associated with the opioid analgesic is nausea, vomiting, constipation, itching, gastric upset, or a skin rash. In some instances, the adverse effect is nausea or vomiting. In some instances, the adverse effect comprises nausea and vomiting. In some instances, the subject has a reduction in incidence of nausea or vomiting. In some instances, the subject has a reduction in intensity of nausea or vomiting. In some instances, the pharmaceutical composition is administered at least two times a day. In some instances, the pharmaceutical composition is administered at least three times a day. In some instances, the pharmaceutical composition is administered in a dosing interval of about 4 to about 6 hours. In some instances, the subject is a child. In some instances, the subject is an adult. In some instances, the subject is younger than 12 years of age. In some instances, the subject is 6 years of age or older. In some instances, the subject is 12 years of age or older.

Provided herein are solid oral pharmaceutical compositions, comprising: an outer matrix, wherein the outer matrix comprises a gelling agent and one or more pharmaceutically acceptable excipients, and wherein the gelling agent is about 40% to about 90% by weight of the total weight of the outer matrix; and one or more inner matrices, wherein the one or more inner matrices comprise an opioid analgesic or an antiemetic. In some instances, the gelling agent comprises a polyalkylene oxide. In some instances, the polyalkylene oxide is polyethylene oxide. In some instances, the gelling agent has a molecular weight of at least about 7,000,000 g/mol. In some instances, the gelling agent is present in an amount of about 20 mg to about 50 mg. In some instances, the gelling agent is about 10% to about 40% by weight of the total weight of the outer matrix. In some instances, the one or more pharmaceutically acceptable excipients includes a cellulose derivative. In some instances, cellulose derivative is hydroxypropyl cellulose. In some instances, the cellulose derivative is about 10% to about 37.5% by weight of the total weight of the outer matrix. In some instances, the cellulose derivative is present in an amount of about 1 mg to about 20 mg. In some instances, one or more pharmaceutically acceptable excipients includes talc. In some instances, the talc is about 1% to about 18.8% by weight of the total weight of the outer matrix. In some instances, the talc is present in an amount of about 0.5 mg to about 10 mg. In some instances, the one or more pharmaceutically acceptable excipients includes a plasticizer. In some instances, the plasticizer is about 1% to about 7.1% by weight of the total weight of the outer matrix. In some instances, the plasticizer is present in an amount of about 0.05 mg to about 5 mg. In some instances, the plasticizer is triethyl citrate. In some instances, the plasticizer is polyethylene glycol 3350. In some instances, the plasticizer is polyethylene glycol 400. In some instances, the plasticizer is selected from the group consisting of triacetin, polyethylene glycol 4000, and polyethylene glycol 6000. In some instances, the outer matrix comprises: about 20 mg to 50 mg of polyethylene oxide with a molecular weight of at least about 7,000,000 g/mole, about 1 mg to 20 mg of hydroxypropyl cellulose, about 0.5 mg to 10 mg of talc, and about 0.05 mg to 5 mg of triethyl citrate, in some instances, the outer matrix comprises: about 35 mg of polyethylene oxide with a molecular weight of at least about 7,000,000 Oriole, about 8.82 mg of hydroxypropyl cellulose, about 441 mg of talc, and about 1.77 mg of triethyl citrate. In some instances, at least one of the one or more inner matrices comprises an opioid, and wherein the opioid is oxycodone or a pharmaceutically acceptable salt thereof. In some instances, the pharmaceutically acceptable salt is oxycodone hydrochloride. In some instances, the oxycodone or pharmaceutically acceptable salt thereof is present in an amount of about 0.5 mg to about 20 mg. In some instances, the oxycodone or pharmaceutically acceptable salt thereof is present in an amount of about 2.5 mg, 5 mg, 110 mg, 15 mg. 20 mg, 25 mg, or 30 mg. In some instances, the oxycodone or pharmaceutically acceptable salt thereof is present in an amount of about 5 mg. In some instances, at least one of the one or more inner matrices comprises an opioid, and wherein the opioid is hydrocodone or a pharmaceutically acceptable salt thereof. In some instances, the pharmaceutically acceptable salt is hydrocodone bitartrate. In some instances, the hydrocodone or pharmaceutically acceptable salt thereof is present in an amount of about 2.5 mg to about 50 mg. In some instances, the hydrocodone or pharmaceutically acceptable salt thereof is present in an amount of about 7.5 mg. In some instances, the hydrocodone or pharmaceutically acceptable salt thereof is present in an amount of about 2.5 mg, 5 mg, 7.5 mg, 10 mg, 12.5 mg, 15 mg, 20 mg, 25 mg, 30 mg, 35 mg, 40 mg, 45 mg, or 50 mg. In some instances, at least one of the one or more inner matrices comprises an anti-emetic, and the anti-emetic is promethazine or a pharmaceutically acceptable salt thereof. In some instances, the pharmaceutically acceptable salt is promethazine hydrochloride. In some instances, promethazine or a pharmaceutically acceptable salt thereof is present in an amount of about 5 mg to about 30 mg. In some instances, promethazine or a pharmaceutically acceptable salt thereof is present in an amount of about 12.5 mg. In some instances, promethazine or a pharmaceutically acceptable salt thereof is present in an amount of about 12.5 mg, 15 mg, 20 mg, 25 mg, 30 mg, 35 mg, 40 mg, 45 mg, or 50 mg. In some instances, at least one of the one or more inner matrices further comprises a non-opioid analgesic. In some instances, the non-opioid analgesic is acetaminophen. In some instances, the acetaminophen is present in an amount of about 200 mg to about 600 mg. In some instances, the acetaminophen is present in an amount of about 360 mg. In some instances, the acetaminophen is present in an amount of about 325 mg. In some instances, at least one of the one or more inner matrices further comprises a first one or more pharmaceutically acceptable excipients. Provided herein are methods of treating or preventing pain, comprising orally administering to the subject any one of the pharmaceutical compositions described herein, wherein the pharmaceutical composition provides an effective amount of the opioid analgesic to treat or prevent pain in a subject in need thereof and an effective amount of the antiemetic to reduce or prevent an adverse effect associated with the opioid analgesic in the subject in need thereof. In some instances, the adverse effect associated with the opioid analgesic is nausea, vomiting, constipation, itching, gastric upset, or a skin rash. In some instances, the adverse effect is nausea or vomiting. In some instances, the adverse effect comprises nausea and vomiting. In some instances, the subject has a reduction in incidence of nausea or vomiting. In some instances, the subject has a reduction in intensity of nausea or vomiting. In some instances, the pharmaceutical composition is administered at least two times a day. In some instances, the pharmaceutical composition is administered at least three times a day. In some instances, the pharmaceutical composition is administered in a dosing interval of about 4 to about 6 hours. In some instances, the subject is a child. In some instances, the subject is an adult. In some instances, the subject is younger than 12 years of age. In some instances, the subject is 6 years of age or older. In some instances, the subject is 12 years of age or older.

Provided herein are solid oral pharmaceutical compositions, comprising: an outer matrix, wherein the outer matrix comprises a gelling agent; and two or more inner matrices, wherein a first inner matrix comprises oxycodone and a second inner matrix comprises promethazine. In some instances, the gelling agent is polyalkylene oxide. In some instances, the gelling agent is polyethylene oxide. In some instances, the gelling agent has a molecular weight of at least about 7,000,000 g/mol. In some instances, the gelling agent is present in an amount of about 20 mg to about 50 mg. In some instances, the first inner matrix is a controlled-release layer. In some instances, the second inner matrix is an immediate-release layer. In some instances, the second inner matrix has a faster dissolution rate than the first inner matrix. In some instances, the promethazine is released before the oxycodone. In some instances, at least about 62% of the oxycodone is released within the first 5 minutes. In some instances, at least about 78% of the oxycodone is released within the first 5 minutes. In some instances, at least about 69% of the oxycodone is released within the first 10 minutes. In some instances, at least about 72% of the oxycodone is released within the first 10 minutes. In some instances, at least about 84% of the oxycodone is released within the first 10 minutes. In some instances, at least about 73% of the oxycodone is released within the first 15 minutes. In some instances, at least about 76% of the oxycodone is released within the first 15 minutes. In some instances, at least about 87% of the oxycodone is released within the first 15 minutes. In some instances, at least about 78% of the oxycodone is released within the first 30 minutes. In some instances, at least about 83% of the oxycodone is released within the first 30 minutes. In sonic instances, at least about 92% of the oxycodone is released within the first 30 minutes. In some instances, at least about 90% of the oxycodone is released within the first 60 minutes. In some instances, at least about 97% of the oxycodone is released within the first 60 minutes. In some instances, at least about 90% of the promethazine is released within the first 5 minutes. In some instances, at least about 81% of the promethazine is released within the first 5 minutes. In some instances, at least about 77% of the promethazine is released within the first 5 minutes. In some instances, at least about 94% of the promethazine is released within the first 10 minutes. In some instances, at least about 85% of the promethazine is released within the first 10 minutes. In some instances, at least about 78% of the promethazine is released within the first 10 minutes. In some instances, at least about 96% of the promethazine is released within the first 15 minutes. In some instances, at least about 86% of the promethazine is released within the first 15 minutes. In some instances, at least about 81% of the promethazine is released within the first 15 minutes. In some instances, at least about 97% of the promethazine is released within the first 30 minutes. In some instances, at least about 88% of the promethazine is released within the first 30 minutes. In some instances, at least about 87% of the promethazine is released within the first 30 minutes. In some instances, at least about 97% of the promethazine is released within the first 60 minutes. In some instances, at least about 88% of the promethazine is released within the first 60 minutes. In some instances, least about 87% of the promethazine is released within the first 60 minutes. In some instances, the pharmaceutically acceptable salt of oxycodone is oxycodone hydrochloride. In some instances, the oxycodone is present in an amount of about 0.5 mg to about 20 mg. In some instances, the pharmaceutically acceptable salt of promethazine is promethazine hydrochloride. In some instances, the promethazine is present in an amount of about 5 mg to about 30 mg. In some instances, the first inner matrix comprises: about 0.5 mg to 20 mg of oxycodone, about 1 mg to 15 mg of croscarmellose sodium, about 1 mg to 20 mg of hydroxypropyl methylcellulose, about 10 mg to 100 mg of lactose monohydrate, about 50 mg to 200 mg of silicified microcrystalline cellulose, about 0.05 mg to 3 mg of magnesium stearate, and about 0.05 mg to 3 mg of stearic acid. In some instances, the first matrix comprises: about 5 mg of oxycodone hydrochloride, about 6.67 mg of croscarmellose sodium, about 10.33 mg of hydroxypropyl methylcellulose, about 58.67 mg of lactose monohydrate, about 117.33 mg of silicified microcrystalline cellulose, about 1 mg of magnesium stearate, and about 1 mg of stearic acid. In some instances, the first inner matrix comprises about 0.5 mg to 20 mg of oxycodone, about 1 mg to 15 mg of croscarmellose sodium, about 1 mg to 20 mg of hydroxypropyl methylcellulose, about 10 mg to 100 mg of mannitol, about 50 mg to 200 mg of silicified microcrystalline cellulose, about 0.05 mg to 3 mg of magnesium stearate, and about 0.05 mg to 3 mg of stearic acid. In some instances, the first matrix comprises: about 5 mg of oxycodone hydrochloride, about 6.67 mg of croscarmellose sodium, about 5.6 mg of hydroxypropyl methylcellulose, about 60.24 mg of mannitol, about 120.49 mg of silicified microcrystalline cellulose, about 1 mg of magnesium stearate, and about 1 mg of stearic acid. In some instances, the first inner matrix comprises: about 0.5 mg to 2.0 mg of oxycodone, about 0.2 mg to 5 mg of hydroxypropyl methylcellulose, about 15 mg to 50 mg of silicified microcrystalline cellulose, about 0.05 mg to 3 mg of magnesium stearate, about 0.05 mg to 3 mg of stearic acid, and about 0.2 mg to 5 mg of sodium starch glycolate. In some instances, the second inner matrix comprises: about 5 mg to 30 mg of promethazine, about 75 mg to 150 mg of silicified microcrystalline cellulose, about 5 mg to 30 mg of croscarmellose sodium, and about 0.05 mg to about 3 mg of magnesium stearate. In some instances, the second inner matrix comprises: about 12.5 mg of promethazine hydrochloride, about 121.5 mg of silicified microcrystalline cellulose, about 15 mg of croscarmellose sodium, and about 1 mg of magnesium stearate. In some instances, the outer matrix further comprises: about 1 mg to 20 mg of hydroxypropyl cellulose, about 0.5 mg to 10 mg of talc, and about 0.05 mg to 5 mg of triethyl citrate. In some instances, the outer matrix comprises: about 35 mg of polyethylene oxide with a molecular weight of about 7,000,000 g/mole, about 8.82 mg of hydroxypropyl cellulose, about 4.41 mg of talc, and about 1.77 mg of triethyl citrate. Provided herein are methods of treating or preventing pain, comprising orally administering to the subject any one of the pharmaceutical compositions described herein, wherein the pharmaceutical composition provides an effective amount of the opioid analgesic to treat or prevent pain in a subject in need thereof and an effective amount of the anti emetic to reduce or prevent an adverse effect associated with the opioid analgesic in the subject in need thereof. In some instances, the adverse effect associated with the opioid analgesic is nausea, vomiting, constipation, itching, gastric upset, or a skin rash. In some instances, the adverse effect is nausea or vomiting. In some instances, the adverse effect comprises nausea and vomiting. In some instances, the subject has a reduction in incidence of nausea or vomiting. In some instances, the subject has a reduction in intensity of nausea or vomiting. In some instances, the pharmaceutical composition is administered at least two times a day. In some instances, the pharmaceutical composition is administered at least three times a day. In some instances, the pharmaceutical composition is administered in a dosing interval of about 4 to about 6 hours. In some instances, the subject is a child. In some instances, the subject is an adult. In some instances, the subject is younger than 12 years of age. In some instances, the subject is 6 years of age or older. In some instances, the subject is 12 years of age or older.

Provided herein are solid oral pharmaceutical compositions, comprising: an outer matrix, wherein the outer matrix comprises a gelling agent; and two or more inner matrices, wherein a first inner matrix comprises hydrocodone and a second inner matrix comprises promethazine. In some instances, the gelling agent is polyalkylene oxide. In some instances, the gelling agent is polyethylene oxide. In some instances, the gelling agent has a molecular weight of at least about 7,000,000 g/mol. In some instances, the gelling agent is present in an amount of about 20 mg to about 50 mg. In some instances, the first inner matrix is a controlled-release layer. In some instances, the second inner matrix is an immediate-release layer. In some instances, the second inner matrix has a faster dissolution rate than the first inner matrix. In some instances, the promethazine is released before the hydrocodone. In some instances, the pharmaceutically acceptable salt of hydrocodone is hydrocodone bitartrate. In some instances, the hydrocodone is present in an amount of about 0.5 mg to about 50 mg. In some instances, the pharmaceutically acceptable salt of promethazine is promethazine hydrochloride. In some instances, the promethazine is present in an amount of about 5 mg to about 30 mg. In some instances, the composition further comprises a non-opioid analgesic. In some instances, the non-opioid analgesic is present in an amount of about 200 mg to about 600 mg. In some instances, the non-opioid analgesic is acetaminophen. In some instances, the first inner matrix comprises: about 2.5 mg to 50 mg of hydrocodone, about 5 mg to 2.0 mg of croscarmellose sodium, about 100 mg to 250 mg of microcrystalline cellulose, about 5 mg to 25 mg of hydroxypropyl methylcellulose about 0.5 mg to 5 mg of magnesium stearate, and about 0.5 mg to 5 mg of stearic acid. In some instances, the first inner matrix further comprises: about 200 mg to 600 mg of acetaminophen. In some instances, the first inner matrix comprises: about 7.5 mg of hydrocodone bitartrate, about 10 mg of croscarmellose sodium, about 150.4 mg of microcrystalline cellulose, about 15.5 mg, of hydroxypropyl methylcellulose about 2.75 mg of magnesium stearate, and about 2.75 mg to 5 mg of stearic acid. In some instances, the first inner matrix further comprises: about 325 mg acetaminophen. In some instances, the second inner matrix comprises: about 5 mg to 30 mg of promethazine, about 100 mg to 250 mg of microcrystalline cellulose, about 5 mg to 25 mg of croscarmellose sodium, and about 0.5 mg to 5 mg of magnesium stearate. In some instances, the second inner matrix comprises: about 12.5 mg of promethazine, about 121.5 mg of microcrystalline cellulose, about 15 mg of croscarmellose sodium, and about 1 mg of magnesium stearate. In some instances, the outer matrix further comprises: about 1 mg to 20 mg of hydroxypropyl cellulose, about 0.5 mg to 10 mg of talc, and about 0.05 mg to 5 mg of triethyl citrate. In some instances, the outer matrix comprises: about 35 mg of polyethylene oxide with a molecular weight of at least about 7,000,000 g/mole, about 8.82 mg of hydroxypropyl cellulose, about 4.41 mg of talc, and about 1.77 mg of triethyl citrate. Provided herein are methods of treating or preventing pain, comprising orally administering to the subject any one of the pharmaceutical compositions described herein, wherein the pharmaceutical composition provides an effective amount of the opioid analgesic to treat or prevent pain in a subject in need thereof and an effective amount of the antiemetic to reduce or prevent an adverse effect associated with the opioid analgesic in the subject in need thereof. In some instances, the adverse effect associated with the opioid analgesic is nausea, vomiting, constipation, itching, gastric upset, or a skin rash. In some instances, the adverse effect is nausea or vomiting. In some instances, the adverse effect comprises nausea and vomiting. In some instances, the subject has a reduction in incidence of nausea or vomiting. In some instances, the subject has a reduction in intensity of nausea or vomiting. In some instances, the pharmaceutical composition is administered at least two times a day. In some instances, the pharmaceutical composition is administered at least three times a day. In some instances, the pharmaceutical composition is administered in a dosing interval of about 4 to about 6 hours. In some instances, the subject is a child. In some instances, the subject is an adult. In some instances, the subject is younger than 12 years of age. In some instances, the subject is 6 years of age or older. In some instances, the subject is 12 years of age or older.

Provided herein are solid oral pharmaceutical compositions comprising: a first matrix comprising an opioid analgesic, a second matrix comprising an anti-emetic, and a third matrix comprising a gelling agent, wherein the gelling agent has a molecular weight of at least about 7,000,000 g/mol. In some instances, the third matrix surrounds the first matrix. In some instances, the third matrix surrounds the first matrix and the second matrix. In some instances, the first matrix is a controlled-release layer. In some instances, the second matrix is an immediate-release layer. In some instances, the second matrix has a faster dissolution rate than the first matrix. In some instances, the anti-emetic is released before the opioid analgesic. In some instances, the gelling agent is polyalkylene oxide. In some instances, the gelling agent is polyethylene oxide. In some instances, the gelling agent has a molecular weight of 7,000,000 g/mol. In some instances, the gelling agent is present in an amount of about 20 mg to about 50 mg. In some instances, the anti-emetic is promethazine or a pharmaceutically acceptable salt thereof. In some instances, the anti-emetic is present in an amount of about 5 mg to about 30 mg. In some instances, the anti-emetic is promethazine hydrochloride. In some instances, the promethazine hydrochloride is present in an amount of about 12.5 mg. In some instances, the opioid analgesic is oxycodone or a pharmaceutically acceptable salt thereof. In some instances, the opioid analgesic is present in an amount of about 0.5 mg to about 20 mg. In some instances, the opioid analgesic is oxycodone hydrochloride. In some instances, the oxycodone hydrochloride is present in an amount of about 5 mg. In some instances, the opioid analgesic is present in an amount of about 0.5 mg to about 50 mg. In some instances, the opioid analgesic is hydrocodone or a pharmaceutically acceptable salt thereof. In some instances, the opioid analgesic is hydrocodone bitartrate. In some instances, the hydrocodone bitartrate is present in an amount of about 7.5 mg. In some instances, the second matrix further comprises a non-opioid analgesic. In some instances, the non-opioid analgesic is present in an amount of about 200 mg to about 600 mg. In some instances, the non-opioid analgesic is acetaminophen or a pharmaceutically acceptable salt thereof. In some instances, the acetaminophen is present in an amount of about 325 mg. Provided herein are methods of treating or preventing pain, comprising orally administering to the subject any one of the pharmaceutical compositions described herein, wherein the pharmaceutical composition provides an effective amount of the opioid analgesic to treat or prevent pain in a subject in need thereof and an effective amount of the antiemetic to reduce or prevent an adverse effect associated with the opioid analgesic in the subject in need thereof. In some instances, the adverse effect associated with the opioid analgesic is nausea, vomiting, constipation, itching, gastric upset, or a skin rash. In some instances, the adverse effect is nausea or vomiting. In some instances, the adverse effect comprises nausea and vomiting. In some instances, the subject has a reduction in incidence of nausea or vomiting. In some instances, the subject has a reduction in intensity of nausea or vomiting. In some instances, the pharmaceutical composition is administered at least two times a day. In some instances, the pharmaceutical composition is administered at least three times a day. In some instances, the pharmaceutical composition is administered in a dosing interval of about 4 to about 6 hours. In some instances, the subject is a child. In some instances, the subject is an adult. In some instances, the subject is younger than 12 years of age. In some instances, the subject is 6 years of age or older. In some instances, the subject is 12 years of age or older.

### Opioid Analgesic

In some cases, pharmaceutical compositions described herein comprise an opioid analgesic, wherein the opioid analgesic has a purity of 60-100% by weight. In some instances, the opioid analgesic has a purity of 70-100%, 80-100%, 90-100%, 95-100%, or 85-95% by weight. In some instances, the opioid analgesic has a purity of at least 70%, 75%, 80%, 85%, 90%, 95%, 96%, 97%, 98%, 99%, or 100% by weight. In some instances, the pharmaceutical composition is a solid oral pharmaceutical composition.

In some cases, an opioid analgesic is present in an effective amount. In some instances, an opioid analgesic is present in an amount that is effective for treating, reducing, or preventing pain. In some instances, an opioid analgesic is present in an amount of about 0.5 mg to about 100 mg, including any number within this range. In some instances, an opioid analgesic is present in an amount of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, about 50 mg, about 55 mg, about 60 mg, about 65 mg, about 70 mg, about 75 mg, about 80 mg, about 85 mg, about 90 mg, about 95 mg, or about 100 mg. In further instances, the disclosed amount of the opioid analgesic refers to a total weight amount of the opioid analgesic in a salt form or a therapeutically active portion thereof.

In some cases, an opioid analgesic is present in an amount that is effective for treating, reducing, or preventing pain. In some instances, the opioid analgesic is oxycodone or a pharmaceutically acceptable salt thereof. In some instances, the oxycodone or a pharmaceutically acceptable salt thereof is oxycodone hydrochloride. In some instances, the oxycodone hydrochloride is present in an amount of about 0.5 mg to about 20 mg. In some instances, the oxycodone hydrochloride is present in an amount of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, or about 20 mg. In some instances, the oxycodone hydrochloride is present in an amount of about 5 mg, about 7.5 mg, about 10 mg, about 15 mg, or about 20 mg. In some instances, the oxycodone hydrochloride is present in an amount of about 5 mg.

In some cases, an opioid analgesic is present in an amount that is effective for treating, reducing, or preventing pain. In some instances, the opioid analgesic is hydrocodone or a pharmaceutically acceptable salt thereof. In some instances, the hydrocodone or a pharmaceutically acceptable salt thereof is hydrocodone bitartrate. In some instances, the hydrocodone bitartrate is present in an amount of about 0.5 mg to about 20 mg. In some instances, the hydrocodone bitartrate is present in an amount of about 0.5 mg, about 1 mg, about 1.5 mg, about 2 mg, about 2.5 mg, about 3 mg, about 3.5 mg, about 4 mg, about 4.5 mg, about 5 trig, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, or about 20 mg. In some instances, the hydrocodone bitartrate is present in an amount of about 5 mg, about 7.5 mg, about 10 mg, about 15 mg, or about 20 mg. In some instances, the hydrocodone bitartrate is present in an amount of about 7.5 mg.

In some cases, pharmaceutical compositions described herein comprise between about 0.5% to about 15% by weight of an opioid analgesic. In some instances, the pharmaceutical composition comprises about 0.5%, 0.6%, 0.7%, 0.8%, 0.9%, 1%, 1.1%, 1.2%, 1.2%, 1.3%, 1.4%, 1.5%, 1.6%, 1.7%, 1.8%, 1.9%, 2%, 2.1%, 2.2%, 2.2%, 2.3% 2.4%, 2.5%, 2.6%, 2.7%, 2.8%, 2.9%, 3%, 3.1%, 3.2%, 3.2%, 3.3%, 3.4%, 3.5%, 3.6%, 3.7%, 3.8%, 3.9%, 4%, 4.1%, 4.2%, 4.2%, 4.3%, 4.4%, 4.5%, 4.6%, 4.7%, 4.8%, 4.9%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% by weight of an opioid analgesic. In some instances, the opioid analgesic is oxycodone or a pharmaceutically acceptable salt thereof. In some instances, the oxycodone or a pharmaceutically^{,} acceptable salt thereof is oxycodone hydrochloride. In some instances, the opioid analgesic is hydrocodone or a phannaceuticaliy acceptable salt thereof. In some instances, the hydrocodone or a pharmaceutically acceptable salt thereof is hydrocodone bitartrate. In some instances, the pharmaceutical composition is a solid oral pharmaceutical composition.

### Antiemetic

In some cases, pharmaceutical compositions described herein comprise an antiemetic, wherein the antiemetic has a purity of 60-100% by weight. In some instances, pharmaceutical compositions described herein comprise an antiemetic, wherein the antiemetic has a purity of 70-100%, 80-100%, 90-100%, 95-100%, or 85-95% by weight. In some instances, pharmaceutical compositions described herein comprise an antiemetic, wherein the antiemetic has a purity of at least 70%, 75%, 80%, 85%, 90%, 95%, 96%, 97%, 98%, 99%, or 100% by weight. In some instances, the pharmaceutical composition is a solid oral pharmaceutical composition. In some instances, an antiemetic is present in an effective amount. In some instances, an antiemetic is present in an amount that is effective for preventing, reducing or eliminating one or more adverse effects associated with the opioid analgesic in a subject in need thereof. In some instances, a combination of antiemetics are present in an amount that is effective for preventing, reducing or eliminating one or more adverse effects associated with the opioid analgesic in a subject in need thereof. In some instances, the antiemetic is present in an amount of from about 1 mg to about 100 mg, including in the amount of any single number within this range. In some instances, the antiemetic is present in an amount of about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 22.5 mg, about 2.5 mg, about 27.5 mg, about 30 mg, about 32.5 mg, about 35 mg, about 37.5 mg, about 40 mg, about 42.5 mg, about 45 mg, about 47.5 mg, about 50 mg, about 55 mg, about 60 mg, about 65 mg, about 70 mg, about 75 mg, about 80 mg, about 85 mg, about 90 mg, about 95 mg, or about 100 mg. In further instances, the disclosed amount of the anti emetic refers to a total weight amount of the antiemetic in a salt form or a therapeutically active portion thereof.

In some cases, a combination of antiemetics are present in an amount that is effective for preventing, reducing or eliminating one or more adverse effects associated with the opioid analgesic in a subject in need thereof, in some instances, the antiemetic is promethazine or a pharmaceutically acceptable salt thereof. In some instances, the promethazine or a pharmaceutically acceptable salt thereof is promethazine hydrochloride. In some instances, the promethazine hydrochloride is present in an amount of about 5 mg to about 30 mg. In some instances, the promethazine hydrochloride is present in an amount of about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, or about 30 mg, in some instances, the promethazine hydrochloride is present in an amount of about 12.5 mg or about 25 mg. In some instances, the promethazine hydrochloride is present in an amount of about 12.5 mg. In some instances, the promethazine or a pharmaceutically acceptable salt thereof is present in an amount of about 12.5 mg to 60 mg. In some instances, the antiemetic is ondansetron or a pharmaceutically acceptable salt thereof. In some instances, the ondansetron or a pharmaceutically acceptable salt thereof is ondansetron hydrochloride. In some instances, the ondansetron hydrochloride is present in an amount of about 5 mg to about 30 mg. In some instances, the ondansetron or a pharmaceutically acceptable salt thereof is present in an amount of about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 2.0 mg, about 25 mg, or about 30 mg.

In some cases, pharmaceutical compositions described herein comprises between about 0.1% to about 20% by weight of an antiemetic. In some instances, the pharmaceutical composition comprises about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 3%, 4%, 5%, 6%, 7%, 8%, 9%, 10%, 11%, 12%, 13%, 14%, 15%, 16%, 17%, 18%, 19%, or 20% by weight of an antiemetic. In some instances, the antiemetic is promethazine or a pharmaceutically acceptable salt thereof. In some instances, the promethazine or a pharmaceutically acceptable salt thereof is promethazine hydrochloride. In some instances, the antiemetic is ondansetron or a pharmaceutically acceptable salt thereof. In some instances, the ondansetron or a pharmaceutically acceptable salt thereof is ondansetron hydrochloride. In some instances, the pharmaceutical composition is a solid oral pharmaceutical composition.

In some cases, a pharmaceutical composition described herein comprises an effective amount of an opioid analgesic and an effective amount of an antiemetic. In some instances, the ratio by weight of the opioid analgesic to the antiemetic is between about 0.1:1 to about 1:1. In some instances, the ratio by weight of the opioid analgesic to the antiemetic is about 0.1:1, about 0.2:1, about 0.3:1, about 0.4:1, about 0.5:1, about 0.6:1, about 0.7:1, about 0.8:1, about 0.9:1, or about 1:1. In some instances, the ratio by weight of the opioid analgesic to the antiemetic is about 0.4:1. In some instances, the pharmaceutical composition is a solid oral pharmaceutical composition. In some instances, the pharmaceutical composition comprises an opioid analgesic in an amount of from about 0.5 mg to about 100 mg; and an antiemetic in an amount of from about 1 mg to about 100 mg. In some instances, the pharmaceutical composition comprises oxycodone or a pharmaceutically acceptable salt thereof in an amount of from about 0.5 mg to about 20 mg and promethazine or a pharmaceutically acceptable salt thereof in an amount of from about 5 mg to about 30 mg. In some instances, the compositions comprise about 5 mg of oxycodone or a pharmaceutically acceptable salt thereof and about 12.5 mg of promethazine or a pharmaceutically acceptable salt thereof. In some instances, the compositions comprise about 5 mg of oxycodone hydrochloride and about 12.5 mg of promethazine hydrochloride. In some instances, the pharmaceutical composition comprises hydrocodone or a pharmaceutically acceptable salt thereof in an amount of from about 0.5 mg to about 50 mg and promethazine or a pharmaceutically acceptable salt thereof in an amount of from about 5 mg to about 30 mg. In some instances, the compositions comprise about 7.5 mg of hydrocodone or a phartnaceutically acceptable salt thereof and about 12.5 mg of promethazine or a pharmaceutically acceptable salt thereof. In some instances, the compositions comprise about 7.5 mg of hydrocodone bitartrate and about 12.5 mg of promethazine hydrochloride. In some instances, the pharmaceutical composition is a solid oral pharmaceutical composition.

### Non-Opioid Analgesic

In some cases, a pharmaceutical compositions described herein comprise a non-opioid analgesic, wherein the non-opioid analgesic has a purity of 60-100% by weight. In some instances, the compositions described herein comprise a non-opioid analgesic, wherein the non-opioid analgesic has a purity of 70-100%, 80-100%, 90-100%, 95-100%, or 85-95% by weight. In some instances, the compositions described herein comprise a non-opioid analgesic, wherein the non-opioid analgesic has a purity of at least 70%, 75%, 80%, 85%, 90%, 95%, 96%, 97%, 98%, 99%, or 100% by weight. In some instances, the pharmaceutical composition is a solid oral pharmaceutical composition.

In some cases, a non-opioid analgesic is present in an effective amount. In some instances, a non-opioid analgesic is present in an amount of about 200 mg to about 600 mg, including any single number within this range. In some instances, a non-opioid analgesic is present in an amount of about 200 mg to about 600 mg, about 200 mg to about 1000 mg, about 200 mg to about 325 mg, about 325 mg to about 330 mg, about 330 mg to about 335 mg, about 335 mg to about 340 mg, about 340 mg to about 345 mg, about 345 mg to about 350 mg, about 325 mg to about 350 mg, about 350 mg to about 400 mg, about 400 mg to about 1000 mg, or any combination thereof. In some instances, a non-opioid analgesic is present in an amount of about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg. about 525 mg. about 550 mg, about 575 mg, about 600 mg, about 625 mg, or about 650 mg. In further instances, the disclosed amount of the non-opioid analgesic refers to a total weight amount of the non-opioid analgesic in a salt form or a therapeutically active portion thereof.

In some cases, a pharmaceutical composition comprises an opioid analgesic in an amount of from about 0.5 mg to about 100 mg; a non-opioid analgesic in an amount of from about 200 mg to about 600 mg; and an antiemetic in an amount of from about 1 mg to about 100 mg. In some instances, a pharmaceutical composition comprises hydrocodone or a pharmaceutically acceptable salt thereof in an amount of from about 0.5 mg to about 20 mg, acetaminophen or a pharmaceutically acceptable salt thereof in an amount of from about 200 mg to about 600 mg; and promethazine or a pharmaceutically acceptable salt thereof in an amount of from about 5 mg to about 30 mg. In some instances, a pharmaceutical composition comprises about 7.5 mg of hydrocodone or a pharmaceutically acceptable salt thereof, about 325 mg acetaminophen, and about 12.5 mg of promethazine or a pharmaceutically acceptable salt thereof. In some instances, a pharmaceutical composition comprises about 7.5 mg of hydrocodone bitartrate, about 325 mg acetaminophen, and about 12.5 mg of promethazine hydrochloride. In some instances, the pharmaceutical composition is a solid oral pharmaceutical composition.

In some cases, a pharmaceutical composition comprises between about 30% to about 70% by weight of a non-opioid analgesic. In some instances, a pharmaceutical composition. comprises 30%, 31%, 32%, 33%, 34%, 35%, 36%, 37%, 38%, 39%, 40%, 41%, 42%, 41%, 44%, 45%, 46%, 47%, 48%, 49%, 50%, 51%, 51.5%, 51.6%, 52%, 53%, 54%, 55%, 56%, 57%, 58%, 59%, 60%, 61%, 62%, 63%, 64%, 65%, 66%, 67%, 68%, 69%, or 70% by weight of a non-opioid analgesic. In some instances, a pharmaceutical composition comprises between about 30% to about 70% by weight of acetaminophen. In some instances, a pharmaceutical composition comprises 30%, 31%, 32%, 33%, 34%, 35%, 36%, 37%, 38%, 39%, 40%, 41%, 42%, 43%, 44%, 45%, 46%, 47%, 48%, 49%, 50%, 51%, 51.5%, 51.6%, 52%, 53%, 54%, 55%, 56%, 57%, 58%, 59%, 60%, 61%, 62%, 63%, 64%, 65%, 66%, 67%, 68%, 69% or 70% by weight of acetaminophen. In some instances, the pharmaceutical composition is a solid oral pharmaceutical composition.

### Cannabinoid

In some cases, a pharmaceutical composition disclosed herein comprises a cannabinoid for the treatment, reduction or prevention of pain. In some instances, the pharmaceutical composition is a solid oral pharmaceutical composition. Exemplary cannabinoid for the treatment of pain include, without limitation, nabilone, dronabinol (THC), cannabidiol (CBD), cannabinol (CBN), cannabichromeme (CBC), cannabigerol (CBG), tetrahydrocannabivarin (THCV), tetrahydrocannabinolic acid (THCA), cannabidivarin (CBDV), cannadidiolic acid (CBDA), ajulemic acid, dexanabinol, cannabinor, HU 308, HU 331, and a pharmaceutically acceptable salt thereof. In some instances, the cannabinoid is nabilone or a pharmaceutically acceptable salt thereof. In some instances, the cannabinoid is dronabinol (THC) or a pharmaceutically acceptable salt thereof. In some instances, the cannabinoid is provided at a dose to prevent or reduce pain. In some instances, the pharmaceutical composition described herein comprises a pharmaceutically acceptable salt of a cannabinoid in a quantity therapeutically equivalent to cannabinoid dosages disclosed herein. In some instances, the pharmaceutical composition disclosed herein comprises cannabinoid (e.g., nabilone, or a pharmaceutically acceptable salt thereof) present at a dose of from about 0.5 mg to about 50 mg, including, but not limited to, from about 0.5 mg to about 25 mg, from about 25 mg to about 50 mg, from about 0.5 mg to about 5 mg, from about 5 mg to about 10 mg, from about 10 mg to about 20 mg, from about 20 mg to about 30 mg, from about 30 mg to about 40 mg, or from about 40 Ing to about 50 mg. In some instances, the pharmaceutical composition disclosed herein comprises cannabinoid (e.g., nabilone, or a pharmaceutically acceptable salt thereof) present at a dose of about 1 mg. In some instances, the pharmaceutical composition is a solid oral pharmaceutical composition.

### Excipients/Carriers/Additives

In some cases, a pharmaceutical composition described herein comprises one or more excipients, carriers, or additives. In some instances, the pharmaceutical composition is a solid oral pharmaceutical composition. Examples of such excipients, carriers, or additives can be found in Remington's Pharmaceutical Sciences, 17th edition (1985). Suitable additives include, but are not limited to, gelling agents, diluents, disintegrants, binders, lubricants, glidants, plasticizers, coloring agents, or pharmaceutically inert materials. Examples of these additives are provided herein.

### Binders

In some cases, a pharmaceutical composition described herein comprises a binder. In some instances, the pharmaceutical composition is a solid oral pharmaceutical composition. Exemplary binders include celluloses such as hydroxypropylcellulose, methylcellulose, and hydroxypropylmethyl cellulose; starches such as corn starch, pregelatinized starch, and hydroxypropyl starch; sugars such as glucose, dextrose, sucrose, lactose, lactose monohydrate, and sorbitol; alcohols such as polyvinyl alcohol and polyethylene glycol; mannitol; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof. Binders impact cohesive qualities to a tablet formulation, or a particle formulation in a capsule. Tablets remain intact after compression by including a binder in the pharmaceutical composition.

### Plasticizers

In some cases, a pharmaceutical compositions described herein comprise a plasticizer. In some instances, the pharmaceutical composition is a solid oral pharmaceutical composition. Exemplary plasticizers include triethyl citrate, triacetin, polyethylene glycol 400, polyethylene glycol 3350, polyethylene glycol 4000, polyethylene glycol 6000, or combinations thereof.

### Coloring Agents

In some cases, a pharmaceutical composition described herein comprises a coloring agent. In some instances, the pharmaceutical composition is a solid oral pharmaceutical composition. Exemplary coloring agents include one or more synthetic organic food additives (e.g., food dyes such as food red dye Nos. 2 and 3, food yellow dye Nos. 4 and 5 and food blue dye Nos. 1 and 2), water-insoluble lake dyes (e.g., aluminum salts of the above synthetic organic food additives, etc.), natural pigments (e.g., beta-carotene, chlorophyll, iron oxide red, etc.), or combinations thereof. In some instances, other suitable colorant agents include D&C Red No. 33, FD&C Red No. 3, FD&C Red No. 40, D&C Yellow No. 10, and C Yellow No. 6, or any combination of these or the above colorants.

### Diluents

In some cases, a pharmaceutical composition described herein comprises a diluent. In some instances, the pharmaceutical composition is a solid oral pharmaceutical composition. Exemplary diluents include cellulose and cellulose derivatives such as microcrystalline cellulose; starches such as dry starch, hydrolyzed starch, and starch derivatives such as corn starch; cyclodextrin; sugars such as powdered sugar and lactose monohydrate; sugar alcohols such as lactose; D-mannitol; inorganic diluents such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, or combinations thereof. Diluents, also called “fillers”, increase the bulk of a tablet so that a practical size is provided for compression.

### Disintegrants

In some cases, a pharmaceutical composition described herein comprises a disintegrant. In some instances, the pharmaceutical composition is a solid oral pharmaceutical composition. Exemplary disintegrants include starches, alginic acid, crosslinked polymers such as, e.g., crosslinked polyvinylpyrrolidone, croscarmellose sodium, potassium or sodium starch glycolate, clays, celluloses, starches, gums, or combinations thereof. In some instances, disintegrants facilitate tablet disintegration after administration, or following contact with dissolution fluid, or as measured in an in vitro dissolution study.

### Glidants

In some cases, a pharmaceutical composition described herein comprises a glidant. In some instances, the pharmaceutical composition is a solid oral pharmaceutical composition. Exemplary glidants include silicon dioxide, talc, dried aluminum hydroxide gel, magnesium silicate, or combinations thereof.

### Lubricants

In some cases, a pharmaceutical composition described herein comprises lubricant. In some instances, the pharmaceutical composition is a solid oral pharmaceutical composition. Exemplary lubricants include magnesium stearate, calcium stearate, stearic acid, glyceryl behenate, polyethylene glycol, talc, or combinations thereof. In some instances, lubricants facilitate tablet manufacture.

### Gelling Agents

In some cases, a pharmaceutical compositions described herein comprises a gelling agent. In some instances, the pharmaceutical composition is a solid oral pharmaceutical composition. In some instances, gelling agents provide for an increased viscosity when a solid oral dosage form is crushed or ground and mixed in a fluid. The increased viscosity serves to discourage an abuser from tampering with the dosage form and thereafter injecting the tampered dosage form. In some instances, a gelling agent is released when the dosage form is tampered with and provides a gel-like quality to the tampered dosage form which slows the absorption of the opioid analgesic such that an abuser is less likely to obtain a rapid “high”. In some instances, when the dosage form is tampered with and exposed to a small amount (e.g., less than about 10 ml) of an aqueous liquid (e.g., water), the dosage form will be unsuitable for injection. Upon the addition of the aqueous liquid, in some instances, the tampered dosage form becomes thick and viscous, rendering it unsuitable for injection. The term “unsuitable for injection” means that one would have substantial difficulty injecting the dosage form (e.g., difficulty pushing the dosage form through a syringe) due to the viscosity imparted on the dosage form, thereby reducing the potential for abuse of the opioid analgesic in the dosage form. In some instances, the gelling agent is present in such an amount in the dosage form that attempts at evaporation (by the application of heat) to an aqueous mixture of the dosage form in an effort to produce a higher concentration of the therapeutic agent, produces a highly viscous substance unsuitable for injection.

In some cases, a pharmaceutical compositions described herein comprises a gelling agent. In some instances, the pharmaceutical composition is a solid oral pharmaceutical composition. In some instances, gelling agents include, for example and without limitation, sugars or sugar derived alcohols, such as mannitol, sorbitol, and the like, starch and starch derivatives, cellulose derivatives, such as microcrystalline cellulose, sodium carboxymethyl cellulose, methylcellulose, ethyl cellulose, hydroxyethyl cellulose, hydroxypropyl cellulose, hydroxypropyl methylcellulose, attapulgites, bentonites, dextrins, alginates, carrageenan, gum tragacanth, gum acacia, guar gum, xanthan gum, pectin, gelatin, kaolin, lecithin, magnesium aluminum silicate, carbomers, carbopols, polyvinylpyrrolidone, polyethylene glycol, polyethylene oxide, polyvinyl alcohol (PVA), silicon dioxide, and mixtures thereof. In some instances, the gelling agent is a polyalkylene oxide, such as polyethylene oxide or polypropylene oxide. In some instances, the gelling agent is polyethylene oxide. In some instances, the gelling agent is polyethylene oxide with an approximate molecular weight of about 100,000 to about 7,000,000 g/mol. In some instances, the gelling agent is polyethylene oxide with an approximate molecular weight of about 700,000 to about 5,000,000 g/mol. In some instances, the gelling agent is polyethylene oxide with an approximate molecular weight of about 7,000,000 g/mol.

In some cases, a pharmaceutical composition as described herein comprises between about 100% to about 90% by weight of microcrystalline cellulose. In some instances, the pharmaceutical composition comprises about 10%, 15%, 20%, 21%, 22%, 23%, 24%, 25%, 26%, 27%, 28%, 29%, 30%, 31%, 32%, 33%, 34%, 35%, 36%, 37%, 38%, 38.8%, 39%, 40%, 41%, 42%, 43%, 44%, 45%, 46%, 47%, 48%, 49%, 50%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, or 90% by weight of microcrystalline cellulose. In some instances, the pharmaceutical composition is a solid oral pharmaceutical composition.

In some cases, a pharmaceutical composition as described herein comprises between about 0.05% to about 15% by weight of croscarmellose sodium. In some instances, the pharmaceutical composition comprises about 0.05%, 0.1%, 0.5%, 1%, 2%, 3%, 3.5%, 3.6%, 4%, 5%, 6%, 7%, 8%, 9%, 10%, 11%, 12%, 13%, 14%, or 15% by weight of croscarmellose sodium. In some instances, the pharmaceutical composition is a solid oral pharmaceutical composition.

In some cases, a pharmaceutical composition as described herein comprises between about 0.05% to about 5% by weight of magnesium stearate. In some instances, the pharmaceutical composition comprises about 0.05%, 0.1%, 0.15%, 0.2%, 0.25%, 0.3%, 0.35%, 0.4%, 0.45%, 0.5%, 0.53%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.5%, 4%, 4.5%, or 5% by weight of magnesium stearate. In some instances, the pharmaceutical composition is a solid oral pharmaceutical composition.

In some cases, a pharmaceutical composition as described herein comprises between about 0.05% to about 10% by weight of hydroxypropyl methylcellulose. In some instances, the pharmaceutical composition comprises about 0.05%, 0.1%, 0.15%, 0.2%, 0.25%, 0.3%, 0.35%, 0.4%, 0.45%, 0.05%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.21%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.5%, 4%, 4.5%, 5%, 5.5%, 6%, 6.5%, 7%, 7.5%, 8%, 8.5%, 9%, 9.5%, or 10% by weight of hydroxypropyl methylcellulose. In some instances, the pharmaceutical composition is a solid oral pharmaceutical composition.

In some cases, a pharmaceutical composition as described herein comprises between about 0.05% to about 10% by weight of stearic acid. In some instances, the pharmaceutical composition comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.21%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.5%, 4%, 4.5%, 5%, 5.5%, 6%, 6.5%, 7%, 7.5%, 8%, 8.5%, 9%, 9.5%, or 10% by weight of stearic acid. In some instances, the pharmaceutical composition is a solid oral pharmaceutical composition.

In some cases, a pharmaceutical composition as described herein comprises between about 0.05% to about 10% by weight of sodium starch glycolate. In some instances, the pharmaceutical composition comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.21%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.5%, 4%, 4.5%, 5%, 5.5%, 6%, 6.5%, 7%, 7.5%, 8%, 8.5%, 9%, 9.5%, or 10% by weight of sodium starch glycolate. In some instances, the pharmaceutical composition is a solid oral pharmaceutical composition.

In some cases, a pharmaceutical composition as described herein comprises between about 10% to about 90% by weight of lactose monohydrate. In some instances, the pharmaceutical composition comprises about 10%, 15%, 20%, 21%, 22%, 23%, 24%, 25%, 26%. 27%, 28%, 29%, 30%, 31%, 32%, 33%, 34%, 35%, 36%, 37%, 38%, 38.8%, 39%, 40%, 41%, 42%, 43%, 44%, 45%, 46%, 47%, 48%, 49%, 50%, 60%, 65%, 70%, 71%, 72%, 73%, 74%, 75%, 76%, 77% 78%, 79%, 80%, 81%, 82%, 83%, 84%, 85%, 86%, 87%, 88%, 89%, or 90% by weight of lactose monohydrate. In some instances, the pharmaceutical composition is a solid oral pharmaceutical composition.

In some cases, a pharmaceutical composition as described herein comprises between about 10% to about 90% by weight of mannitol. In some instances, the pharmaceutical composition comprises about 10%, 15%, 20%, 21%, 22%, 23%, 24%, 25%, 26%, 27%, 28%, 29%, 30%, 32%, 33%, 34%, 35%, 36%, 37%, 38%, 38.8%, 39%, 40%, 41%, 42%, 43%, 44%, 45%, 46%, 47%, 48%, 49%, 50%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, or 90% by weight of mannitol. In some instances, the pharmaceutical composition is a solid oral pharmaceutical composition.

In sonic cases, an effective amount of active agents is mixed with a suitable pharmaceutically acceptable carrier. Such compositions are prepared according to methods known to those skilled in the art. The forms of the resulting compositions depend upon a variety of factors, including the intended mode of administration and the solubility of the compounds in the selected carrier or vehicle.

In some cases, dosage forms described herein are manufactured using processes that are well known to those of skill in the art. For example, for the manufacture of multi-layered solid compositions (e.g., bi-layered tablets, multi-layered tablets, or pills), the agents are dispersed uniformly in one or more excipients, for example, using high shear granulation, low shear granulation, fluid bed granulation, or by blending for direct compression.

### Matrices

### Outer Matrix

In sonic cases, a pharmaceutical composition described herein comprises an outer matrix. In some instances, the pharmaceutical composition is a solid oral pharmaceutical composition. In some instances, an outer matrix comprises one or more abuse deterrents. Non-limiting examples of abuse deterrent agents are gelling agents, nasal irritants, emetics, or a crush-deterrent agents. In some instances, an abuse deterrent agent is a gelling agent. In some instances, the gelling agent discourages an abuser from tampering with the dosage form and thereafter inhaling, injecting, and/or swallowing the tampered dosage form. In some instances, an outer matrix comprises a coating layer. In some instances, a coating layer comprises a gelling agent. In some instances, a coating layer further comprises a plasticizer. In some instances, a coating layer further comprises a glidant. In some instances, a coating layer further comprises a lubricant. In some instances, a coating layer further comprises a diluent. In some instances, a coating layer is in the form of a film coating, e.g., a glossy film, a pH independent film coating, an aqueous film coating, a dry powder film coating (e.g., complete dry powder film coating), or any combination thereof. In some instances, a coating layer is highly adhesive. In some instances, a coating layer provides low level of water permeation. In some instances, a coating layer provides oxygen barrier protection. In some instances, a coating layer is pigmented, clear, or white. In some instances, a coating layer is clear. Exemplary coating materials comprised in the coating layer include, without limitation, polyvinyl alcohol (PVA), cellulose acetate phthalate (CAP), polyvinyl acetate phthalate (PVAP), methacrylic acid copolymers, cellulose acetate trimellitate (CAT), hydroxypropyl cellulose, hydroxypropyl methylcellulose phthalate (HPIVICP), hydroxypropyl methylcellulose (HPMC), hydroxy propyl methyl cellulose acetate succinate (hypromellose acetate succinate), shellac, sodium alginate, zein, talc, polyethylene oxide, triethyl citrate, polyethylene glycol 400, polyethylene glycol 3350, and combination thereof. In some instances, the coating layer comprises a polyethylene oxide. In some instances, the coating layer comprises a polyethylene oxide with an approximate molecular weight of about 100,000 to about 7,000,000 g/mol. In some instances, the coating layer comprises a polyethylene oxide with an approximate molecular weight of about 700,000 to about 5,000,000 g/mol. In some instances, the coating layer comprises a polyethylene oxide with an approximate molecular weight of about 7,000,000 g/mol.

In some cases, a pharmaceutical composition described herein comprises a coating layer. In some instances, the pharmaceutical composition is a solid oral pharmaceutical composition. In some instances, a coating layer comprises about 40% to about 90% of an abuse deterrent, for example a gelling agent. In some instances, the coating layer comprises about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%, of an abuse deterrent, for example a gelling agent. In some instances, the gelling agent comprises polyethylene oxide. In some instances, the gelling agent comprises polyethylene oxide with an approximate molecular weight of about 100,000 to about 7,000,000 g/mol. In some instances, the gelling agent comprises polyethylene oxide with an approximate molecular weight of about 900,000 to about 5,000,000 g/mol. In some instances, the gelling agent comprises polyethylene oxide with an approximate molecular weight of about 7,000,000 g/mol.

In some cases, a pharmaceutical composition described herein comprises a coating layer. In some instances, the pharmaceutical composition is a solid oral pharmaceutical composition. In some instances, a coating layer comprises about 10 mg to about 100 mg of an abuse deterrent, for example a gelling agent, per tablet. In some instances, the coating layer comprises about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg of an abuse deterrent, for example a gelling agent, per tablet. In some instances, a gelling agent comprises polyethylene oxide. In some instances, the gelling agent comprises polyethylene oxide with an approximate molecular weight of about 100,000 to about 7,000,000 g/mol. In some instances, the gelling agent comprises polyethylene oxide with an approximate molecular weight of about 900,000 to about 5,000,000 g/mol. In some instances, the gelling agent comprises polyethylene oxide with an approximate molecular weight of about 7,000,000 g/mol.

In some cases, a pharmaceutical composition described herein comprises a coating layer. In some instances, the pharmaceutical composition is a solid oral pharmaceutical composition. In some instances, a coating layer comprises one or more abuse deterrent agents. In some instances, an abuse deterrent is subjected to appropriate processing to facilitate a coating procedure. For example, micronization, milling, jet-milling, or microfluidization of a gelling agent may be performed to produce particles of a desired size to facilitate a desired coating method. In some instances a particle size of approximately 10 μm is desirable. In some instances a particle size of less than 50 μm is desirable. In some instances a particle size of less than 80 μm is desirable. In some instances a particle size of less than 100 μm is desirable.

In some cases, a pharmaceutical composition described herein comprises a coating layer. In some instances, the pharmaceutical composition is a solid oral pharmaceutical composition. In some instances, a coating layer comprises an abuse deterrent agent and one or more excipients. In some instances, a coating layer comprises about 10% to about 40% of a lubricant. In some instances, the coating layer comprises about 10%, about 11%, about 12%, about 13%, about 14%, about 15%, about 15.5%, about 16%, about 16.5%, about 17%, about 17.5%, about 18%, about 18.5%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% of a lubricant. In some instances, the lubricant comprises hydroxypropyl cellulose. In some instances, the lubricant is hydroxypropyl cellulose. In some instances, a coating layer comprises about 1 mg to about 50 mg of a lubricant. In some instances, a coating layer comprises about 1 trig, about 2 mg, about 3 mg, about 4 mg, about 5 mg, about 6 mg, about 7 mg, about 8 mg, about 8.5 mg, about 9 mg, about 10 mg, about 11 mg, about 12 mg, about 13 mg, about 14 mg, about 15 mg, about 20 mg, about 25 mg, about 30 mg, about 35 mg, about 40 mg, about 45 mg, or about 50 mg of a lubricant. In some instances, the lubricant comprises hydroxypropyl cellulose. In some instances, the lubricant is hydroxypropyl cellulose.

In some cases, a pharmaceutical composition described herein comprises a coating layer. In some instances, the pharmaceutical composition is a solid oral pharmaceutical composition. In some instances, a coating layer comprises an abuse deterrent agent and one or more excipients. In some instances, the coating layer comprises about 0.5% to about 20% of a glidant. In some instances, the coating layer comprises about 0.5%, about 1%, about 2%, about 3%, about 4%, about 5%, about 6%, about 6.5%, about 7%, about 7.5%, about 8%, about 8.5%, about 8.8%, about 9%, about 9.5%, about 10%, about 11%, about 12%, about 13%, about 14%, about 15%, about 16%, about 17%, about 18%, about 19%, or about 20% of a glidant. In some instances, the glidant comprises talc. In some instances, the glidant is talc. In some instances, the talc is micronized. In some instances, the coating layer comprises about 0.5 mg to about 20 mg of a glidant. In some instances, the coating layer comprises about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, or about 20 mg of a glidant. In some instances, the glidant comprises talc. In some instances, the glidant is talc. In some instances, the talc is micronized.

In some cases, a pharmaceutical composition described herein comprises a coating layer. In some instances, the pharmaceutical composition is a solid oral pharmaceutical composition. In some instances, a coating layer comprises an abuse deterrent agent and one or more excipients. In some instances, the coating layer comprises about 0.05% to about 10% of a plasticizer. In some instances, the coating layer comprises about 0.05%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, about 5%, about 6%, about 7%, about 8%, about 9%, or about 10% of a plasticizer. In some instances, the plasticizer comprises polyethylene glycol 400, polyethylene glycol 3350, triethyl citrate, or combination thereof. In some instances, the coating layer comprises about 0.05 mg to about 10 mg of a plasticizer. In some instances, the coating layer comprises about 0.05 mg, about 0.1 mg, about 0.15 mg, about 0.2 mg, about 0.25 mg, about 0.3 mg, about 0.4 mg, about 0.5 mg, about 1 mg, about 1.1 mg, about 1.2 mg, about 1.3 mg, about 1.4 mg, about 1.5 mg, about 1.6 mg, about 1.7 mg, about 1.8 trig, about 1.9 mg, about 2 mg, about 2.5 mg, about 3 mg, about 3.5 mg, about 4 mg, about 4.5 mg, about 5 mg, about 6 mg, about 7 mg, about 8 mg, about 9 mg, or about 10 mg of a plasticizer. In some instances, the plasticizer comprises polyethylene glycol 400, polyethylene glycol 3350, triethyl citrate, or combination thereof.

In some cases, a pharmaceutical composition described herein comprises a coating layer. In some instances, the pharmaceutical composition is a solid oral pharmaceutical composition. In some instances, the coating layer has a total weight of about 1 mg to about 100 mg. In some instances, the coating later has a total weight of about 1 mg, about 5 mg, about 10 mg, about 15 mg, about 20 mg, about 25 mg, about 30 mg, about 35 mg, about 40 mg, about 45 mg, about 50 trig, about 55 mg, about 60 mg, about 65 mg, about 70 mg, about 75 mg, about 80 mg, about 85 mg, about 90 mg, about 95 mg, or about 100 mg per tablet. In some instances, the coating later has a total weight of less than about 1 mg, less than about 5 mg, less than about 10 mg, less than about 15 mg, less than about 20 mg, less than about 25 mg, less than about 30 mg, less than about 35 mg, less than about 40 mg, less than about 45 mg, less than about 50 mg, less than about 55 mg, less than about 60 mg, less than about 65 mg, less than about 70 mg, less than about 75 mg, less than about 80 mg, less than about 85 mg, less than about 90 mg, less than about 95 mg, or less than about 100 mg.

In some cases, a pharmaceutical composition described herein comprises a coating layer. In some instances, the pharmaceutical composition is a solid oral pharmaceutical composition. In some instances, the coating layer is about 1% to about 20% of the weight of the pharmaceutical composition. In some instances, the coating layer is about 10% to about 15% of the weight of the pharmaceutical composition. In some instances, the coating layer is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% of the weight of the pharmaceutical composition. In some instances, the coating layer is greater than about 1%, greater than about 2%, greater than about 3%, greater than about 4%, greater than about 5%, greater than about 6%, greater than about 7%, greater than about 8%, greater than about 9%, greater than about 10%, greater than about 11%, greater than about 12%, greater than about 13%, greater than about 14%, greater than about 15%, greater than about 16%, greater than about 17%, greater than about 18%, or greater than about 19% of the weight of the pharmaceutical composition. In some instances, the coating layer is no more than about 1%, no more than about 2%, no more than about 3%, no more than about 4%, no more than about 5%, no more than about 6%, no more than about 7%, no more than about 8%, no more than about 9%, no more than about 10%, no more than about 11%, no more than about 12%, no more than about 13%, no more than about 14%, no more than about 15%, no more than about 16%, no more than about 17%, no more than about 18%, no more than about 19%, no more than about 20% of the weight of the pharmaceutical composition.

In some instances, a coating described herein is manufactured using processes that are well known to those of skill in the art. For example, the coating solution is prepared using microfluidization or other suitable micronizati on techniques. In some instances, a coating described herein is anhydrous and ethanol based.

### Sub-Outer Matrix

In some cases, a pharmaceutical composition disclosed herein comprises a sub-outer matrix. In some instances, the pharmaceutical composition is a solid oral pharmaceutical composition. In some instances, a sub-outer matrix is a sub-coating layer. In some instances, the sub-coating layer is between a core and a coating layer. In some instances, the sub-coating layer is between a core and an outer matrix. In some instances, the sub-layer is between an out matrix and an inner matrix. In some instances, the sub-layer is between an outer matrix and two or more inner matrices. In some instances, the sub-coating layer provides for improved adhesion of the coating layer to the core. In some instances, the sub-coating layer provides for improved disintegration rate and/or dissolution rate of the core. In some instances, the sub-coating layer comprises at least one pharmaceutically acceptable excipient. In some instances, the pharmaceutically acceptable excipient comprises a polymer. In some instances, the polymer is a cellulosic polymer. In some instances, the pharmaceutically acceptable excipient comprises hydroxypropyl methylcellulose (HPMC). In some instances, the pharmaceutically acceptable excipient is HPMC.

In some cases, a sub-coating layer described herein is about 1% to about 10% of the weight of the pharmaceutical composition. In some instances, the sub-coating layer is about 1%, about 2%, about 3%, about 4%, about 5%, about 6%, about 7%, about 8%, about 9%, or about 10% of the weight of the pharmaceutical composition. In some instances, the sub-coating layer is greater than about 1%, greater than about 2%, greater than about 3%, greater than about 4%, greater than about 5%, greater than about 6%, greater than about 7%, greater than about 8%, or greater than about 9% of the weight of the pharmaceutical composition. In some instances, the sub-coating layer is no more than about 1%, no more than about 2%, no more than about 3%, no more than about 4%, no more than about 5%, no more than about 6%, no more than about 7%, no more than about 8%, no more than about 9%, or no more than about 10% of the weight of the pharmaceutical composition. In some instances, the sub-coating layer comprises HPMC and provides a weight gain of about 1% to about 5%. In some instances, the sub-coating layer comprises HPMC and provides a weight gain of about 3%.

In some cases, a sub-coating layer described herein is present in an amount of about 1 mg to about 20 mg. In some instances, the sub-coating layer is present in an amount of about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, or about 20 mg. In some instances, the sub-coating layer comprises HPMC.

### Timed-Release

In some cases, a pharmaceutical composition described herein comprises a controlled-release formulation. In some instances, the pharmaceutical composition is a solid oral pharmaceutical composition. In some instances, controlled-release formulations comprise one or more combination of excipients that slow the release of the agents by coating or temporarily bonding or decreasing their solubility of the active agents. Examples of these excipients include cellulose ethers such as hydroxypropyl methylcellulose or microcrystalline cellulose; polyvinyl acetate-based excipients; polymers and copolymers based on methacrylates and methacrylic acid; croscarmellose sodium, magnesium stearate or stearic acid.

In some cases, a pharmaceutical composition described herein comprises an immediate-release formulation. In some instances, the pharmaceutical composition is a solid oral pharmaceutical composition. In some instances, immediate-release formulations comprise one or more combinations of excipients that allow for a rapid release of the pharmaceutically active agent. In some instances, immediate-release excipients are hydroxypropyl methylcellulose, microcrystalline cellulose, sodium carboxymethyl cellulose, sodium starch glycolate, corn starch, colloidal silica, sodium laurel sulphate, magnesium stearate, stearic acid, croscarmellose sodium, crospovidone NF, Avicel PH200, or combinations thereof.

In some cases, a pharmaceutical composition described herein is formulated for oral delivery to a subject in need. In some instances, the pharmaceutical composition is a solid oral pharmaceutical composition. In some instances, the pharmaceutical composition is in the form of a tablet. In some instances, the pharmaceutical composition is formulated so as to deliver two pharmaceutically active agents to a subject through a mucosa layer in the mouth or esophagus. In some instances, the composition is formulated to deliver two pharmaceutically active agents to a subject through a mucosa layer in the stomach and/or intestines.

In some cases, a pharmaceutical composition described herein is provided in modified release dosage forms. In some instances, the pharmaceutical composition is a solid oral pharmaceutical composition. Suitable modified release dosage vehicles include, but are not limited to, hydrophilic or hydrophobic matrix devices, water-soluble separating layer coatings, enteric coatings, osmotic devices, multi-particulate devices, and combinations thereof. In some instances, the compositions also comprise non-release controlling excipients.

In some cases, a pharmaceutical composition described herein is provided in enteric coated dosage forms. In some instances, the pharmaceutical composition is a solid oral pharmaceutical composition. In some instances, the enteric coated dosage forms comprise non-release controlling excipients. In some instances, the compositions are in the form of enteric-coated granules, as controlled-release capsules for oral administration. In some instances, the compositions further comprise cellulose, disodium hydrogen phosphate, hydroxypropyl cellulose, hypromellose, lactose, mannitol, or sodium lauryl sulfate. In some instances, the compositions are in the form of enteric-coated pellets, as controlled-release capsules for oral administration. In some instances, the compositions further comprise glycerol monostearate hydroxypropyl cellulose, hypromellose, magnesium stearate, methacrylic acid copolymer type C, polysorbate 80, sugar spheres, talc, triethyl citrate, or any combination thereof.

In some cases, a pharmaceutical composition described herein is enteric-coated controlled-release tablets for oral administration. In some instances, the pharmaceutical composition is a solid oral pharmaceutical composition. In some instances, the compositions further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, yellow ferric oxide, or any combination thereof.

In some cases, a pharmaceutical composition described herein further comprises calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, triethyl citrate, or any combination thereof. A pharmaceutical composition can be a solid oral pharmaceutical composition.

In some cases, a pharmaceutical composition described herein is formulated as a modified release dosage form, including immediate-, delayed-, extended-, prolonged-, sustained-, pulsatile-, controlled-, extended, accelerated- and fast-, targeted-, programmed-release, and gastric retention dosage forms. In some instances, the pharmaceutical composition is a solid oral pharmaceutical composition.

### Inner Matrices

In some cases, a pharmaceutical composition described herein comprises an outer matrix. In some instances, a pharmaceutical composition described herein comprises one or more inner matrices surrounded by an outer matrix. In some instances, the pharmaceutical composition is a solid oral pharmaceutical composition. In some instances, a first inner matrix is surrounded by an outer matrix while a second inner matrix is not surrounded by the outer matrix. In some instances, at least one of the one or more inner matrices comprises an opioid analgesic. In some instances, at least one of the one or more inner matrices comprises a non-opioid analgesic. In some instances, at least one of the one or more inner matrices comprises an antiemetic. In some instances, at least one of the one or more inner matrices comprises an opioid analgesic and an anti-emetic. In some instances, the at least one of the one or more inner matrices comprises an opioid analgesic, a non-opioid analgesic, and an anti-emetic. In some instances, one or more inner matrices comprises an inner core. In some instances, the total weight of the inner core is about 50 mg to about 1000 mg. In some instances, the total weight of the inner core is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 100 mg. In some instances, the opioid analgesic is oxycodone or a pharmaceutically acceptable salt thereof. In some instances, the opioid analgesic is oxycodone hydrochloride. In some instances, the opioid analgesic is hydrocodone or a pharmaceutically acceptable salt thereof. In some instances, the opioid analgesic is hydrocodone bitartrate. In some examples, the non-opioid analgesic is acetaminophen. In some instances, the anti-emetic is promethazine or a pharmaceutically acceptable salt thereof. In some instances, the anti-emetic is promethazine hydrochloride.

In some cases, a core as described herein comprises about 0.5%, 0.6%, 0.7%, 0.8%, 0.9%, 1%, 1.1%, 1.2%, 1.2%, 1.3%, 1.4%, 1.5%, 1.6%, 1.7%, 1.8%, 1.9%, 2%, 2.1%, 2.2%, 2.2%, 7.3%, 2.4%, 2.5%, 2.6%, 2.7%, 2.8%, 2.9%, 3%, 3.1%, 3.2%, 3.2%, 3.3%, 3.4%, 3.5%, 3.6%, 3.7%, 3.8%, 3.9%, 4%, 4.1%, 4.2%, 4.2%, 4.3%, 4.4%, 4.5%, 4.6%, 4.7%, 4.8%, 4.9%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 16%, 17%, 18%, 19%, or 20% by weight of an opioid analgesic. In some instances, the opioid analgesic is oxycodone or a pharmaceutically acceptable salt thereof. In some instances, the oxycodone or a pharmaceutically acceptable salt thereof is oxycodone hydrochloride. In some instances, the opioid analgesic is hydrocodone or a pharmaceutically acceptable salt thereof. In some instances, the hydrocodone or a pharmaceutically acceptable salt thereof is hydrocodone bitartrate.

In some cases, a core as described herein comprises between about 0.1% to about 20% by weight of an antiemetic. In some instances, the pharmaceutical composition comprises about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 3%, 4%, 5%, 6%, 7%, 8%, 9%, 10%, 11%, 12%, 13%, 14%, 15%, 16%, 17%, 18%, 19%, or 20% by weight of an antiemetic. In some instances, the antiemetic is promethazine or a pharmaceutically acceptable salt thereof. In some instances, the promethazine or a pharmaceutically acceptable salt thereof is promethazine hydrochloride.

In some cases, a core as described herein comprises between about 30% to about 70% by weight of a non-opioid analgesic. In some instances, a pharmaceutical composition comprises 30%, 31%, 32%, 33%, 34%, 35%, 36%, 37%, 38%, 39%, 40%, 41%, 42%, 43%, 44%, 45%, 46%, 47%, 48%, 49%, 50%, 51%, 51.5%, 51.6%, 52%, 53%, 54%, 55%, 56%, 57%, 58%, 59%, 60%, 61%, 62%, 63%, 64%, 65%, 66%, 67%, 68%, 69%, or 70% by weight of a non-opioid analgesic. In some instances, the non-opioid analgesic is acetaminophen.

In some cases, a core as described herein comprises between about 10% to about 90% by weight of microcrystalline cellulose. In some instances, the pharmaceutical composition comprises about 10%, 15%, 20%, 21%, 22%, 23%, 24%, 25%, 26%, 27%, 28%, 29%, 30%, 31%, 32%, 33%, 34%, 35%, 36%, 37%, 38%, 38.8%, 39%, 40%, 41%, 42%, 43%, 44%, 45%, 46%, 47%, 48%, 49%, 50%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, or 90% by weight of microcrystalline cellulose.

In some cases, a core as described herein comprises between about 0.05% to about 5% by weight of croscarmellose sodium. In some instances, the pharmaceutical composition comprises about 0.05%, 0.1%, 0.5%, 1%, 2%, 3%, 3.5%, 3.6%, 4%, 5%, 6%, 7%, 8%, 9%, 10%, 11%, 12%, 13%, 14%, or 15% by weight of croscarmellose sodium.

In some cases, a core as described herein comprises between about 0.05% to about 5% by weight of magnesium stearate. In some instances, the pharmaceutical composition comprises about 0.05%, 0.1%, 0.15%, 0.2%, 0.25%, 0.3%, 0.35%, 0.4%, 0.45%, 0.5%, 0.53%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.5%, 4%, 4.5%, or 5% by weight of magnesium stearate.

In some cases, a core as described herein comprises between about 0.05% to about 10% by weight of hydroxypropyl methylcellulose. In some instances, the pharmaceutical composition comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.21%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.5%, 4%, 4.5%, 5%, 5.5%, 6%, 6.5%, 7%, 7.5%, 8%, 8.5%, 9%, 9.5%, or 10% by weight of hydroxypropyl methylcellulose.

In some cases, a core as described herein comprises between about 0.05% to about 10% by weight of stearic acid. In some instances, the pharmaceutical composition comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.21%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.5%, 4%, 4.5%, 5%, 5.5%, 6%, 6.5%, 7%, 7.5%, 8%, 8.5%, 9%, 9.5%, or 10% by weight of stearic acid.

In some cases, a core as described herein comprises between about 0.05% to about 10% by weight of sodium starch glycolate. In some instances, the pharmaceutical composition comprises about 0.0.5%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 21%, 2.15%, 2.2%, 2.21%, 2.25%, 2.3%, 2.35%, 2.4%, 2.45%, 2.5%, 2.55%, 2.6%, 2.65%, 2.7%, 2.75%, 2.8%, 2.85%, 7.9%, 2.95%, 3%, 3.5%, 4%, 4.5%, 5%, 5.5%, 6%, 6.5%, 7%, 7.5%, 8%, 8.5%, 9%, 9.5%, or 10% by weight of sodium starch glycolate.

In some cases, a core as described herein comprises between about 10% to about 90% by weight of lactose monohydrate. In some instances, the pharmaceutical composition comprises about 10%, 15%, 20%, 21%, 22%, 23%, 24%, 25%, 26%, 27%, 28%, 29%, 30%, 31%, 32%, 33%, 34%, 35%, 36%, 37%, 38%, 38.8%, 39%, 40%, 41%, 42%, 43%, 44%, 45%, 46%, 47%, 48%, 49%, 50%, 60%, 65%, 70%, 71%, 77%, 73%, 74%, 75%, 76%, 77%, 78%, 79%, 80%, 81%, 82%, 83%, 84%, 85%, 86%, 87%, 88%, 89%, or 90% by weight of lactose monohydrate.

In some cases, a core as described herein comprises between about 10% to about 90% by weight of mannitol. In some instances, the pharmaceutical composition comprises about 10%, 15%, 20%, 21%, 22%, 23%, 24%, 25%, 26%, 27%, 28%, 29%, 30%, 31%, 32%, 33%, 34%, 35%, 36%, 37%, 38%, 38.8%, 39%, 40%, 41%, 42%, 43%, 44%, 45%, 46%, 47%, 48%, 49%, 50%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, or 90% by weight of mannitol.

In some cases, a pharmaceutical composition described herein comprises one or more inner matrices. In some instances, one or more inner matrices can comprise at least two inner layers. In some instances, a pharmaceutical composition described herein comprises a first inner matrix and a second inner matrix. In some instances, a first inner matrix comprises a first layer and a second inner matrix comprises a second layer. In some instances, a pharmaceutical composition described herein comprises two layers (a first layer and a second layer) in a bi-layered tablet. In some instances, the pharmaceutical composition is a solid oral pharmaceutical composition.

In some cases, a pharmaceutical composition described herein comprises at least two layers. In some instances, a first layer in a pharmaceutical composition described herein comprises one or more excipients, including but not limited to microcrystalline cellulose, silicified microcrystalline cellulose, croscarmellose sodium, hydroxypropyl methylcellulose, stearic acid, magnesium stearate, sodium starch glycolate, lactose monohydrate, mannitol, or any combination thereof. In some instances, the microcrystalline cellulose is silicified microcrystalline cellulose. In some instances, a first layer further comprises an opioid analgesic. In some instances, a first layer thriller comprises a non-opioid analgesic. in some instances, a first layer further comprises an opioid analgesic and a non-opioid analgesic. In some instances, the opioid analgesic is oxycodone or a pharmaceutically acceptable salt thereof. In some instances, the opioid analgesic is oxycodone hydrochloride, in some instances, the opioid analgesic is hydrocodone or a pharmaceutically acceptable salt thereof. In some instances, the opioid analgesic is hydrocodone bitartrate. in some instances, the non-opioid analgesic is acetaminophen or a pharmaceutically acceptable salt thereof. A pharmaceutical composition can be a solid oral pharmaceutical composition.

In some cases, a pharmaceutical composition described herein comprises at least two layers. In some instances, a first layer in a pharmaceutical composition described herein comprises about 0.5 mg to about 100 mg of an opioid analgesic. In some instances, a first layer in a pharmaceutical composition described herein comprises about 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, or 100 mg of an opioid analgesic. In some instances, a first layer in a pharmaceutical composition described herein comprises about 0.5 mg to about 100 mg of oxycodone or a pharmaceutically acceptable salt thereof. In some instances, a first layer in a pharmaceutical composition described herein comprises about 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, or 100 mg of oxycodone or a pharmaceutically acceptable salt thereof. In some instances, a first layer in a pharmaceutical composition described herein comprises about 0.5 mg to about 100 mg of oxycodone hydrochloride. In some instances, a first layer in a pharmaceutical composition described herein comprises about 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, or 100 mg of oxycodone hydrochloride. In some instances, the pharmaceutical composition is a solid oral pharmaceutical composition.

In some cases, a pharmaceutical composition described herein comprises at least two layers. In some instances, a first layer in a pharmaceutical composition described herein comprises about 0.5 mg to about 100 mg of an opioid analgesic. In some instances, a first layer in a pharmaceutical composition described herein comprises about 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 1.9 mg, 19.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, or 100 mg of an opioid analgesic. In some instances, a first layer in a pharmaceutical composition described herein comprises about 0.5 mg to about 100 mg of hydrocodone or a pharmaceutically acceptable salt thereof. In some instances, a first layer in a pharmaceutical composition described herein comprises about 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, or 1.00 mg of hydrocodone or a pharmaceutically acceptable salt thereof. In some instances, a first layer in a pharmaceutical composition described herein comprises about 0.5 mg to about 100 mg of hydrocodone bitartrate. In some instances, a first layer in a pharmaceutical composition described herein comprises about 0.5 mg, 1 mg, 1.5 mg, 2 mg, mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 11.2.5 mg, 13 mg, 13.5 mg, 14 mg, 11.4.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, or 100 mg of hydrocodone bitartrate. A pharmaceutical composition can be a solid oral pharmaceutical composition.

In some cases, a pharmaceutical composition described herein comprises at least two layers. In some instances, a first layer in a pharmaceutical composition described herein comprises about 200 mg to about 600 mg of a non-opioid analgesic. In some instances, a first layer in a pharmaceutical composition described herein comprises about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, or about 650 mg of a non-opioid analgesic. In some instances, a first layer in a pharmaceutical composition described herein comprises about 200 mg to about 600 mg of acetaminophen. In some instances, a first layer in a pharmaceutical composition described herein comprises about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg. about 525 mg. about 550 mg, about 575 mg, about 600 mg, about 625 mg, or about 650 mg of acetaminophen. In some instances, the pharmaceutical composition is a solid oral pharmaceutical composition.

In some cases, a pharmaceutical composition described herein comprises at least two layers. In some instances, a first layer in a pharmaceutical composition described herein comprises about 5 mg to about 250 mg of microcrystalline cellulose. In some instances, a first layer in a pharmaceutical composition described herein comprises about 5 mg, 10 mg, 15 mg, 16 mg 17 mg, 18 mg, 19 mg, 20 mg, 21 mg, 22 mg, 23 mg, 24 mg, 25 mg, 26 mg, 27 mg, 28 mg, .29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg, 100 mg, 110 mg, 120 mg, 130 mg 140 mg 141 mg, 142 mg 143 mg, 144 mg, 145 mg, 146 mg, 147 mg, 148 mg, 149 mg, 150 mg, 150.4 mg, 151 mg, 152 mg, 153 mg, 154 mg, 155 mg, 157 mg, 158 mg, 159 mg, 160 mg, 170 mg, 180 mg, 190 mg, 200 mg, 210 mg, 220 mg, 230 mg, 240 mg, or 250 mg of microcrystalline cellulose. In some instances, the pharmaceutical composition is a solid oral pharmaceutical composition.

In some cases, a pharmaceutical composition described herein comprises at least two layers. In some instances, a first layer in a pharmaceutical composition described herein comprises about 0.2 mg to about 25 mg of hydroxypropyl methylcellulose. In some instances, a first layer in a pharmaceutical composition described herein comprises about 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 6 mg, 7 mg, 8 mg, 9 mg, 10 mg, 10.5 mg, 11 mg, 11.5 mg, 12 mg, 12.5 mg, .13 mg, 13.5 mg, 14 mg, 14.5 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.5 mg, 17 mg, 17.5 mg, 18 mg, 18.5 mg, 19 mg, 19.5 mg, 20 mg, 21 mg, 22 mg, 23 mg, 24 mg, or 25 mg of hydroxyprorₛ71 methylcellulose. In some instances, the pharmaceutical composition is a solid oral pharmaceutical composition.

In some cases, a pharmaceutical composition described herein comprises at least two layers. In some instances, a first layer in a pharmaceutical composition described herein comprises about 0.05 mg to about 5 mg of magnesium stearate. In some instances, a first layer in a pharmaceutical composition described herein comprises about 0.05 mg, 0.1 mg, 0.115 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.25 mg, 3.5 mg, 4 mg, 4.5 mg, or 5 mg of magnesium stearate. In some instances, the pharmaceutical composition is a solid oral pharmaceutical composition.

In some cases, a pharmaceutical composition described herein comprises at least two layers. In some instances, a first layer in a pharmaceutical composition described herein comprises about 0.05 mg to about 5 mg of stearic acid. In some instances, a first layer in a pharmaceutical composition described herein comprises about 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.25 mg, 3.5 mg, 4 mg, 4.5 mg, or 5 mg of stearic acid. In some instances, the pharmaceutical composition is a solid oral pharmaceutical composition.

In some cases, a pharmaceutical composition described herein comprises at least two layers. in some instances, a first layer in a pharmaceutical composition described herein comprises about 0.2 mg to about 5 mg of sodium starch glycolate. In some instances, a first layer in a pharmaceutical composition described herein comprises about 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg or 5 mg of sodium starch glycolate. In some instances, the pharmaceutical composition is a solid oral pharmaceutical composition.

In some cases, a pharmaceutical composition described herein comprises at least two layers. In some instances, a first layer in a pharmaceutical composition described herein comprises about 1 mg to about 20 mg of croscarmellose. In some instances, a first layer in a pharmaceutical composition described herein comprises about 0.5 mg, 1 mg, 1.5 mg, 2 mg, 2.5 mg, 3 mg, 3.5 mg, 4 mg, 4.5 mg, 5 mg, 5.5 mg, 6 mg. 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.5 mg, 17 mg, 17.5 mg, 18 mg, 18.5 mg, 19 mg, 19.5 mg, or 20 mg of croscarmellose sodium. In some instances, the pharmaceutical composition is a solid oral pharmaceutical composition.

In some cases, a pharmaceutical composition described herein comprises at least two layers. in some instances, a first layer in a pharmaceutical composition described herein comprises about 10 mg to about 100 mg of lactose monohydrate. In some instances, a first layer in a pharmaceutical composition described herein comprises about 10 mg, 15 mg, 20 mg, 25 mg, 30 mg, 35 mg, 40 mg, 45 mg, 50 mg, 55 mg, 56 mg, 57 mg, 58 mg, 58.5 mg, 58.6 mg, 58.67 mg, 59 mg, 60 mg, 65 mg, 70 mg, 75 mg, 80 mg, 85 mg, 90 mg, 95 mg, or 100 mg of lactose monohydrate. In some instances, the pharmaceutical composition is a solid oral pharmaceutical composition.

In some cases, a pharmaceutical composition described herein comprises at least two layers. In some instances, a first layer in a pharmaceutical composition described herein comprises about 10 mg to about 100 mg of mannitol. In some instances, a first layer in a pharmaceutical composition described herein comprises about 10 mg, 15 mg, 20 mg, 25 mg, 30 mg, 35 mg, 40 mg, 45 mg, 50 mg, 55 mg, 56 mg, 57 mg, 58 mg, 58.5 mg, 58.6 mg, 58.67 mg, 59 mg, 60 mg, 65 mg, 70 mg, 75 mg, 80 mg, 85 mg, 90 mg, 95 mg, or 100 mg of mannitol. In some instances, the pharmaceutical composition is a solid oral pharmaceutical composition.

In some cases, a first layer in a pharmaceutical composition described herein comprises from about 15 mg to about 50 mg of microcrystalline cellulose, from about 0.2 mg to about 5 mg of hydroxypropyl methylcellulose, from about 0.05 mg to 3 mg of magnesium stearate, from about 0.05 mg to about 3 mg of stearic acid, and from about 0.2 mg to about 5 mg of sodium starch glycolate. In some instances, the first layer comprises from about 15 mg to about 50 mg of microcrystalline cellulose, from about 0.2 mg to about 5 mg of hydroxypropyl methylcellulose, from about 0.05 mg to 3 mg of magnesium stearate, and from about 0.05 mg to about 3 mg of stearic acid. In some instances, the first layer comprises one or more additional pharmaceutically acceptable excipients, salts, and/or carriers disclosed herein. In some instances, a first layer further comprises an opioid analgesic. In some instances, the opioid analgesic is present in an amount of about 0.5 mg to about 20 mg. In some instances, the opioid analgesic is oxycodone or a pharmaceutically acceptable salt thereof. in some instances, the opioid analgesic is oxycodone hydrochloride, in some instances, a pharmaceutical composition described herein comprises at least two layers. In some instances, a first layer in a pharmaceutical composition described herein comprises about 5 mg of oxycodone hydrochloride, about 27.65 mg of microcrystalline cellulose, about 1 mg of hydroxypropyl methylcellulose, about 0.175 mg of magnesium stearate, about 0.175 mg of stearic acid, and about 1 mg of sodium starch glycolate. In some instances, a first layer in a pharmaceutical composition described herein comprises about 5 mg of oxycodone hydrochloride, about 27.15 mg of microcrystalline cellulose, about 2 mg of hydroxypropyl methylcellulose, about 0.175 mg of magnesium stearate, about 0.175 mg of stearic acid, and about 0.5 mg of sodium starch glycolate. In some instances, the pharmaceutical composition is a solid oral pharmaceutical composition.

In some cases, a first layer in a pharmaceutical composition described herein comprises from about 50 mg to about 200 mg of microcrystalline cellulose, from about 1 mg to about 20 mg of hydroxypropyl methylcellulose, from about 0.05 mg to 3 mg of magnesium stearate, from about 0.05 mg to about 3 mg of stearic acid, from about 1 mg to about 15 mg of croscarmellose sodium, and from about 10 mg to about 100 mg of lactose monohydrate. In some instances, the first layer comprises from about 50 mg to about 200 mg of microcrystalline cellulose, from about 1 mg to about 20 mg of hydroxypropyl methylcellulose, from about 0.05 mg to 3 mg of magnesium stearate, and from about 0.05 mg to about 3 mg of stearic acid. In some instances, the first layer comprises one or more additional pharmaceutically acceptable excipients, salts, and/or carriers disclosed herein. In some instances, a first layer further comprises an opioid analgesic. In some instances, the opioid analgesic is present in an amount of about 0.5 mg to about 20 mg. In some instances, the opioid analgesic is oxycodone or a pharmaceutically acceptable salt thereof. In some instances, the opioid analgesic is oxycodone hydrochloride. In some instances, a pharmaceutical composition described herein comprises at least two layers. In some instances, a first layer in a pharmaceutical composition described herein comprises about 5 mg of oxycodone hydrochloride, about 6.67 mg of croscarmellose sodium, about 117.33 mg of microcrystalline cellulose, about 10.33 mg of hydroxypropyl methylcellulose, about 1 mg of magnesium stearate, about 1 mg of stearic acid, and about 58.67 mg of lactose monohydrate. In some instances, a first layer in a pharmaceutical composition described herein comprises about 5 mg of oxycodone hydrochloride, about 6.67 mg of croscarmellose sodium, about 120.49 mg of microcrystalline cellulose, about 5.6 mg of hydroxypropyl methylcellulose, about 1 mg of magnesium stearate, about 1 mg of stearic acid, and about 60.24 mg of lactose monohydrate. In some instances, the pharmaceutical composition is a solid oral pharmaceutical composition.

In some cases, a first layer in a pharmaceutical composition described herein comprises from about 50 mg to about 200 mg of microcrystalline cellulose, from about 1 mg to about 20 mg of hydroxypropyl methylcellulose, from about 0.05 mg to 3 mg of magnesium stearate, from about 0.05 mg to about 3 mg of stearic acid, from about 1 mg to about 15 mg of croscarmellose sodium, and from about 10 mg to about 100 mg of mannitol. In some instances, the first layer comprises from about 50 mg to about 200 mg of microcrystalline cellulose, from about 1 mg to about 20 mg of hydroxypropyl methylcellulose, from about 0.05 mg to 3 mg of magnesium stearate, and from about 0.05 mg to about 3 mg of stearic acid. in some instances, the first layer comprises one or more additional pharmaceutically acceptable excipients, salts, and/or carriers disclosed herein. In some instances, a first layer further comprises an opioid analgesic. In some instances, the opioid analgesic is present in an amount of about 0.5 mg to about 20 mg. In some instances, the opioid analgesic is oxycodone or a pharmaceutically acceptable salt thereof. In some instances, the opioid analgesic is oxycodone hydrochloride. In some instances, a pharmaceutical composition described herein comprises at least two layers. In some instances, a first layer in a pharmaceutical composition described herein comprises about 5 mg of oxycodone hydrochloride, about 6.67 mg of croscarmellose sodium, about 117.33 mg of microcrystalline cellulose, about 10.33 mg of hydroxypropyl methylcellulose, about 1 mg of magnesium stearate, about 1 mg of stearic acid, and about 58.67 mg of mannitol. In some instances, a first layer in a pharmaceutical composition described herein comprises about 5 mg of oxycodone hydrochloride, about 6.67 mg of croscarmellose sodium, about 120.49 mg of microcrystalline cellulose, about 5.6 mg of hydroxypropyl methylcellulose, about 1 mg of magnesium stearate, about 1 mg of stearic acid, and about 60.24 mg of mannitol. In some instances, the pharmaceutical composition is a solid oral pharmaceutical composition.

In some cases, a first layer in a pharmaceutical composition described herein comprises from about 100 mg to about 250 mg of microcrystalline cellulose, from about 5 mg to about 20 mg of croscarrnellose sodium, from about 5 mg to about 25 mg of hydroxypropyl methylcellulose, from about 0.5 mg to 5 mg of magnesium stearate, from about 0.5 mg to about 5 mg of stearic acid. In some instances, the first layer comprises one or more additional pharmaceutically acceptable excipients, salts, and/or carriers disclosed herein. In some instances, a first layer further comprises an opioid analgesic. In some instances, the opioid analgesic is present in an amount of about 2.5 mg to about 50 mg. In some instances, the opioid analgesic is hydrocodone or a pharmaceutically acceptable salt thereof. In some instances, the opioid analgesic is hydrocodone bitartrate. In some instances, a first layer further comprises a non-opioid analgesic. In some instances, the non-opioid analgesic is present in an amount of about 200 mg to about 600 mg. In some instances, the non-opioid analgesic is acetaminophen. In some instances, a pharmaceutical composition described herein comprises at least two layers. In some instances, a first layer in a pharmaceutical composition described herein comprises about 7.5 mg of hydrocodone bitartrate, about 325 mg of acetaminophen, about 150.4 mg of microcrystalline cellulose, about 15.5 mg of hydroxypropyl methylcellulose, about 2.75 mg of magnesium stearate, about 2.75 mg of stearic acid, and about 10 mg of croscarmellose sodium. In some instances, a first layer in a pharmaceutical composition described herein comprises about 7.5 mg of hydrocodone bitartrate, about 150.4 mg of microcrystalline cellulose, about 15.5 mg of hydroxyprorₛ71 methylcellulose, about 2.75 mg of magnesium stearate, about 2.75 mg of stearic acid, and about 10 mg of croscarmellose sodium. In some instances, the pharmaceutical composition is a solid oral pharmaceutical composition.

In some cases, a pharmaceutical composition described herein comprises at least two layers. In some instances, a first layer in a pharmaceutical composition described herein comprises between about 0.1% to about 45% by weight of an opioid analgesic. In some instances, the first layer comprises about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, ^{2.5}%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% by weight of an opioid analgesic. In some instances, the opioid analgesic is oxycodone or a pharmaceutically acceptable salt thereof. In some instances, the opioid analgesic is oxycodone hydrochloride. in some instances, the opioid analgesic is hydrocodone or a pharmaceutically acceptable salt thereof. In some instances, the opioid analgesic is hydrocodone bitartrate. In some instances, the pharmaceutical composition is a solid oral pharmaceutical composition.

In some cases, a pharmaceutical composition described herein comprises at least two layers. In some instances, a first layer in a pharmaceutical composition described herein comprises between about 20% to about 80% by weight of a non-opioid analgesic. In some instances, the first layer comprises about 20%, 25%, 30%, 35%, 40%, 45%, 50%, 51%, 52%, 53%, 54%, 55%, 56%, 57%, 58%, 59%, 60%, 61%, 62%, 63%, 64%, 65%, 65.5%, 65.7%, 66%, 67%, 68%, 69%, 70%, 71%, 72%, 73%, 74%, 75%, or 80% by weight of a non-opioid analgesic. In some instances, the non-opioid analgesic is acetaminophen. In some instances, the pharmaceutical composition is a solid oral pharmaceutical composition.

In some cases, a first layer in a pharmaceutical composition described herein comprises between about 10% to about 90% by weight of microcrystalline cellulose. In some instances, the first layer comprises about 10%, 11%, 12%, 13%, 14%, 15%, 16%, 17%, 18%, 19%, 20%, 21%, 72%, 23%, 74%, 25%, 26%, 27%, 27.3%, 27.5%, 28%, 29%, 30%, 31%, 32%, 33%, 34%, 35%, 36%, 37%, 38%, 39%, 40%, 41%, 42%, 43%, 44%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% by weight of microcrystalline cellulose. In some instances, the pharmaceutical composition is a solid oral pharmaceutical composition.

In some cases, a first layer in a pharmaceutical composition described herein comprises between about 0.1% to about 10% by weight of hydroxypropyl methylcellulose. In some instances, the first layer comprises about 0.1%, 0.2%, 0.3%, 0.4%, 0.5%, 0.6%, 0.7%, 0.8%, 0.9%, 1%, 1.5%, 2%, 2.5%, 2.6%, 2.7%, 2.8%, 2.9%, 3%, 3.5%, 4%, 4.5%, 5%, 5.5%, 6%, 6.5%, 7%, 7.5%, 8%, 8.5%, 9%, 9.5%, or 10% by weight of hydroxypropyl methylcellulose. In some instances, the pharmaceutical composition is a solid oral pharmaceutical composition.

In some cases, a first layer in a pharmaceutical composition described herein comprises between about 0.05% to about 5% by weight of magnesium stearate. In some instances, the first layer comprises about 0.05%, 0.1%, 0.15% 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 4.65%, 2.7%, 2.75%, 2.8%, 2.85%, 2.9%, 2.95%, or 3%, 3.5%, 4%, 4.5%, or 5% by weight of magnesium stearate. In some instances, the pharmaceutical composition is a solid oral pharmaceutical composition.

In some cases, a first layer in a pharmaceutical composition described herein comprises between about 0.05% to about 5% by weight of stearic acid. In some instances, the first layer comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, or 3%, 3.5%, 4%, 4.5%, or 5% by weight of stearic acid. In some instances, the pharmaceutical composition is a solid oral pharmaceutical composition.

In some cases, a first layer in a pharmaceutical composition described herein comprises between about 0.05% to about 5% by weight of sodium starch glycolate. In some instances, the first layer comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95% or 3%, 3.5%, 4%, 4.5%, or 5% by weight of sodium starch glycolate. In some instances, the pharmaceutical composition is a solid oral pharmaceutical composition.

In some cases, a first layer in a pharmaceutical composition described herein comprises between about 0.0500 to about 5% by weight of croscarmellose sodium. In some instances, the first layer comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, or 3%, 3.5%, 4%, 4.5%, or 5%, by weight of croscarmellose sodium. In some instances, the pharmaceutical composition is a solid oral pharmaceutical composition.

In some cases, a first layer in a pharmaceutical composition described herein comprises between about 5% to about 50% by weight of lactose monohydrate. In some instances, the first layer comprises about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 27.3%, 27.5%, 28%, 29%, 30%, 31%, 32%, 33%, 34%, 35%, 36%, 37%, 38%, 39%, 40%, 41%, 42%, 43%, 44%, 45%, or 50% by weight of lactose monohydrate. In some instances, the pharmaceutical composition is a solid oral pharmaceutical composition.

In some cases, a first layer in a pharmaceutical composition described herein comprises between about 5% to about 50% by weight of mannitol. In some instances, the first layer comprises about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 27.3%, 27.5%, 28%, 29%, 30%, 31%, 32%, 33%, 34%, 35%, 36%, 37%, 38%, 39%, 40%, 41%, 42%, 43%, 44%, 45%, or 50% by weight of mannitol. In some instances, the pharmaceutical composition is a solid oral pharmaceutical composition.

In some cases, a first layer in a pharmaceutical composition described herein does not comprise croscarmellose sodium. In some instances, the first layer does not comprise sodium starch glycolate. In some instances, the first layer does not comprise lactose monohydrate. In some instances, the first layer does not comprise mannitol. In some instances, the pharmaceutical composition is a solid oral pharmaceutical composition.

In some cases, a pharmaceutical composition described herein comprises at least two layers. In some instances, the second layer comprises one or more excipients, including but not limited to microcrystalline cellulose, croscarmellose sodium, and magnesium stearate. In some instances, the microcrystalline cellulose is silicified microcrystalline cellulose. In some instances, the second layer further comprises an anti-emetic. In sonic instances, the anti-emetic is promethazine or a pharmaceutically acceptable salt thereof. In some instances, the anti-emetic is promethazine hydrochloride. In some instances, the pharmaceutical composition is a solid oral. pharmaceutical composition.

In some cases, a pharmaceutical composition described herein comprises at least two layers. In some instances, a second layer in a pharmaceutical composition described herein comprises about 5 mg to about 30 mg of an anti-emetic. In some instances, a second layer in a pharmaceutical composition described herein comprises about 1 mg to about 100 mg of an anti-emetic. In some instances, a second layer comprises about 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 42.5 mg. 45 mg. 47.5 mg, 50 mg, 55 mg, 60 mg, 65 mg, 70 mg, 75 mg, 80 mg, 85 mg, 90 mg, 95 mg, or 100 mg of an anti-emetic. In some instances, a second layer in a pharmaceutical composition described herein comprises about 5 mg to about 30 mg of promethazine or a pharmaceutically acceptable salt thereof. In some instances, a second layer in a pharmaceutical composition described herein comprises about 1 mg to about 100 mg of promethazine or a pharmaceutically acceptable salt thereof. In some instances, a second layer comprises about 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg. 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, or 100 mg of promethazine or a pharmaceutically acceptable salt thereof. in some instances, a second layer in a pharmaceutical composition described herein comprises about 5 mg to about 30 mg of promethazine hydrochloride. In some instances, a second layer in a pharmaceutical composition described herein comprises about 1 mg to about 100 mg of promethazine hydrochloride. In some instances, a second layer comprises about 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, or 100 mg of promethazine hydrochloride. In some instances, the pharmaceutical composition is a solid oral pharmaceutical composition.

In some cases, a pharmaceutical composition described herein comprises at least two layers. In some instances, a second layer in a pharmaceutical composition described herein comprises about 75 mg to about 250 mg of microcrystalline cellulose. In some instances, a second layer in a pharmaceutical composition described herein comprises about 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg 116 mg, 117 mg, 118 mg, 119 mg, 120 mg, 121 mg, 121.5 mg 122 mg, 12.3 mg, 12.4 mg, 12.5 mg, 12.6 mg, 12.7 mg, 12.8 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg,150 mg, 160 mg, 170 mg, 180 mg, 190 mg, 20( ) mg, 210 mg, 220 mg, 230 mg, 240 mg, or 250 mg of microcrystalline cellulose. In some instances, the pharmaceutical composition is a solid oral pharmaceutical composition.

In some cases, a pharmaceutical composition described herein comprises at least two layers. In some instances, a second layer in a pharmaceutical composition described herein comprises about 5 mg to about 30 mg of croscarmellose sodium. In some instances, a second layer in a pharmaceutical composition described herein comprises about 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12 mg, 12.1 mg, 12.2 mg, 12.3 mg, 12.4 mg, 12.5 mg, 12.6 mg 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 m , 1.5.2 mg, 15.3 mg, 15.4 mg, 11.5.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg, 20 mg, 21 mg, 22 mg, 23 mg, 24 mg, 25 mg, 26 mg, 27 mg, 28 mg, 29 mg, or 30 mg of croscarmellose sodium. In some instances, the pharmaceutical composition is a solid oral pharmaceutical composition.

In some cases, a pharmaceutical composition described herein comprises at least two layers. In some instances, a second layer in a pharmaceutical composition described herein comprises about 0.05 mg to about 5 mg of magnesium stearate. In some instances, a second layer in a pharmaceutical composition described herein comprises about 0.05 mg, 0.1 mg, 0.15 mg, 0.16 mg, 0.17 mg, 0.175 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 .8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg or 5 mg, of magnesium stearate. In some instances, the pharmaceutical composition is a solid oral pharmaceutical composition.

In some cases, a pharmaceutical composition described herein comprises at least two layers. In some instances, a second layer comprises from about 5 mg to about 30 mg of an anti-emetic, from about 75 mg to about 250 mg of microcrystalline cellulose, from about 5 mg to about 30 mg croscarmellose sodium, and from about 0.05 mg to about 5 mg magnesium stearate. 1111 some instances, the second layer comprises one or more additional pharmaceutically acceptable excipients, salts, and/or carriers disclosed herein. In some instances, a second layer comprises from about 5 mg to about 30 mg of promethazine or a pharmaceutically acceptable salt thereof, from about 75 mg to about 250 mg of microcrystalline cellulose, from about 5 mg to about 30 mg croscarmellose sodium, and from about 0.05 mg to about 5 mg magnesium stearate. 1111 some instances, a second layer comprises from about 5 mg to about 30 mg of promethazine hydrochloride, from about 75 mg to about 250 mg of microcrystalline cellulose, from about 5 mg to about 30 mg croscarmellose sodium, and from about 0.05 mg to about 5 mg magnesium stearate. In some instances, a second layer in a pharmaceutical composition described herein comprises about 12.5 mg of promethazine hydrochloride, about 101.5 mg of microcrystalline cellulose, about 15 mg of croscarmellose sodium, and about 1 mg of magnesium stearate. In some instances, a second layer in a pharmaceutical composition described herein comprises about 12.5 mg of promethazine hydrochloride, about 121.5 mg of microcrystalline cellulose, about 15 mg of croscarmellose sodium, and about 1 mg of magnesium stearate. In some instances, the pharmaceutical composition is a solid oral pharmaceutical composition.

In some cases, a pharmaceutical composition described herein comprises at least two layers. In some instances, a second layer in a pharmaceutical composition described herein comprises between about 1% to about 25% by weight of an antiemetic. In some instances, the second layer comprises about 1%, 2%, 3%, 4%, 5%, 6%, 7%, 8%, 8.3%, 8.5%, 9%, 10%, 11%, 12%, 13%, 14%, 15%, 16%, 17%, 18%, 19%, 20%, 21%, 22%, 73%, 24%, or 25% by weight of an antiemetic. In some instances, a second layer in a pharmaceutical composition described herein comprises between about 1% to about 25% by weight of promethazine or a pharmaceutically acceptable salt thereof. In some instances, the second layer comprises about 1%, 2%, 3%, 4%, 5%, 6%, 7%, 8%, 8.3%, 8.5%, 9%, 10%, 11%, 12%, 13%, 14%, 15%, 16%, 17%, 18%, 19%, 20%, 21%, 22%, 23%, 24%, or 25% by weight of promethazine or a pharmaceutically acceptable salt thereof. In some instances, a second layer in a pharmaceutical composition described herein comprises between about 1% to about 25% by weight of promethazine hydrochloride. In some instances, the second layer comprises about 1%, 2%, 3%, 4%, 5%, 6%, 7%, 8%, 8.3%, 8.5%, 9%, 10%, 11%, 12%, 13%, 14%, 15%, 16%, 17%, 18%, 19%, 20%, 21%, 22%, 23%, 24%, or 25% by weight of promethazine hydrochloride. In some instances, the pharmaceutical composition is a solid oral pharmaceutical composition.

In some cases, a pharmaceutical composition described herein comprises at least two layers. In some instances, a second layer in a pharmaceutical composition described herein comprises between about 65% to about 95% by weight of microcrystalline cellulose. In some instances, the second layer comprises about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% by weight of microcrystalline cellulose. In some instances, the pharmaceutical composition is a solid oral pharmaceutical composition.

In some cases, a pharmaceutical composition described herein comprises at least two layers. In some instances, a second layer in a pharmaceutical composition described herein comprises between about 5% to about 20% by weight of croscarmel lose sodium. In some instances, the second layer comprises about 5%, 6%, 7%, 8%, 9%, 10%, 11%, 12%, 13%, 14%, 15%, 16%, 17%, 18%, 19%, or 20% by weight of croscartnellose sodium. In some instances, the pharmaceutical composition is a solid oral pharmaceutical composition.

In some cases, a pharmaceutical composition described herein comprises at least two layers. In some instances, a second layer in a pharmaceutical composition described herein comprises between about 0.05% to about 5% by weight of magnesium stearate. In some instances, the second layer comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.5%, 4%, 4.5%, or 5% by weight of magnesium stearate. In some instances, the pharmaceutical composition is a solid oral pharmaceutical composition.

### In Vitro Dissolution Methods

In vitro dissolution studies are performed in accordance with guidelines from the United States Pharmacopeial Convention, the European Pharmacopoeia, and/or the Japanese Pharmacopoeia. In some instances, the pharmaceutical compositions described herein are tested for in vitro dissolution. In some instances, tablets are tested for in vitro dissolution. In some instances, more than one of the same tablets can be tested for in vitro dissolution. In some instances, the more than one of the same tablets is called a test batch. In some instances, the size of the test batch is at least about 10% of the largest batch planned. In some instances, the size of the test batch is 100,000 tablets. In some instances, the size of the test batch is less than 100,000 tablets. The size of the test batch can be more than 100,000 tablets.

In some cases, dissolution apparatus is a USP Rotating Paddle Apparatus 2 with an automated sampling station (e.g., VK-8000 or equivalent). In some instances, dissolution fluid is de-aerated 0.01 N HCl, maintained at 37.0+/−0.5° C. during dissolution procedure. In some instances, the fluid is prepared by diluting concentrated HCl in de-aerated water, and mixed. To measure peaks, a dual wavelength detector (e.g., Hitachi L-2420) is used, or in some instances, two separate chromatographic systems are used in order to measure the peaks at two different wavelengths.

In some cases, standard solution preparation is made as follows: each ingredient is weighed in a volumetric flask, and diluted to volume with dissolution media. The resulting solution is mixed to form a stock solution. Each aliquot of stock standard solutions is diluted with dissolution fluid and mixed to produce a final standard solution.

In some instances, dissolution test solutions are prepared in 0.01 N HCl using the USP Rotating Paddle Apparatus at 50 WM. An aliquot of the dissolution solution is filtered and an aliquot is chromatographed on a 50-mm×4.6-mm (i.d.) Waters sunFire™ C₁₈, 3.5-μm particle size column using a gradient HPLC method. Mobile phase A consists of water/acetonitrile/TFA, 950/50/2 (v/v/v) and mobile phase B consisted of water/acetonitrile/TFA, 50/950/1.5 (v/v/v). The flow rate can be 2.0 mL/minute. The amount of oxycodone release is determined at 230 nm by comparing the area obtained for the peak due to oxycodone in the chromatogram of the dissolution test solution to that obtained for the corresponding peak in a chromatogram of a standard solution. The amount of promethazine released is determined at 230 nm by comparing the area obtained for the peak due to promethazine in the chromatogram of the dissolution test solution to that obtained for the corresponding peak in a chromatogram of a standard solution.

In some instances, paddle speed is 50 rpm; pull volume is 10 mL (no replacement); Pull points: 5, 10, 15, 20, 25, 30, 45 and 60 minutes. The amount of each component dissolved in the dissolution medium is determined by HPLC. In some instances, the method uses a high purity, bonded C18 stationary phase and a binary mobile phase consisting of an appropriate buffer and organic modifier.

In some instances, dissolution fluid is preheated to 37° C. and placed into each vessel. Tablets are weighed and placed in vessels respectively. At prescribed time intervals, an aliquot of the dissolution fluid is drawn using the automated sampling station equipped with a 35 μm full flow filter connected to a sampling probe. Filtrate is allowed to cool to room temperature, to produce a final sample solution. Fluid withdrawn is not replaced. Samples are injected in HPLC for analysis after a baseline is established. The resolution between each peak is calculated, as well as the tailing factor. In some instances, the five replicate injections are not more than 2.0% RSD. In some instances, 50 μL aliquots of standard and sample solutions are subjected to liquid chromatography.

In some instances, a 50-μL aliquot is chromatographed on a 150-mm×4.6-mm (i.d.) Phenomenex Kinetex™ C₁₈, 2.6-μm particle size column using a gradient HPLC method. Mobile phase A consisted of water/KH₂PO₄ (monohydrate)lhepatanesulfonic sodium, 1000 g/2 g/1 g, pH 3.0±0.05, and mobile phase B consisted of 100% acetonitrile. The flow rate is 1.0 mL/minute. For example, the amount of oxycodone released is determined at 280 nm by comparing the area obtained for the peak due to oxycodone in the chromatogram of the dissolution test solution to that obtained for the corresponding peak in a chromatogram of a standard solution. The amount of promethazine released is determined at 280 nm by comparing the area obtained for the peak due to promethazine the chromatogram of the dissolution test solution to that obtained for the corresponding peak in a chromatogram of a standard solution.

In some instances, USP Apparatus 1 Basket speed is 50 rpm; pull volume is 1.5 ml using an auto sampler; pull points: 5, 10, 15, 30, and 60 minutes, and 90 minutes spin out. The amount of each component dissolved in the dissolution medium is determined by HPLC. In some instances, the method uses a high purity, bonded C18 stationary phase and a binary mobile phase consisting of an appropriate buffer and organic modifier.

In some instances, the amount released per tablet is determined using Equation

$$\frac{mg Released}{Tablet} = \frac{Au}{As} \times Cs \times Vn$$

where:
Au=The peak area response obtained in the chromatogram of the dissolution test solution.
As=The peak area response obtained in the chromatogram of the standard solution.
Cs=The concentration of the standard solution.
Vn=The volume, in mL, of the dissolution solution at sampling time period n. It is calculated as follows:

Vn=[900−5(n−1)]

In some instances, the amount released as a percent of the label claim is determined using the following equation:

$$\% Released = \frac{mg \frac{Released}{Tablet}}{Label Claim} \times 100 \%$$

In some instances, the amount released at second and subsequent time periods in mg/tablet is determined using the following equation:

$$Wn = Un + 5 {\sum }_{i = 1}^{n = 1} \frac{Ui}{Vi}$$

where:
Wn=The mg released/tablet at time period n (corrected).
Un=The mg released/tablet at time n (uncorrected).
n=The current time period.
i=The time period index.
Ui=The mg released/tablet at time period i (uncorrected).
Vi=The volume, in mL, of the dissolution solution at sampling time period i. It is calculated as follows:

Vi=[900−5(i−1)]

In some instances, the released amount i calculated as follows:

$$\% Released = [ ( \frac{{R}_{u}}{{R}_{s}} \times {C}_{STD} \times {V}_{d} ) + {\sum }_{i = 1}^{n - 1} ( \frac{{R}_{i}}{{R}_{s}} \times {C}_{STD} \times {V}_{i} ) ] \times ( \frac{1}{LC} ) \times 100$$

Where:
   R_{U}=Area of Promethazine and Oxycodone in the Sample Solution
   R_{S}=Area of Promethazine and Oxycodone in all Standards
   C_{Std}=Concentration of Promethazine and Oxycodone in the Standard preparation (mg/mL)
   V_{d}=Volume of dissolution medium at the pull time (mL)
   Rᵢ=Peak area of Promethazine and Oxycodone obtained from the sample preparation at the individual pull points
   Vᵢ=Volume of the sample removed from the vessel at the previous pull point (mL)
   LC=Label Claim (trig)

In some instances, in vitro dissolution testing is collected from at least about 24 tablets. In some instances, in vitro dissolution testing can be collected from at least about: 10, 12, 16, 18, 20, 22, 24, 26, 28, 30, 32, 34, 36, 40, 50, or 100 tablets. In some instances, in vitro dissolution testing can be collected from more than about: 10, 12, 16, 18, 20, 22, 24, 26, 28, 30, 32, 34, 36, 40, 50, 100 tablets or more. In some instances, in vitro dissolution testing can be collected from less than about: 10, 12, 16, 18, 20. 22, 24, 26. 28, 30, 32, 34, 36, 40, 50, 100 tablets or less. Tablets can be from the same test batch. In some instances, in vitro dissolution testing can be done on equal numbers of test and reference tablets (e.g. 12 test tablets and 12 reference tablets

Tablets can be added to a liquid, such as a dissolution medium, to initiate dissolution of the tablet and to permit sampling of the liquid to measure the amount of composition released from the tablet at one or more specific time points. Dissolution medium can be a solution. Dissolution medium can be a buffered solution. The buffered solution can have a pH of between about 4 and about 8. Dissolution medium can be an acid. The dissolution medium can be an acid between about 0.001N to about 0.1N, for example 0.1N hydrochloric acid. Dissolution medium can be dea.erated. Dissolution medium can contain dissolved gases. Dissolution medium can be about pH 7.4. Dissolution medium can be about pH 7.0. Dissolution medium can be about pH 6.0. Dissolution medium can be 0.05 M pH 7.4 phosphate buffer. Dissolution medium can be deaerated water. Dissolution medium can be an acid. Dissolution medium can be a base. Dissolution medium can be 0.1 N hydrochloric acid. Dissolution medium can be water. Dissolution medium can be pH 6.0 phosphate buffer solution. Solutes such as surfactants can be added to the dissolution medium.

In vitro dissolution testing is performed using an apparatus as described in the General Chapters and General Methods of the Harmonization section of the United States Pharmacopeia and the National Formulary (USP-NF), such as the General Methods 711 Dissolution Standards section. In some instances, a specific volume of dissolution medium can be placed into the vessel of the apparatus. A volume of about 500 mL dissolution medium can be added. A volume of about 900 mL dissolution medium can be added. A volume of about 1000 mL dissolution medium can be added. A volume of about: 200, 300, 400, 500, 600, 700, 750, 800, 900, 1000, 1100, 1200, 1300, 1400, 1500, 2000 mL dissolution medium can be added. In some instances, the volume of dissolution medium can not be less than 3 times that need to form a saturated solution of the composition. In some instances, the apparatus is a basket, for example, as described in the General Methods 711 Dissolution Standards of the USP-NF. In some instances, the basket apparatus is used for dissolution studies of capsules. In some instances, the basket apparatus is used for dissolution studies of controlled-release formulations. In some instances, the apparatus is a paddle, for example, as described in the General Methods 711 Dissolution Standards of the USP-NF. In some instances, the paddle apparatus is used for dissolution studies of solid formulations. In some instances, the paddle apparatus is used for dissolution studies of tablets. In some instances, the apparatus is a reciprocating cylinder, for example, as described in the General Methods 711 Dissolution Standards of the USP-NF. In some instances, the reciprocating cylinder apparatus is used for dissolution studies of bead-type controlled-release dosage forms. In some instances, the apparatus is a flow cell, for example, as described in the General Methods 711 Dissolution Standards of the USP-NF. In some instances, the flow cell apparatus is used for dissolution studies of controlled-release dosage forms. In some instances, the flow cell apparatus is used for dissolution studies of formulations with poor solubility. In some instances, the apparatus is a paddle over disk. In some instances, the paddle over disk apparatus is used for dissolution studies of transdermal dosage forms. in some instances, the apparatus is a cylinder. In some instances, the cylinder apparatus is used for dissolution studies of transdermal dosage forms. In some instances, the apparatus is a reciprocating disk. In some instances, the reciprocating disk apparatus is used for dissolution studies of non-disintegrating oral controlled-release dosage forms. In some instances, a size 40-mesh screen is used in the apparatus. In some instances, the apparatus is calibrated with the USP Salicyclic Acid and Prednisone Calibrator Tablets.

In some cases, a dissolution apparatus disclosed herein is assembled. In some instances, the dissolution medium is equilibrated to about 35+/−0.5 degrees Fahrenheit. In some instances, the dissolution medium is equilibrated to about internal body temperature. In some instances, one test tablet or one reference tablet is placed into the apparatus in the dissolution medium. In some instances, the apparatus is immediately set to operate. In some instances, the apparatus is set to operate. In some instances, the apparatus is set to operate at about 25 rotations per minute (rpm). In some instances, the apparatus is set to operate at about 50 rpm. In some instances, the apparatus is set to operate at about 100 rpm. In some instances, the apparatus is set to operate at about: 10, 20, 25, 30, 35, 40, 45, 50, 55, 60, 65, 70, 75, 80, 85, 90, 95, 100, 105, 110, 115, 120, 125, 130, 135, 140, 145, 150, 200, or 300 rpm. In some instances, samples of dissolution medium are withdrawn.

In some cases, one or more samples of dissolution medium are withdrawn at one or more specified times, in some instances, samples are withdrawn following the addition of the tablet into the dissolution medium in the vessel of the apparatus. In some instances, one or more samples are collected at one or more times. In some instances, one or more samples are collected at three different times. In some instances, the final time is chosen to show complete release of the composition. In some instances, one or more samples are collected at about: 5, 10, 15, 20, 30, 45, 60, 90, or 180 minutes. In some instances, a volume of dissolution medium is withdrawn from the vessel. In some instances, the volume is defined. In some instances, the volume is unknown, in some instances, the volume of dissolution medium withdrawn is the same for all times in a single in vitro dissolution study. In some instances, the volume of dissolution medium is withdrawn from the zone midway between the surface of the dissolution medium and the top of the rotating basket or blade and not less than 1 cm from the vessel wall. In some instances, the volume of dissolution medium withdrawn from the vessel is replaced with an equal volume of fresh dissolution medium. In some instances, the volume of dissolution medium withdrawn from the vessel is not replaced. In this instance, the volume change is incorporated into the dissolution calculation. In some instances, the temperature of the dissolution medium in the vessels is verified at one or more time points. In some instances, in vitro dissolution testing is repeated with one or more additional tablets. In some instances, withdraw of dissolution medium from the vessel is automated.

In some cases, a bilayer tablet provides an effective amount of two pharmaceutically active agents for about 4-6 hours following oral administration. In some instances, the bilayer tablet provides an effective amount of two active agents for about 12 hours, about 24 hours or about 48 hours following administration. In some instances, the two pharmaceutically active agents are provided in 4-6 hour, 12 hour, 24 hour or 48 hour dosing intervals. In some instances, the bilayer tablet is capable of providing two pharmaceutically active agents disclosed herein in the foregoing dosing intervals.

In some cases, pharmaceutical compositions described herein comprise a first layer. In some instances, the first layer comprises an opioid analgesic. In some instances, the second layer is an immediate-release layer. In some instances, the first layer is a controlled-release layer. In some instances, the second layer has a faster dissolution rate than the first layer. In some instances, the antiemetic is released before the opioid analgesic. In some instances, the release is complete release. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the opioid analgesic is released within the first 5 minutes following contact with a dissolution fluid. In some instances, at least about 62% of the opioid analgesic is released within the first 5 minutes. In some instances, at least about 78% of the opioid analgesic is released within the first 5 minutes. In some instances, at least about 82% of the opioid analgesic is released within the first 5 minutes. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the opioid analgesic is released within the first 10 minutes following contact with a dissolution fluid. In some instances, at least about 72% of the opioid analgesic is released within the first 10 minutes. in some instances, at least about 84% of the opioid analgesic is released within the first 10 minutes. In some instances, at least about 86% of the opioid analgesic is released within the first 10 minutes. In some instances, at least about 50%, about 51%, about 52%, about 53%, about 54%, about 55′%©, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the opioid analgesic is released within the first 15 minutes following contact with a dissolution fluid. In some instances, at least about 76% of the opioid analgesic is released within the first 15 minutes. In some instances, at least about 87% of the opioid analgesic is released within the first 15 minutes. In some instances, at least about 88% of the opioid analgesic is released within the first 15 minutes. In some instances, at least about 50%, about 51%, about 52%, about 53%, about 54%, about 55%, about 60%. about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%. about 93%, about 94%, about 95%, about 96%, about 97%, about 98%, about 99%, about 100%, or more of the opioid analgesic is released within the first 30 minutes following contact with a dissolution fluid. In some instances, at least about 83% of the opioid analgesic is released within the first 30 minutes. In some instances, at least about 92% of the opioid analgesic is released within the first 30 minutes. In some instances, at least about 95% of the opioid analgesic is released within the first 30 minutes. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the opioid analgesic is released within the first 45 minutes following contact with a dissolution fluid. In some instances, at least about 93% of the opioid analgesic is released within the first 45 minutes. In some instances, at least about 95% of the opioid analgesic is released within the first 45 minutes. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the opioid analgesic is released within the first 60 minutes following contact with a dissolution fluid. In some instances, at least about 91% of the opioid analgesic is released within the first 60 minutes. In some instances, at least about 95% of the opioid analgesic is released within the first 60 minutes. In some instances, at least about 97% of the opioid analgesic is released within the first 60 minutes. In some instances, the opioid analgesic is oxycodone or a pharmaceutically acceptable salt thereof. In some instances, the oxycodone or a pharmaceutically acceptable salt thereof is oxycodone hydrochloride. In some instances, the opioid analgesic is hydrocodone or a pharmaceutically acceptable salt thereof. In some instances, the hydrocodone or a pharmaceutically acceptable salt thereof is hydrocodone bitartrate. In some instances, the pharmaceutical composition is a solid oral pharmaceutical composition.

In some cases, pharmaceutical compositions described herein comprise a second layer. In some instances, the second layer comprises an anti-emetic. In some instances, the second layer is an immediate-release layer. In some instances, the first layer is a controlled-release layer. In some instances, the second layer has a faster dissolution rate than the first layer. In some instances, the antiemetic is released before the opioid analgesic. In some instances, the release is complete release. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95 about 96%, about 97%, about 98%, about 99%, about 100%, or more of the anti-emetic is released within the first 5 minutes following contact with a dissolution fluid. In some instances, at least about 77% of the anti-emetic is released within the first 5 minutes. In some instances, at least about 81% of the anti-emetic is released within the first 5 minutes, in some instances, at least about 89% of the anti-emetic is released within the first 5 minutes. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the antiemetic is released within the first 10 minutes. In some instances, at least about 78% of the antiemetic is released within the first 10 minutes. In some instances, at least about 85% of the antiemetic is released within the first 10 minutes. In some instances, at least about 93% of the antiemetic is released within the first 10 minutes. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the anti-emetic is released within the first 15 minutes. In some instances, at least about 81% of the antiemetic is released within the first 15 minutes. In some instances, at least about 86% of the antiemetic is released within the first 15 minutes. in some instances, at least about 95% of the antiemetic is released within the first 15 minutes. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the anti-emetic is released within the first 30 minutes. In some instances, at least about 87% of the anti-emetic is released within the first 30 minutes. In some instances, at least about 88% of the anti-emetic is released within the first 30 minutes. In some instances, at least about 99% of the anti-emetic is released within the first 30 minutes. In some instances, at least about 50%, about 51%, about 52%, about 53%, about 54%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the anti-emetic is released within the first 45 minutes. In some instances, at least about 85% of the anti-emetic is released within the first 45 minutes. In some instances, at least about 95% of the anti-emetic is released within the first 45 minutes. In some instances, at least about 97% of the anti-emetic is released within the first 45 minutes. In some instances, at least about 50%, about 51%, about 52%, about 53%, about 54%, about 5.5%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or more of the anti-emetic is released within the first 60 minutes. In some instances, at least about 87% of the anti-emetic is released within the first 60 minutes. In some instances, at least about 88% of the anti-emetic is released within the first 60 minutes. In some instances, at least about 100% of the anti-emetic is released within the first 60 minutes. In some instances, the antiemetic is promethazine or a pharmaceutically acceptable salt thereof. In some instances, the promethazine or a pharmaceutically acceptable salt thereof is promethazine hydrochloride. In some instances, the pharmaceutical composition is a solid oral pharmaceutical composition.

In some cases, the second layer is an immediate-release layer. In some instances, the first layer is a controlled-release layer. In some instances, the second layer has a faster dissolution rate than the first layer. In some instances, the antiemetic is released before the opioid analgesic. In some instances, the release is complete release. In some instances, a coated tablet has the same or similar dissolution rate as an equivalent uncoated tablet. In some instances, the coated tablet characteristics (formulation, size, weight, weight ratio, coating thickness) are determined based on the coating characteristic such that dissolution rate of the tablet core is not significantly affected by the coating. For example, the tablet geometry or tablet formulation can be modified to match a surface are requirement determined to be necessary to achieve a desired dissolution rate. In other examples, the polyethylene oxide molecular weight or concentration can be adjusted to allow a more rapid hydration of the polymer and dispersion of the coating.

In some cases, a solid oral pharmaceutical composition disclosed herein, following contact with a dissolution fluid as measured by USP Apparatus 2 (Paddle), has one or more of the following release rates: about 40-60% of the oxycodone or the pharmaceutically acceptable salt thereof is released within about 5 minutes; about 50-80% of the oxycodone or the pharmaceutically acceptable salt thereof is released within about 10 minutes; about 60-80% of the oxycodone or the pharmaceutically acceptable salt thereof is released within about 15 minutes; about 70-90% of the oxycodone or the pharmaceutically acceptable salt thereof is released within about 30 minutes; or about 70-100% of the oxycodone or the pharmaceutically acceptable salt thereof is released within about 60 minutes. In some instances, the solid oral pharmaceutical composition has one or more of the following release rates: about 51% of the oxycodone or the pharmaceutically acceptable salt thereof is released within about 5 minutes; about 64% of the oxycodone or the pharmaceutically acceptable salt thereof is released within about 10 minutes; about 69% of the oxycodone or the pharmaceutically acceptable salt thereof is released within about 15 minutes; about 77% of the oxycodone or the pharmaceutically acceptable salt thereof is released within about 30 minutes; or about 83% of the oxycodone or the pharmaceutically acceptable salt thereof is released within about 60 minutes.

In some cases, a solid oral pharmaceutical composition disclosed herein, following contact with a dissolution fluid as measured by USP Apparatus 2 (Paddle), has one or more of the following release rates: about 80-100% of the anti emetic is released within about 5 minutes; about 85-100% of the antiemetic is released within about 10 minutes; about 90-100% of the antiemetic is released within about 15 minutes; about 95-100% of the antiemetic is released within about 30 minutes; or about 98-100% of the antiemetic is released within about 60 minutes. In some instances, the solid oral pharmaceutical composition has one or more of the following release rates: about 95% of the antiemetic is released within about 5 minutes; about 97% of the antiemetic is released within about 10 minutes; about 98% of the antiemetic is released within about 15 minutes; about 99% of the antiemetic is released within about 30 minutes; or about 100% of the antiemetic is released within about 60 minutes.

### Dosage Forms

In some cases, a pharmaceutical composition described herein can be formulated as a dosage form. In some instances, a dosage form can be an oral dosage form such as a tablet, capsule, cachet, soft gelatin capsule, hard gelatin capsule, extended release capsule, tannate tablet, oral disintegrating tablet, multi-layer tablet, effervescent tablet, bead, powder, granule, liquid, oral suspension, chewable lozenge, oral solution, lozenge, lollipop, oral syrup, sterile packaged powder including pharmaceutically-acceptable excipients, other oral dosage forms, or any combination thereof. In some instances, the pharmaceutical composition is a liquid composition. In some instances, a pharmaceutical composition is a solid composition. In some instances, a pharmaceutical composition is a solid oral composition. In some instances, a solid oral composition can be a tablet. A tablet can be formed by compression molding. Such compressed tablets can be prepared by compressing in a suitable machine the three or more agents and a pharmaceutically acceptable carrier. in some instances, solid dosage forms, such as tablets, beads, or granules, can be coated, such as with a sugar coating. In some instances, the pharmaceutical composition is a solid oral pharmaceutical composition.

In some cases, described herein are multi-layer tablets. In some instances, the tablet comprises at least two layers. In some instances, the tablet is a bi-layer tablet. In some instances, the bi-layer tablet comprises a first layer and a second layer. In some instances, the tablet is rectangular, tubular, oblong, circular, oval or in a capsule form.

In some cases, described herein are tablets comprising an inner core and an outer layer or coating layer. In some instances, the inner core comprises multiple matrices. In some instances, the inner core comprises two matrices. In some instances, the inner core comprises a first matrix and a second matrix, wherein the first matrix comprises an opioid analgesic, and wherein the second matrix comprises an antiemetic. In some instances, the outer layer comprises an abuse deterrent agent. In some instances, the abuse deterrent agent is a gelling agent. In some instances, the gelling agent is a polymer. In some instances, the gelling agent is polyalkylene oxide. In some instances, the gelling agent is a polyethylene oxide.

In some cases, described herein are tablets comprising an inner core and an outer layer or coating layer. In some instances, the inner core comprises multiple layers. In some instances, the inner core comprises two layers. In some instances, the inner core comprises a first layer and a second layer, wherein the first layer comprises an opioid analgesic, and wherein the second layer comprises an antiemetic. In some instances, the outer layer comprises an abuse deterrent agent. In some instances, the abuse deterrent agent is a gelling agent. In some instances, the gelling agent is a polymer. In some instances, the gelling agent is polyalkylene oxide. In some instances, the gelling agent is a polyethylene oxide.

In some cases, described herein are tablets comprising an inner core and an outer layer or coating layer. In some instances, the inner core comprises multiple layers. In some instances, the inner core comprises two layers. In some instances, the inner core comprises a first layer and a second layer, wherein the first layer comprises an opioid analgesic and a non-opioid analgesic, and wherein the second layer comprises an antiemetic. In some instances, the outer layer comprises an abuse deterrent agent. In some instances, the abuse deterrent agent is a gelling agent. In some instances, the gelling agent is a polymer. In some instances, the gelling agent is polyalkylene oxide. In some instances, the gelling agent is a polyethylene oxide.

In some cases, described herein are tablets comprising an inner core and an outer layer or coating layer. In some instances, the inner core comprises a first layer and a second layer. In some instances, the total layer weight of the first layer is from about 20 to about 750 mg, such as about 35 mg, about 110 mg, about 12.0 mg, about 130 mg, about 140 mg, about 150 trig, about 160 mg, about 170 mg, about 180 mg, about 190 mg , about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 510 mg, about 520 mg, about 530 mg, about 540 mg, about 55 mg, about 560 mg, about 570 mg, about 580 mg, about 590 mg, about 600 mg, or about 650 mg. In some instances, the total layer weight of the first layer is about 35 mg. In some instances, the total layer weight of the first layer is about 200 mg. In some instances, the total layer weight of the first layer is about 550 mg.

In some cases, described herein are tablets comprising an inner core and an outer layer or coating layer. In some instances, the inner core comprises a first layer and a second layer. In some instances, the total layer weight of the second layer is from about 20 to about 250 mg, such as about 20 mg, about 25 mg, about 30 mg, about 35 mg, about 40 mg, about 45 mg, about 50 mg, about 55 mg, about 60 mg , about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 18( )mg, about 190 mg, about 200 mg, about 210 mg, about 220 mg, about 230 mg, about 240 mg, or about 250 mg. In some instances, the total layer weight of the second layer is about 130 mg. in some instances, the total layer weight of the second layer is about 150 mg.

In some cases, a pharmaceutical composition described herein comprises two layers, wherein the first layer comprises an opioid analgesic and the second layer comprises an antiemetic and wherein a ratio by weight of the first layer to the second layer is between about 1:0.1 to about 1:5. In some instances, the ratio by weight of the first layer to the second layer is about 1:0.1, about 1:0.2, about 1:0.25, about 1:0.3, about 1:0.4, about 1:0.5, about 1:0.6, about 1:0.7, about 1:0.8, about 1:0.9, about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.1, about 1:2.2, about 1:2.3, about 1:2.4, about 1:2.5, about 1:2.6, about 1:2.7, about 1:2.8, about 1:2.9, about 1:3, about 1:3.1, about 1:3.2, about 1:3.3, about 1:3.4, about 1:3.5, about 1:3.6, about 1:3.7, about 1:3.8, about 1:3.9, about 1:4, about 1:4.1, about 1:4.2, about 1:4.3, about 1:4.4, about 1:4.5, about 1:4.6, about 1:4.7, about 1:4.8, about 1:4.9, or about 1:5. In some instances, the ratio by weight of the first layer to the second layer is about 1:0.25. In some instances, the ratio by weight of the first layer to the second layer is between about 1:3 to about 1:4. In some instances, the ratio by weight of the first layer to the second layer is about 1:3.5. In some instances, the ratio by weight of the first layer to the second layer is about 1:3.6. In some instances, the ratio by weight of the first layer to the second layer is about 1:3.7. In some instances, the pharmaceutical composition is a solid oral pharmaceutical composition.

In some cases, a pharmaceutical composition described herein is in a form that achieves a hardness of from about 5 to about 15 kiloponds, including the hardness of any single number within this range, and has a thickness from about 5 to about 10 min, including the thickness of any single number within this range. in some instances, the tablet has a hardness of about 9.5 kiloponds or about 12.5 kiloponds. In some instances, the tablet has a hardness of about 5 kiloponds, about 5.5 kiloponds, about 6 kiloponds, about 6.5 kiloponds, about 7 kiloponds, about 7.5 kiloponds, about 8 kiloponds, about 8.5 kiloponds, about 9 kiloponds, about 9.5 kiloponds, about 10 kiloponds, about 10.5 kiloponds, about 11 kiloponds, about 11.5 kiloponds, about 12 kiloponds, about 12.5 kiloponds, about 13 kiloponds, about 13.5 kiloponds, about 14 kiloponds, about 14.5 kiloponds, or about 15 kiloponds. In some instances, the tablet has a thickness of about 5 mm, about 5.5 mm, about 6 mm, about 6.5 mm, about 7 mm, about 7.5 mm, about 8 mm, about 8.5 mm, about 9 mm, about 9.5 mm, or about 10 mm. In some instances, the pharmaceutical composition is a solid oral pharmaceutical composition.

In some cases, a pharmaceutical composition described herein is administered to a subject at about every 4 to about 6 hours or about every 8 hours or less often. In some instances, the pharmaceutical compositions are administered once daily. In some instances, the pharmaceutical compositions are administered twice daily in some instances, the pharmaceutical compositions are administered three, four, five or six times in a single a day. In some instances, the pharmaceutical composition is a solid oral pharmaceutical composition.

In some cases, a pharmaceutical composition is a solid oral pharmaceutical composition. In some instances, the solid oral pharmaceutical composition described herein is rectangular, tubular, oblong, circular, round, oval in shape, or in a capsule form. In some instances, the solid oral pharmaceutical composition is a tablet. In some instances, the tablet is rectangular, tubular, oblong, circular, round, oval in shape, or in a capsule form. In some instances, the tablet is round. In some instances, the tablet is oval. In some instances, the table is oblong.

In some instances, the solid oral pharmaceutical composition is round. In some examples, the diameter of the solid oral pharmaceutical composition is between about 0.1 mm and about 13 mm. For example, the diameter can be about 0.1 mm, 0.5 mm, 1 mm, 1.5 mm, 2 mm, 2.5 mm, 3 mm, 3.5 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 min, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.525 mm, 9.6 mm, 9.7 mm, 9.8 mm, 9.9 mm, 10 mm, 10.1 mm, 10.2 mm, 10.3 mm, 10.4 mm, 10.5 mm, 10.6 mm, 10.7 mm, 10.8 mm, 10.9 mm, 11 mm, 11.1 mm, 11.2 mm, 11.3 mm, 11.4 mm, 11.5 mm, 11.6 mm, 11.7 min, 11.8 mm, 1.9 mm, 12 mm, 12.1 mm, 12.2 mm, 12.3 mm, 12.4 mm, 12.5 mm, 12.6 mm, 12.7 mm, 12.8 mm, 12.9 min, or 13 mm. In some examples, the diameter is 9.525 mm. In some examples, the diameter is less than or equal to 5.49 mm, between 5.5 mm and 6.05 mm, between 6.06 mm and 6.73 mm, between 6.74 mm and 7.56 mm, between 7.57 mm and 9.19 mm, between 9.20 mm and 11.19 mm, or greater than 11.19 mm. In some examples, the diameter is less than 5.74 mm. In some examples, the diameter is less than 6.30 mm. In some examples, the diameter is less than 6.98 mm. In some examples, the diameter is less than 7.81 mm. In some examples, the diameter is less than 9.39 mm. In some examples, the diameter is less than 11.39 mm. In any of these examples, the diameter can be +/−0.051 mm. In some instances, the solid oral pharmaceutical composition is a tablet.

In some instances, the solid oral pharmaceutical composition is oval or oblong. In some examples, the height of the solid oral pharmaceutical composition is between about 0.1 mm and about 21 mm, .For example, the height of the tablet can be about 0.1 mm, 0.5 mm, 1 mm, 1.5 mm, 2 min, 2.5 mm, 3 mm, 3.5 min, 4 mm, 4.1 mm, 4.2 mm, 4.3 mm, 4.4 mm, 4.5 mm, 4.6 mm, 4.7 mm, 4.8 mm. 4.9 mm, 5 mm, 5.1 mm. 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 min, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 min, 8 mm, 8.1 mm, 8.2 trim, 8.3 mm, 8.4 min, 8.5 mm, 8.6 mm, 8.7 trim, 8.8 min, 8.9 min, 9 mm, 9.1 mm, 9.2 mm, 9.3 mm, 9.4 mm, 9.5 mm, 9.6 mm, 9.7 mm, 9.8 mm, 9.9 mm, 10 mm, 10.1 mm, 10.2 mm. 10.3 mm, 10.4 mm, 10.5 mm, 10.6 mm, 10.7 mm, 10.8 mm, 10.9 mm, 11 mm, 11.1 mm, 11.2 mm, 11.3 mm, 11.4 mm, 11.5 mm, 11.6 mm, 11.7 mm, 11.8 mm, 1.9 mm, 12 mm, 12.1 mm, 12.2 min, 12.3 mm, 12.4 mm, 12.5 min, 12.6 min, 12.7 mm, 12.8 trim, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm. 14.3 mm, 14.4 mm, 14.5 mm. 14.6 mm, 14.7 mm, 14.8 mm, 14.9 mm, 15 mm, 15.1 mm, 15.2 mm, 15.3 mm, 15.4 mm, 15.5 mm, 15.6 mm, 15.7 mm, 15.8 mm, 15.9 mm, 16 mm, 1.6.1 mm, 16.2 mm, 16.3 mm, 1.6A mm, 16.5 mm, 16.6 mm, 16.7 mm, 16.8 mm, 16.9 mm, 17 mm, 17.1 mm, 17.2. mm, 17.3 mm, 17.4 mm, 17.5 mm, 17.6 mm, 17.7 mm, 17.8 mm, 17.9 mm, 18 mm, 18.1 mm, 18.2 mm, 18.3 mm, 18.4 mm, 18.5 mm, 18.6 mm, 18.7 mm, 18.8 mm, 18.9 mm, 19 mm, 19.1 min, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm. or 21 mm. In some examples, the height is less than or equal to 8.74 mm, between 8.75 mm and 9.64 mm, between 9.65 mm and 10.34 mm, between 10.35 mm and 1.0.54 mm, between 10.55 mm and 13.06 mm, between 13.07 mm and 14.44 mm, between 11.4.45 mm and 15.65 mm, between 15.66 mm and 18.39 mm, or greater than 18.39 mm. In some examples, the height is less than 9.40 mm. In some examples, the height is less than 10.30 mm. In some examples, the height is less than 11.00 mm. In some examples, the height is less than 11.20 mm. In Sone examples, the height is less than 15.10 mm. In Sone examples, the height is less than 13.72 mm. In some examples, the height is less than 19.05 mm.

In some examples, the height is less than 16.31 mm. In any of these examples, the height can be +/−0.46 mm. In some instances, the solid oral pharmaceutical composition is a tablet.

In some instances, the solid oral pharmaceutical composition is oval or oblong. In some examples, the width of the solid oral pharmaceutical composition is between about 0.1 mm and about 13 mm. For example, the width can be about 0.1 mm, 0.5 mm, 1 mm, 1.5 mm, 2 mm, 2.5 mm, 3 mm, 3.5 mm, 4 mm, 4.1 min, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 ram, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 nun, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, 10.1 mm, 10.2 mm, 10.3 mm, 10.4 mm, 10.5 mm, 10.6 mm, 10.7 mm, 10.8 mm, 10.9 mm, 11 mm, 11.1 mm, 11.2 mm, 11.3 mm, 11.4 mm, 11.5 mm, 11.6 trim, 11.7 mm, 11.8 mm, 1.9 mm, 12 mm, 12.1 mm, 12.2 mm, 12.3 mm, 12.4 mm, 12.5 mm, 12.6 mm, 12.7 mm, 12.8 mm, 12.9 mm, or 13 mm. In some examples, the width is less than or equal to 5.49 mm, between 5.5 mm and 6.05 mm, between 6.06 trim and 6.73 mm, between 6.74 mm and 7.56 mm, between 7.57 mm and 9.19 mm, between 9.20 mm and 11.19 mm, or greater than 11.19 mm. In some examples, the width is less than 5.74 mm. In some examples, the width is less than 6.30 mm. In some examples, the width is less than 6.98 mm. In some examples, the width is less than 7.81 mm. In some examples, the width is less than 9.39 mm. In some examples, the width is less than 11.39 mm. In any of these examples, the width can be +/−0.051 mm. In some instances, the solid oral pharmaceutical composition is a tablet.

In some instances, the solid oral pharmaceutical composition height is less than 16.31 mm and the width is less than 11.39 mm. In some examples, the height is less than 19.05 mm and the width is less than 9.39 mm. In some examples, the height is less than 13.72 mm and the width is less than 9.39 mm. In some examples, the height is less than 15.10 mm and the width is less than 7.81 mm. In some examples, the height is less than 11.20 mm and the width is less than 7.81 mm. In some examples, the height is less than 11.00 mm and the width is less than 6.98 mm, in some examples, the height is less than 10.30 mm and the width is less than 6.30 mm. In some examples, the height is less than 9.40 mm and the width is less than 5.74 mm. In any of these examples, the height can be +/−0.46 mm and the width can be +/−0.051 mm, in some instances, the solid oral pharmaceutical composition is a tablet.

In some cases, a solid oral pharmaceutical composition disclosed herein is over-encapsulated by a capsule. In some instances, the solid oral pharmaceutical composition is a tablet and the tablet is over-encapsulated by a capsule. Such a capsule can have a length of between about 5 mm and about 20 mm. For example, the length of the capsule can be about 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, or 20 mm. In some examples, the capsule length is less than 5 mm. In some examples the capsule length of greater than 20 mm. In some examples, the capsule length is 9.40 mm. In some examples, the capsule length is less 10.30 mm. In some examples, the capsule length is 11.00 mm. In some examples, the capsule length is 11.20 mm. In some examples, the capsule length is 15.10 mm. In some examples, the capsule length is 13.72 mm. In some examples, the capsule length is 19.05 mm. In some examples, the capsule length is 16.31 mm. In any of these examples, the tolerance of the capsule internal diameter can be +/−0.46 mm.

In some instances, the capsule has an internal diameter between about 2. mm and about 15 mm. For example, the internal diameter can be about 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 nun, 9.5 mm, 10 mm, 10.5 mm, 11 mm, 11.5 mm, 12 mm, 12.5 mm, 13 mm, 13.5 mm, 14 mm, 14.5 mm, or 15 mm. In some examples, the internal diameter is less than 2 mm. In some examples the internal diameter is greater than 15 mm. In some examples, the internal diameter is 5.74 mm. In some examples, the internal diameter is 6.30 mm. In some examples, the internal diameter is 6.98 mm, In some examples, the internal diameter is 7.81 mm. In some examples, the internal diameter is 9.39 mm. In some examples, the internal diameter is 11.39 mm. In any of these examples, the tolerance of the capsule internal diameter can be +/−0.051 mm.

In some instances, the capsule has a height of 16.31 mm and an internal diameter of 11.39 mm. In some examples, the tablet is over-encapsulated by a capsule with a height of 19.05 mm and an internal diameter of 9.39 mm. In some examples, the tablet is over-encapsulated by a capsule with a height of 13.72 mm and an internal diameter of 9.39 mm. In some examples, the tablet is over-encapsulated by a capsule with a height of 15.10 mm and an internal diameter of 7.81 mm. In some examples, the tablet is over-encapsulated by a capsule with a height of 11.20 mm and an internal diameter of 7.81 mm. In some examples, the tablet is over-encapsulated by a capsule with a height of 11.00 mm and an internal diameter of 6.98 mm. In some examples, the tablet is over-encapsulated by a capsule with a height of 10.30 mm and an internal diameter of 6.30 mm. In sonic examples, the tablet is over-encapsulated by a capsule with a height of 9.40 mm and an internal diameter of 5.74 mm. In any of these examples, the tolerance of the capsule height can be +/−0.46 min and the tolerance of the internal diameter can be +/−0.051 min.

### Method of Manufacture

In some cases, a pharmaceutical composition disclosed herein is prepared by compressing a first matrix and a second matrix with a press machine, e.g., EP-200L Bi-layer Tablet Press, the first or second matrix of which is prepared by screening ingredients through a mesh (e.g., 10, 20, 30, or 40 mesh screen) in a certain order and then blended to produce a respective matrix formulation. In some instances, the first matrix is prepared with one or more following steps: I) A half of starch, opioid analgesic (e.g., oxycodone HCl), and the other half of the starch are screened in order (e.g., through a 10, 20, 30, or 40 mesh screen) and then blended in a V-blender (e.g., for about 5, 10, 15, 20, 25, 30, 40, 50, or 60 minutes) to generate Blend 1. II) A half of hydroxypropyl methylcellulose, croscarmellose sodium, Blend 1, the other half of the h.ydroxypropyl methylcellulose are screened in order (e.g., through a 10, 20, 30, or 40 mesh screen) and then blended in the V-blender (e.g., for about 5, 10, 15, 20, 25, 30, 40, 50, or 60 minutes) to generate Blend 2. III) A half of microcrystalline cellulose, and mannitol are screened in order (e.g., through a 10, 20, 30, or 40 mesh screen) to produce Mix 1. IV) Blend 2 and the other half of the microcrystalline cellulose are screened in order (e.g., through a 10, 20, 30, or 40 mesh screen) to produce Mix 2. V) Mix 1 and Mix 2 are blended in the V-blender (e.g., for about 5, 10, 15, 20, 25, 30, 40, 50, or 60 minutes) to generate Blend 3. VI) Magnesium stearate and stearic acid are screened in order through a 40 mesh screen to produce Mix 3. VII) Blend 3 and Mix 3 are blended in the V-blender (e.g., for about 5 minutes) to generate the first matrix formulation. In some instances, the second matrix is prepared with one or more following steps: i) A portion of croscarmellose sodium is added to a bag containing an antiemetic (e.g., promethazine HCl) and is mixed manually, e.g., for 1-5 minutes. The mix is emptied from the bag and screened (e.g., through a 10, 20, 30, or 40 mesh screen). ii) The remaining croscarmellose sodium is added to the emptied bag to rinse the bag and then screened (e.g., through a 10, 20, 30, or 40 mesh screen). iii) A portion of silicified microcrystalline cellulose is screened (e.g., through a 10, 20, 30, or 40 mesh screen). iv) The foregoing three groups of screened materials are blended in a V-blender (e.g., for 5, 10, 15. 20, 25, 30, 40, 50, or 60 minutes) to produce Blend 1. v) Blend 1 and the remaining silicified microcrystalline cellulose are screened in order (e.g., through a 10, 20, 30, or 40 mesh screen) and then blended in the V-blender (e.g., for 5, 10, 15, 20, 25, 30, 40, 50, or 60 minutes) to produce Blend 2. vi) Magnesium stearate is screened (e.g., through a 10, 20, 30, or 40 mesh screen) and blended with Blend 2 in the V-blender (e.g., for 5, 10, 15, 20, 25, 30, 40, 50, or 60 minutes) to generate the second matrix.

### Method of Treatment

Described herein are methods for preventing an adverse effect such as nausea, vomiting, other gastric upsets, skin rashes, itching, allergic reactions such as swelling, difficulty breathing, closing of throat, abdominal pain, unusual bleeding or bruising, skin rashes, sedation, CNS depression, or respiratory depression in a subject receiving, or in need of, an opioid analgesic. In some instances, the opioid analgesic is oxycodone or a pharmaceutically acceptable salt thereof. In some instances, the oxycodone or a pharmaceutically acceptable salt thereof is oxycodone hydrochloride. In some instances, the opioid analgesic is hydrocodone or a pharmaceutically acceptable salt thereof. In some instances, the hydrocodone or a pharmaceutically acceptable salt thereof is hydrocodone bitartrate. In some instances, the prevention of an adverse effect is accomplished by the administration of an effective amount of an antiemetic. In some instances, the antiemetic is promethazine or a pharmaceutically acceptable salt thereof. in some instances, the promethazine or a pharmaceutically acceptable salt thereof is promethazine hydrochloride. In some instances, provided herein are methods for preventing or treating pain, comprising administering to a subject in need thereof an effective amount of an opioid analgesic and an agent that reduces side effects of the opioid analgesic. In some instances, the agent that reduces an adverse effect is promethazine. In some instances, provided herein are methods for preventing or treating pain, comprising administering to a subject in need thereof an effective amount of an opioid analgesic, a non-opioid analgesic, and an agent that reduces side effects of the opioid analgesic. In some instances, the agent that reduces an adverse effect is promethazine,

In some cases, an administration is continued for only a relatively short time in the case of an acute condition requiting opioid therapy or for long periods in the case of conditions requiring chronic use of opioid analgesics. In some instances, the dosing of the opioid analgesic is dependent upon the condition being treated, the subject's individual perception of pain and the use of the opioid on a set time schedule as a prophylactic to prevent the onset of pain or on an as needed basis in response to perceived pain. In some instances, the choice of selecting a dosage of a composition that contains suitable amount of promethazine is dependent upon the extent and severity of the adverse effects including nausea, vomiting, other gastric upsets, skin rashes, allergic reactions such as swelling, difficulty breathing, closing of throat, abdominal pain, unusual bleeding or bruising, skin rashes, sedation, CNS depression, or respiratory depression in a subject, upon the sensitivity to side-effect-reducing compounds such as promethazine in a subject, upon the likelihood of subject losing medication by vomiting, and/or on an as needed basis in response to perceived adverse effects. in some instances, the dosage is assessed by a prescribing professional evaluating the subject, the condition treated, the analgesic to be used, diet and the expected duration of therapy.

In some cases, provided herein is a method for treating or preventing pain in a subject in need thereof comprising administering a pharmaceutical composition, such as a solid oral pharmaceutical composition, comprising an effective amount of an opioid analgesic to treat or prevent pain in the subject in need thereof and an effective amount of an antiemetic to reduce or prevent an adverse event associated with the opioid analgesic in the subject in need thereof. In some instances, the adverse effect comprises nausea, vomiting, other gastric upsets, skin rashes, allergic reactions such as swelling, difficulty breathing, closing of throat, itching, abdominal pain, unusual bleeding or bruising, skin rashes, sedation, CNS depression, or respiratory depression. in some instances, the adverse effect comprises nausea and vomiting. In some instances, the adverse effect is nausea or vomiting.

It is believed that administration of a pharmaceutical composition disclosed herein would result in treatment of the subject which includes elimination or reduction of an adverse effect associated with opioid analgesics and enhance the beneficial uses of such analgesics. In some instances, such an adverse effect otherwise renders administration of certain analgesics intolerable, due to for example vomiting, nausea, and skin rashes. In some instances, provided herein are methods directed to target populations of subjects that are susceptible to such an adverse effect(s), thus allowing such subjects to benefit from the pain-alleviating effects of analgesic-based pain relief, administration of which would otherwise be intolerable. In some instances, the pharmaceutical composition is a solid oral pharmaceutical composition.

For example, by reducing the risk of vomiting, the risk of subject losing the analgesics (and losing the pain-relieving beneficial effects of analgesics) by vomiting is minimized. In some instances, administration is adjusted to provide the dose of side-effect-reducing compound to match the subject's analgesic ingestion without separate intervention by the health care professionals. Adding one or more additional active agents, such as promethazine, to the present compositions is believed to result in a composition having reduced potential for abuse and diversion.

### Treatment or Prevention of Pain

The present compositions and methods are useful for treating, reducing or preventing pain in a subject in need thereof. In some instances, provided herein are methods for treating or preventing pain, comprising administering to a subject in need thereof a pharmaceutical composition disclosed herein, such as a solid oral pharmaceutical composition disclosed herein. Pain treatable or preventable includes, but is not limited to, pain associated with cancer, chronic or acute pain, headache pain, migraine headache, chronic headache, surgical procedure, acute or chronic physical injury, bone fracture or crush injuries, spinal cord injury, inflammatory disease (e.g., pancreatitis), noninflammatory neuropathic or dysfunctional pain conditions, or a combination thereof.

In some cases, methods of treatment or prevention comprising administering a composition are for treating pain or preventing pain. In some instances, the pain treatable or preventable via administration of a pharmaceutical composition disclosed herein includes but is not limited to headache pain, and/or headache related symptoms as further described herein below. In some instances, the pharmaceutical composition is a solid oral pharmaceutical composition.

### Treatment or Prevention of Headache

The present pharmaceutical compositions and methods are useful for treating, reducing or preventing a headache in a subject in need thereof. Preventable or treatable headaches include but are not limited to migraine headaches (with or without aura), cluster headaches, chronic headaches, tension type headaches, Hemicrania. Continua, new daily persistent, chronic tension type headaches or any combination thereof. In some instances, a method for treating or preventing a headache comprises administering to a subject in need thereof a pharmaceutical composition disclosed herein. In some instances, the pharmaceutical composition is a solid oral pharmaceutical composition. Migraines and cluster headaches are both important, well-known, and extensively studied medical problem. In many cases, they completely incapacitate a sufferer for the duration of the headache.

In some cases, a pharmaceutical compositions disclosed herein are administered to a subject to treat, eliminate or prevent at least one headache symptom. An effective amount is a dosage sufficient to reduce at least one symptom associate with a headache. Headache symptoms include: (1) frequency, which can be evaluated over a span of time, such as number of such headaches per week, per month, or per year; (2) duration, which evaluates (usually in hours) how long a headache lasts, from the time it begins to develop into a migraine or cluster headache, until it has been resolved; and (3) severity (also referred to as intensity), which is based on subjective estimates of the severity or intensity of pain or other symptoms (such as nausea) being suffered by patients during such headaches. In some instances, the pharmaceutical compositions are used in a method to reduce the frequency, duration or severity of a preventable or treatable headache. In some instances, the pharmaceutical composition is a solid oral pharmaceutical composition.

### Treatment or Prevention of Photophobia

In some cases, provided herein are pharmaceutical compositions and methods for treating or preventing photophobia. In some instances, the composition comprises an effective amount of each of an opioid analgesic and an antiemetic, as disclosed herein above. In some instances, the antiemetic is promethazine or a pharmaceutically acceptable salt thereof and the opioid analgesic is oxycodone or a pharmaceutically acceptable salt thereof. In some instances, the antiemetic is promethazine or a pharmaceutically acceptable salt thereof and the opioid analgesic is hydrocodone or a pharmaceutically acceptable salt thereof. In some instances, the photophobia is associated with a migraine headache. In some instances, the pharmaceutical composition is a solid oral pharmaceutical composition.

### EXAMPLES

### Example 1

### Bi-Layered Tablets: Oxycodone and Promethazine

Bi-layered tablets were designed, each including the active ingredients and amounts as shown in Table 1.

TABLE 1

| INGREDIENT | QUANTITY/TABLET (MG) |
|---|---|
| Oxycodone Hydrochloride | 5 |
| Promethazine Hydrochloride | 12.5 |

Bi-layered tablets comprising different excipient ingredient combinations were manufactured. The ingredient list and amounts for manufactured Composition 1A and 1B are shown in Tables 2-3.

TABLE 2
First Layer—Composition 1A:

| | QUANTITY/TABLET | CONCENTRATION |
|---|---|---|
| INGREDIENT | (MGS) | % W/W |
| Oxycodone hydrochloride | 5 | 1.43 |
| Croscarmellose Sodium | 6.67 | 1.90 |
| (AcDiSol) | | |
| Silicified Microcrystalline | 117.33 | 33.52 |
| Cellulose (Prosolv HD90) | | |
| Hydroxypropyl | 10.33 | 2.95 |
| methylcellulose (Methocel | | |
| K4M) | | |
| Magnesium Stearate | 1 | 0.29 |
| Stearic Acid | 1 | 0.29 |
| Lactose Monohydrate | 58.67 | 16.76 |
| (Tablettose 70) | | |
| LAYER TOTAL | 200 | 57.14 |

Second Layer—Composition 1A:

| | QUANTITY/TABLET | CONCENTRATION |
|---|---|---|
| INGREDIENT | (MG) | % W/W |
| Promethazine hydrochloride | 12.5 | 3.57 |
| Silicified Microcrystalline | 121.5 | 34.71 |
| Cellulose (Prosolv HD90) | | |
| Croscarmellose Sodium | 15 | 4.29 |
| (AcDiSol) | | |
| Magnesium Stearate | 1 | 0.29 |
| LAYER TOTAL | 150 | 42.86 |
| TABLET TOTAL | 350 | 100 |

TABLE 3
First Layer—Composition 1B:

| | QUANTITY/TABLET | CONCENTRATION |
|---|---|---|
| INGREDIENT | (MGS) | % W/W |
| Oxycodone hydrochloride | 5 | 1.43 |
| Croscarmellose Sodium | 6.67 | 1.9 |
| (AcDiSol) | | |
| Silicified Microcrystalline | 120.49 | 34.43 |
| Cellulose (Prosolv HD90) | | |
| Hydroxypropyl | 5.6 | 1.6 |
| methylcellulose (Methocel | | |
| K4M) | | |
| Magnesium Stearate | 1 | 0.29 |
| Stearic Acid | 1 | 0.29 |
| Lactose Monohydrate | 60.24 | 17.21 |
| (Tablettose 70) | | |
| LAYER TOTAL | 200 | 57.14 |

Second Layer—Composition 1B:

| | QUANTITY/TABLET | CONCENTRATION |
|---|---|---|
| INGREDIENT | (MG) | % W/W |
| Promethazine hydrochloride | 12.5 | 3.57 |
| Silicified Microcrystalline | 121.5 | 34.71 |
| Cellulose (Prosolv HD90) | | |
| Croscarmellose Sodium | 15 | 4.29 |
| (AcDiSol) | | |
| Magnesium Stearate | 1 | 0.29 |
| LAYER TOTAL | 150 | 42.86 |
| TABLET TOTAL | 350 | 100 |

### Example 2

### Dissolution of Compositions 1A and 1B

Dissolution apparatus was a USP Rotating Paddle Apparatus 2 with an automated sampling station (e.g., VK-8000 or equivalent). Dissolution fluid was 900 mL of de-aerated 0.01 N HCl, maintained at 37.0+/−0.5° C. during dissolution procedure. The fluid was prepared by diluting 5 mL of concentrated HCl in 6000 mL of de-aerated water, and mixed. To measure peaks, a dual wavelength detector (e.g., Hitachi L-2420) was used, or alternatively, two separate chromatographic systems can be used in order to measure the peaks at two different wavelengths.

Standard Solution Preparation: Each ingredient was weighed (oxycodone hydrochloride and promethazine hydrochloride) into a 50 mL volumetric flask, and diluted to volume with dissolution media. The resulting solution was mixed to form a stock solution. 2. mL each of stock standard solutions were diluted with dissolution fluid and mixed to produce a final standard solution.

Dissolution test solutions were prepared in 900 mL of 0.01 N HCl using the USP Rotating Paddle Apparatus at 50 WM. An aliquot of the dissolution solution was filtered and a 50-pL aliquot was chromatographed on a 50-mm×4.6-mm (i.d.) Waters sunFire™ C₁₈, 3.5-μm particle size column using a gradient HPLC method. Mobile phase A consisted of waterlacetonitrile/TFA, 950/50/2 (v/v/v) and mobile phase B consisted of waterlacetonitrile/TFA, 50/950/1.5 (v/v/v). The flow rate was 2.0 mL/minute. For example, the amount of oxycodone released was determined at 230 nm by comparing the area obtained for the peak due to oxycodone in the chromatogram of the dissolution test solution to that obtained for the corresponding peak in a chromatogram of a standard solution. The amount of promethazine released was determined at 230 nm by comparing the area obtained for the peak due to promethazine the chromatogram of the dissolution test solution to that obtained for the corresponding peak in a chromatogram of a standard solution.

Paddle speed was 50 rpm; pull volume was 10 mL (no replacement); Pull points: 5, 10, 15, 30, and 60 minutes. The amount of each component dissolved in the dissolution medium was determined by HPLC. The method can use a high purity, bonded C18 stationary phase and a binary mobile phase consisting of an appropriate buffer and organic modifier.

Dissolution Procedure. 900 mL of dissolution fluid preheated to 37° C. was placed into each vessel. Tablets of Analgesic Composition (Example 1, Tables 2 and 3) above were weighed and placed in vessels respectively. At prescribed time intervals, 5 mL aliquot of the dissolution fluid was drawn using the automated sampling station equipped with a 35 μm fill flow filter connected to a sampling probe. Filtrate was allowed to cool to room temperature, to produce a final sample solution. Fluid withdrawn was not replaced. Samples were injected in HPLC for analysis after a baseline was established. Peak area responses were measured for each component. The resolution between each peak was calculated, as well as the tailing factor. The mean and %RSD values for promethazine and oxycodone were measured at 230 nm. The five replicate injections were not more than 2.0% RSD. 50 μL aliquots of standard and sample solutions were subjected to liquid chromatography.

The amount of a pharmaceutically active agent in a tablet was determined by comparing the area obtained for the peak due to the agent in a chromatogram of the dissolution test solution to that obtained for the corresponding peak in a chromatogram of a standard solution. See Tables 4-5.

TABLE 4
Oxycodone and Promethazine Dissolution
Profile Results for Composition 1A.

| Ingredient | 5 min | 10 min | 15 | 30 | 60 |
|---|---|---|---|---|---|
| Oxycodone | 62.1% | 72.6% | 76.5% | 83.3% | 92.6% |
| Promethazine | 81.4% | 85.9% | 86.5% | 88.0% | 88.0% |

TABLE 5
Oxycodone and Promethazine Dissolution Profile
Results for Composition 1B.

| Ingredient | 5 min | 10 min | 15 | 30 | 60 |
|---|---|---|---|---|---|
| Oxycodone | 78.3% | 84.2% | 87.1% | 92.1% | 97.9% |
| Promethazine | 77.0% | 78.6% | 81.5% | 87.2% | 87.2% |

### Example 3

### Bi-Layered Tablets: Oxycodone and Promethazine

Solid oral pharmaceutical bi-layer tablets comprising Compositions 2A and 2B as shown in Table 6 were manufactured.

TABLE 6
First Layer—Composition 2A:

| | QUANTITY/TABLET | CONCENTRATION |
|---|---|---|
| INGREDIENT | (MGS) | % W/W |
| Oxycodone hydrochloride | 5 | 3.03% |
| Microcrystalline Cellulose | 27.65 | 16.76% |
| (Prosolv HD90) | | |
| Hydroxypropyl | 1 | 0.61% |
| methylcellulose (Methocel | | |
| K4M) | | |
| Magnesium Stearate | 0.175 | 0.11% |
| Stearic Acid | 0.175 | 0.11% |
| Sodium starch glycolate | 1 | 0.61% |

Second Layer—Composition 2A:

| | QUANTITY/TABLET | CONCENTRATION |
|---|---|---|
| INGREDIENT | (MG) | % W/W |
| Promethazine hydrochloride | 12.5 | 7.58% |
| Microcrystalline Cellulose | 101.5 | 61.52% |
| (Prosolv HD90) | | |
| Croscarmellose Sodium | 15 | 9.09% |
| (AcDiSol) | | |
| Magnesium Stearate | 1 | 0.61% |

First Layer—Composition 2B:

| | QUANTITY/TABLET | CONCENTRATION |
|---|---|---|
| INGREDIENT | (MGS) | % W/W |
| Oxycodone hydrochloride | 5 | 3.03% |
| Microcrystalline | 27.15 | 16.45% |
| Cellulose(Prosolv HD90) | | |
| Hydroxypropyl | 2 | 1.21% |
| methylcellulose (Methocel | | |
| K4M) | | |
| Magnesium Stearate | 0.175 | 0.11% |
| Stearic Acid | 0.175 | 0.11% |
| Sodium starch glycolate | 0.5 | 0.30% |

Second Layer—Composition 2B:

| | QUANTITY/TABLET | CONCENTRATION |
|---|---|---|
| INGREDIENT | (MG) | % W/W |
| Promethazine hydrochloride | 12.5 | 7.58% |
| Microcrystalline | 101.5 | 61.52% |
| Cellulose(Prosolv HD90) | | |
| Croscarmellose Sodium | 15 | 9.09% |
| (AcDiSol) | | |
| Magnesium Stearate | 1 | 0.61% |

### Example 4

### Dissolution of Compositions 2A and 2B

Dissolution apparatus was a USP Rotating Paddle Apparatus 2 with an automated sampling station (e.g., VK-8000 or equivalent). Dissolution fluid was 900 mL of dc-aerated 0.01 N HCl, maintained at 37.0+/−0.5° C. during dissolution procedure. The fluid was prepared by diluting 5 mL of concentrated HCl in 6000 mL of de-aerated water, and mixed. To measure peaks, a dual wavelength detector (e.g., Hitachi L-2420) was used, or alternatively, two separate chromatographic systems can be used in order to measure the peaks at two different wavelengths.

Standard Solution Preparation: Each ingredient was weighed (oxycodone hydrochloride and promethazine hydrochloride) into a 50 mL volumetric flask, and diluted to volume with dissolution media. The resulting solution was mixed to form a stock solution. 2 mL each of stock standard solutions were diluted with dissolution fluid and mixed to produce a final standard solution.

Dissolution test solutions were prepared in 900 mL of 0.01 N HCl using the USP Rotating Paddle Apparatus at 50 WM. An aliquot of the dissolution solution was filtered and a 50-pL aliquot was chromatographed on a 50-mm×4.6-mm (i.d.) Waters sunFire™ C₁₈, 3.5-μm particle size column using a gradient HPLC method. Mobile phase A consisted of waterlacetonitrile/TFA, 950/50/2 (v/v/v) and mobile phase B consisted of waterlacetonitrile/TFA, 50/950/1.5 (v/v/v). The flow rate was 2.0 mL/minute. For example, the amount of oxycodone released was determined at 230 nm by comparing the area obtained for the peak due to oxycodone in the chromatogram of the dissolution test solution to that obtained for the corresponding peak in a chromatogram of a standard solution. The amount of promethazine released was determined at 230 nm by comparing the area obtained for the peak due to promethazine the chromatogram of the dissolution test solution to that obtained for the corresponding peak in a chromatogram of a standard solution.

Paddle speed was 50 rpm; pull volume was 10 mL (no replacement); Pull points: 5, 10, 15, 20, 25, 30, 45 and 60 minutes. The amount of each component dissolved in the dissolution medium was determined by HPLC. The method can use a high purity, bonded C18 stationary phase and a binary mobile phase consisting of an appropriate buffer and organic modifier.

Dissolution Procedure. 900 mL of dissolution fluid preheated to 37° C. was placed into each vessel. Tablets of Analgesic Composition (Example 4) above were weighed and placed in vessels respectively. At prescribed time intervals, 5 mL aliquot of the dissolution fluid was drawn using the automated sampling station equipped with a 35 μm full flow filter connected to a sampling probe. Filtrate was allowed to cool to room temperature, to produce a final sample solution. Fluid withdrawn was not replaced. Samples were injected in HPLC for analysis after a baseline was established. Peak area responses were measured for each component. The resolution between each peak was calculated, as well as the tailing factor. The mean and % RSD values for promethazine and oxycodone were measured at 230 nm. The five replicate injections were not more than 2.0% RSD. 50 μL aliquots of standard and sample solutions were subjected to liquid chromatography.

The amount of a pharmaceutically active agent in a tablet was determined by comparing the area obtained for the peak due to the agent in a chromatogram of the dissolution test solution to that obtained for the corresponding peak in a chromatogram of a standard solution. See Tables 7-11.

TABLE 7
Oxycodone and Promethazine Dissolution Profile Results for Composition 2A.

| | 5 min | 10 min | 15 | 30 | 45 |
|---|---|---|---|---|---|
| | Average % | Average % | Average % | Average % | Average % |
| | (low %- | (low %- | (low %- | (low %- | (low %- |
| Ingredient | high %) | high %) | high %) | high %) | high %) |
| Oxycodone | 82% | 85.7% | 88.1% | 92% | 93% |
| | (74-93.2%) | (78.2-94.8%) | (80.3-95.4%) | (84.5-99.3%) | (85.6-100.9%) |
| Promethazine | 88.9% | 93% | 95.1% | 98.7% | 100% |
| | (76.1-101.6%) | (80.1-105.2%) | (82.7-106%) | (91.1-107.8%) | (96.4-108%) |

TABLE 8
Oxycodone and Promethazine Dissolution Profile Results for
Composition 2A at t = 0 month

| | 15 | 30 | 45 | 60 |
|---|---|---|---|---|
| | Average % | Average % | Average % | Average % |
| | (low %- | (low %- | (low %- | (low %- |
| Ingredient | high %) | high %) | high %) | high %) |
| Oxycodone | 86.3% | 94.3% | 96.4% | 96.7% |
| | (58.9-95.8%) | (89.4- | (88.6- | (89.7- |
| | | 97.1%) | 102.1%) | 100.9%) |
| Promethazine | 86.3% | 91.7% | 93.2% | 94.7% |
| | (84.9-103.5%) | (85.7- | (87.9- | (83.5- |
| | | 102.1%) | 100.4%) | 100.8%) |

TABLE 9
Oxycodone and Promethazine Dissolution Profile Results for
Formulation 2A at t = 1 month

| | 15 | 30 | 45 | 60 |
|---|---|---|---|---|
| | Average % | Average % | Average % | Average % |
| | (low %- | (low %- | (low %- | (low %- |
| Ingredient | high %) | high %) | high %) | high %) |
| Oxycodone | 90.3% | 92.6% | 94.3% | 94.1% |
| | (80.7-97.8%) | (87.3-97.5%) | (89.8-98.6%) | (90.6-96.6%) |
| Promethazine | 91.0% | 94.1% | 97.8% | 98.4% |
| | (80.0-97.5%) | (87.1-98.3%) | (91.2-102.4%) | (94.4-102.4%) |

TABLE 10
Oxycodone and Promethazine Dissolution Profile Results for
Formulation 2A at t = 2 months

| | 5 | 10 | 15 | 30 |
|---|---|---|---|---|
| Ingredient | Average % | Average % | Average % | Average % |
| Oxycodone | 82% | 87% | 91% | 96% |
| Promethazine | 90% | 97% | 99% | 102% |

TABLE 11
Oxycodone and Promethazine Dissolution Profile Results for
Formulation 2A at t = 3 months

| | 5 | 10 | 15 | 30 |
|---|---|---|---|---|
| | Average % | Average % | Average % | Average % |
| | (low %- | (low %- | (low %- | (low %- |
| Ingredient | high %) | high %) | high %) | high %) |
| Oxycodone | 102% | 102% | 102% | 102% |
| | (98.1-103.7%) | (99.8- | (99.1- | (100.5- |
| | | 103.6%) | 103.4%) | 103.9%) |
| Promethazine | 100% | 101% | 101% | 101% |
| | (96.7-104.2%) | (98.1- | (97.6- | (97.1- |
| | | 106.5%) | 106.1%) | 105.6%) |

### Example 5

### Coating Layer with Abuse Deterrent Gelling Agent

A coating layer comprising an abuse deterrent gelling agent was manufactured having ingredients listed in Table 12.

### Example 6

### Hydrocodone/Promethazine Bi-Layered Tablet

A solid oral pharmaceutical bi-layer tablet composition (Composition 3) was manufactured, having ingredients listed in Table 13.

TABLE 13
First Layer—Composition 3:

| | QUANTITY/TABLET | CONCENTRATION |
|---|---|---|
| INGREDIENT | (MG) | % W/W |
| Hydrocodone bitartrate | 75 | 1.13% |
| Acetaminophen | 325 | 48.95% |
| Croscarmellose Sodium | 10 | 1.51% |
| Microcrystalline Cellulose | 150.4 | 22.65% |
| Hydroxypropyl | 15.5 | 2.33% |
| methylcellulose | | |
| Magnesium Stearate | 2.75 | 0.41% |
| Stearic Acid | 2.75 | 0.41% |

Second Layer—Composition 3:

| | QUANTITY/TABLET | CONCENTRATION |
|---|---|---|
| INGREDIENT | (MG) | % W/W |
| Promethazine hydrochloride | 12.5 | 1.88% |
| Microcrystalline Cellulose | 121.5 | 18.30% |
| Croscarmellose Sodium | 15 | 2.26% |
| Magnesium Stearate | 1 | 0.15% |

### Example 7

### Abuse-Deterrent Coating Materials

To assess abuse deterrent material for incorporation into a solid oral pharmaceutical coating, three different grades of Polyethylene Oxide were evaluated. These PEO grades (100,000 molecular weight (PEO-1), 200,000 molecular weight (PEO-2), and 7,000,000 molecular weight (PEO-3) were dispersed in varying amounts into 10 mL of water to assess abuse deterrent potential of each grade. A summary of results is shown in Table 14.

TABLE 14

| PEO (approx. | | | | |
|---|---|---|---|---|
| molecular | Quantity of PEO added to 10 mL of water | | | |
| weight) | 21 mg | 35 mg | 70 mg | 105 mg |
| PEO-1 | Low | Low | Low | Low |
| (100,000 MW) | viscosity | viscosity | viscosity | viscosity |
| | solution | solution | solution | solution |
| PEO-2 | Low viscosity | Low viscosity | Low viscosity | Thin gel |
| (200,000 MW) | solution | solution | solution | |
| PEO-3 | Thin gel | Thin gel | Thin gel | Thin gel |
| (7,000,000 MW) | | | | |

For those grades and concentrations of PEO that produced a gel consistency, a. syringeability assay was performed. This assay assessed whether or not the aqueous solution of the polymers could be drawn into a 10 mL syringe through an 18 gauge needle when filtered through a small piece of cotton. Results of this syringeability assay are summarized in Table 15.

TABLE 15

| | 105 mg | 21 mg | 35 mg | 70 mg | 105 mg |
|---|---|---|---|---|---|
| Sample | PEO-2 | PEO-3 | PEO-3 | PEO-3 | PEO-3 |
| Syringed | Yes | Difficult | No | No | No |
| through | | | | | |
| cotton | | | | | |

PEO-3 was assayed for preventing extraction in different, commonly available, extraction solvents. The solvents were Vinegar (5% acetic acid solution), 95% Ethanol (190 proof), 70% Isopropyl Alcohol, and 50% Ethanol (100 proof). The appearance of the solutions formed by the addition of different levels of PEO-3 to the solvents was noted, followed by a syringeability assessment. Tables 16 and 17 describe results from these assays. An amount of 21 mg to 35 mg of polymer gives was successful across the range of potential extraction solvents.

TABLE 16
Solution appearance for PEO-3 in various solvents

| | Quantity of PEO added to 10 mL of water | | | |
|---|---|---|---|---|
| Solvent | 21 mg | 35 mg | 70 mg | 105 mg |
| 5% Acetic acid | Thin gel | Thin gel | Thin gel | Thin gel |
| (vinegar) | | | | |
| 95% ethanol | Low | Low | Low | Low |
| (190 proof) | viscosity | viscosity | viscosity | viscosity |
| | w/ | w/ | w/ | w/ |
| | undissolved | undissolved | undissolved | undissolved |
| | polymer | polymer | polymer | polymer |
| 70% isopropyl | Thin gel | Thin gel | Thin gel | Thin gel |
| Alcohol | | | | |
| 50% ethanol | Thin gel | Thin gel | Thin gel | Thin gel |
| (100 proof) | | | | |

TABLE 17
Syringeability assay for PEO-3 in various solvents

| | Quantity of PEO added to 10 mL of water | | | |
|---|---|---|---|---|
| Solvent | 21 mg | 35 mg | 70 mg | 105 mg |
| 5% Acetic | No | No | No | No |
| acid (vinegar) | | | | |
| 95% ethanol | Yes | Yes | Yes | Difficult |
| (190 proof) | | | | |
| 70% isopropyl | No | No | No | No |
| Alcohol | | | | |
| 50% ethanol | No | No | No | No |
| (100 proof) | | | | |

A series of small-scale coating formulations were compounded to assess PEO-3 levels for incorporation into the film coating formulation. An ethanol-based coating formulation was developed so that PEO would be suspended as a solid in the coating dispersion. Triethyl Citrate (TEC) and Polyethylene Glycol 400 (PEG 400) were used for the formulation evaluation. Hydroxypropyl Cellulose (HPC) was chosen as the film-forming polymer as it has better solubility in ethanol.

Small-scale coating solutions were prepared at four PEO-3 concentrations (40%, 50%, 60%, and 70% wlw relative to the total solids in the coating) and three plasticizer levels (10%, 15%, and 20% w/w relative to the HPC) for each of the two plasticizers assayed giving a total of 24 coating formulations. Talc was incorporated into each of the coatings at the level of 50% w/w relative to the HPC content. The dried basis coating composition for each of the coating formulations is given in Tables 18 and 20. Each coating dispersion was compounded in absolute ethanol with a solids content ranging from ˜22% to ˜25% (w/w). An aliquot of each final solution was added to separate aluminum pans and allowed to dry. The resulting films were evaluated visually for appearance, adhesion, and flexibility by flexing the pans to see if the film peels off the pan or cracks. The results of this evaluation are given in Tables 19 and 21.

TABLE 19
Assessment of films cast from TEC coating formulations

| Plasticizer | PEO solid concentration | | | |
|---|---|---|---|---|
| level | 40% | 50% | 60% | 70% |
| 10% | Rough | Rough | Rough Film w/ | Rough Film w/ |
| | Continuous | Continuous | Some Holes. | Holes. |
| | Film. | Film. | Good Adhesion. | Moderate |
| | Good | Good | Slight Cracking | Adhesion. |
| | Adhesion. | Adhesion. | When Pan is | Cracked/Flaked |
| | Good | Good | Flexed. | When Pan is |
| | Flexibility. | Flexibility. | | Flexed. |
| 15% | Rough | Rough | Rough Film w/ | Rough Film w/ |
| | Continuous | Continuous | Some Holes. | Some Holes. |
| | Film. | Film. | Good Adhesion. | Moderate |
| | Good | Good | Slight Cracking | Adhesion. |
| | Adhesion. | Adhesion. | When Pan is | Slight Crack |
| | Good | Good | Flexed. | When Pan is |
| | Flexibility. | Flexibility. | | Flexed. |
| 20% | Rough | Rough | Rough Film w/ | Rough Film w/ |
| | Continuous | Continuous | Some Holes. | Some Holes. |
| | Film. | Film. | Good Adhesion. | Moderate/Good |
| | Good | Good | Very Slight | Adhesion. |
| | Adhesion. | Adhesion. | Cracking | Very Slight |
| | Good | Good | When Pan is | Crack |
| | Flexibility. | Flexibility. | Flexed. | When Pan is |
| | | | | Flexed. |

TABLE 21
Assessment of films cast from PEG 400 coating formulations

| Plasticizer | PEO solid concentration | | | |
|---|---|---|---|---|
| level | 40% | 50% | 60% | 70% |
| 10% | Rough | Rough | Rough Film w/ | Rough Film w/ |
| | Continuous | Continuous | Some Holes. | Holes. |
| | Film. | Film. | Good Adhesion. | Moderate |
| | Good | Good | Slight Cracking | Adhesion. |
| | Adhesion. | Adhesion. | When Pan is | Cracked When |
| | Good | Good | Flexed. | Pan is Flexed. |
| | Flexibility. | Flexibility. | | |
| 15% | Rough | Rough | Rough | Rough Film w/ |
| | Continuous | Continuous | Continuous | Some Holes. |
| | Film. | Film. | Film. | Moderate |
| | Good | Good | Good Adhesion. | Adhesion. |
| | Adhesion. | Adhesion. | Good Flexibility. | Cracked/Flaked |
| | Good | Good | | When Pan is |
| | Flexibility. | Flexibility. | | Flexed. |
| 20% | Rough | Rough | Rough Film w/ | Rough Film w/ |
| | Continuous | Continuous | Some Holes. | Some Holes. |
| | Film. | Film. | Good Adhesion. | Moderate |
| | Good | Good | Slight Cracking | Adhesion. |
| | Adhesion. | Adhesion. | When Pan is | Cracked/Flaked |
| | Good | Good | Flexed. | When Pan is |
| | Flexibility. | Flexibility. | | Flexed. |

The 70% PEO-3 coating formulation containing 20% triethyl citrate (relative to HPC) was chosen for further coating evaluations. Coating trials were conducted on tablet cores formulated to have rapid disintegration times of less than 20 seconds. Prolonged disintegration times for the coated cores are therefore taken as an indication of a possible delay in the drug substance release. The coated cores were evaluated for disintegration time in water and abuse deterrent efficacy by dissolving in 10 mL of extraction solvent and subsequent sytingeability evaluation as described previously. Table 22 shows the coating dispersion formulation that was prepared and Table 23 shows the coated tablet composition that was prepared. Table 24 gives the disintegration test results.

TABLE 22
Coating dispersion Foil-Titillation

| Ingredient | % w/w |
|---|---|
| PEO-3 | 21.0 |
| Hydroxypropyl Cellulose HPC | 5.3 |
| (Nisso SSL SFP) | |
| Triethyl Citrate | 1.06 |
| Tale (Luzanac Pharma M) | 2.64 |
| Ethanol, dehydrated, 200 proof | 70.0 |
| Total | 100.0 |

Disintegration testing was performed on the coated tablet of Table 23, as well as four other coating levels, in 900 ml, of water at 37 degrees Celsius using a USP disintegration apparatus. Table 24 reports the disintegration times for each of the different coating level samples and the uncoated cores for comparison.

TABLE 24

| | Sample | | | | | |
|---|---|---|---|---|---|---|
| | Uncoated | 30 mg | 40 mg | 45 mg | 50 mg | 60 mg |
| | cores | Coating | Coating | Coating | Coating | Coating |
| Disintegration | 00:18 | 21:44 | 35:04 | 44:45 | 55:07 | 59:03 |
| time (mm:ss) | | | | | | |

Coated tablets were also assayed for using the same syringeability test discussed earlier and results are shown in Table 25. The placebo tablet cores disintegrated in more aqueous extraction media (water and vinegar) and not in the organic solvent media (isopropanol and ethanol) (data not shown).

TABLE 25
Syringeability testing through cotton

| | Coating weight gain level | | | |
|---|---|---|---|---|
| Solvent | 30 mg | 40 mg | 45 mg | 50 mg |
| Water | No | No | No | No |
| 70% Isopropyl Alcohol | No | No | No | No |
| 5% Acetic Acid (Vinegar) | No | No | No | No |
| 50% Ethanol (100 proof) | No | No | No | No |

### Example 8

### Sub-Soating Materials

A tablet formulation was manufa.ctured having a sub-coating below the PEO coat. Ingredients and amount per table are provided in Table 26. Tablet cores were sub-coated and coated to varying thicknesses summarized in Table 27.

TABLE 27

| | Coating Weight Gain | | | | | |
|---|---|---|---|---|---|---|
| | 10 mg | 15 mg | 20 mg | 25 mg | 30 mg | 40 mg |
| Avg. Approx. | 100 | 140 | 150 | 185 | 205 | 255 |
| Coating | | | | | | |
| thickness (μm) | | | | | | |

The tablets with sub-coating and coatings were assessed for disintegration time compared to uncoated cores (Table 28), by adding the tablets to 900 mL of water at 37° C. (±2° C.) using a USP disintegration apparatus. Syringeability was also assessed as described previously and the results are summarized in Table 29.

TABLE 28

| | Coating on core with sub-coating | | | | | | |
|---|---|---|---|---|---|---|---|
| | Uncoated | | | | | | |
| | core | 10 mg | 15 mg | 20 mg | 25 mg | 30 mg | 40 mg |
| Disin- | 0 min | 3 min | 4 min | 11 min | 11 min | 11 min | 16 min |
| tegration | 18 sec | 26 sec | 39 sec | 35 sec | 37 sec | 49 sec | 02 sec |
| time | | | | | | | |

TABLE 29

| | Coating Weight Gain Level | | | | | |
|---|---|---|---|---|---|---|
| Solvent | 10 mg | 15 mg | 20 mg | 25 mg | 30 mg | 40 mg |
| Water | No | No | No | No | No | No |
| 70% | No | No | No | No | No | No |
| isopropyl | | | | | | |
| alcohol | | | | | | |
| 5% acetic | Difficult | Difficult | Difficult | Difficult | No | No |
| acid | | | | | | |
| 50% | Difficult | No | No | No | No | No |
| ethanol | | | | | | |
| (100 proof) | | | | | | |

### Example 9

### Coated Bi-Layered Tablets

Solid oral pharmaceutical bi-layer tablets comprising Compositions 1A, 1B, 2A, 2B, and 3A are each separately prepared with a coating layer as shown in Table 12.

### Example 10

### Administration of Bi-Layered Tablets

Any of the compositions of Examples 1, 3, 6, or 7 are orally administered with water to a subject having a tendency to exhibit adverse effects of opioid administration, such as gastric upset, nausea, vomiting, skin rash, sedation, CNS depression, or respiratory depression.

### Example 11

### Manufacture of Bi-Layered Tablets

A batch of solid oral pharmaceutical bi-layer tablets of Table 30 was manufactured, using an amount of material at each step as listed in the “Batch” column of Table 31 and Table 32. Manufacturing was performed by the following protocol. Promethazine (item 1) and croscarmellose sodium (item 2) were mixed together and passed through a #20 mesh screen. A second dispersion of croscarmellose (item 3) was passed through the same screen and added to the previous mixture. Silicified microcrystalline cellulose (item 4) was passed through the same screen and added to the mixture. The mixtures was transferred to a V-shell mixer and blended for 10 minutes. The blended mixture was passed through a #40 mesh sieve. A second dispersion of silicified microcrystalline cellulose (item 5) was passed through the same #40 mesh sieve and then added to the added to the V-shell mixer and blended for 20 minutes. Magnesium stearate (item 6) was passed through the same #40 mesh sieve and then added to the V-shell mixer and blended for 5 minutes. The blended mixture containing items 1-6 (Part A) was then emptied from the blender. The following materials were then dispensed and passed through a #20 mesh sieve in the following order: approximately half of the mannitol (item 10), hydroxypropyl methylcellulose (item 9), croscarmellose sodium (item 8), oxycodone (item 7), and the remainder of the mannitol (item 10). This mixture was transferred to a V-shell blender and mixed for 10 minutes (250 revolutions). The blended mixture was then passed through a #40 mesh sieve and then places back in the V-shell blender and mixed for another 10 minutes. The blended mixture was again passed through a #40 mesh sieve. Silicified microcrystalline cellulose (item 11) was then passed through the same #40 mesh sieve and added to the mixture. The mixture was transferred to the V-shell blender and mixed for 20 minutes (500 revolutions). Magnesium stearate (item 12) and stearic acid (item 13) were passed through a #40 mesh screen and added to the blender and blended for 5 minutes. This blended mixture containing items 7-13 (Part B) was then emptied from the blender.: Flexitab tablet press with ⅜ inch round standard concave tooling (plain faced) and layer 1 and 2 hoppers were assembled. An appropriate amount of Part A was added to hopper 1 and an appropriate amount of Part B was added to hopper 2, The press was dialed in to give 150 mg layer 1 tablets. Layer 2 blend was added to the layer 1 tablet to give a layer 2 weight of 200 mg, for a total tablet weight of 350 mg and a final tablet hardness of 8-10 kp.

TABLE 30
Bi-layer Tablet

| | | | |
|---|---|---|---|
| | | Con- | Amount/ |
| Item | | centration | Tablet |
| No. | Ingredient | % w/w | (mg) |

Promethazine Layer

| | | | |
|---|---|---|---|
| 1 | Promethazine HCl | 3.57 | 12.5 |
| 2 | Croscarmellose Sodium (AcDiSol) | 1.90 | 6.667 |
| 3 | Croscarmellose Sodium (AcDiSol) | 2.38 | 8.333 |
| 4 | Silicified Microcrystalline | 9.29 | 32.5 |
| | Cellulose (Prosolv HD90) | | |
| 5 | Silicified Microcrystalline | 25.43 | 89 |
| | Cellulose (Prosolv HD90) | | |
| 6 | Magnesium Stearate | 0.29 | 1 |

Oxycodone Layer

| | | | |
|---|---|---|---|
| 7 | Oxycodone HCL | 1.43 | 5 |
| 8 | Croscarmellose Sodium (AcDiSol) | 1.9 | 6.667 |
| 9 | Hydroxypropyl methylcellulose | 1.6 | 5.6 |
| | (Methocel K4M) | | |
| 10 | Mannitol USP/NF (Pearlitol 300 DC) | 17.21 | 60.244 |
| 11 | Silicified Microcrystalline | 34.43 | 120.489 |
| | Cellulose (Prosolv HD90) | | |
| 12 | Magnesium Stearate | 0.29 | 1 |
| 13 | Stearic Acid | 0.29 | 1 |

| | | |
|---|---|---|
| Total | 100 | 350 |

TABLE 31
Promethazine layer batch

| | | Con- | Amount/ | Amount/ |
|---|---|---|---|---|
| Item | | centration | Tablet | Batch |
| No. | Ingredient | % w /w | (mg) | (g) |
| 1 | Promethazine HCl | 8.33 | 12.5 | 83.333 |
| 2 | Croscarmellose Sodium | 4.44 | 6.667 | 44.444 |
| | (AcDiSol) | | | |
| 3 | Croscarmellose Sodium | 5.56 | 8.333 | 55.556 |
| | (AcDiSol) | | | |
| 4 | Silicified Microcrystalline | 21.67 | 32.5 | 216.667 |
| | Cellulose (Prosolv HD90) | | | |
| 5 | Silicified Microcrystalline | 59.33 | 89.0 | 593.333 |
| | Cellulose (Prosolv HD90) | | | |
| 6 | Magnesium Stearate | 0.67 | 1.0 | 6.667 |

| | | | |
|---|---|---|---|
| Total | 100 | 150.0 | 1000.0 |

TABLE 32
Oxycodone layer batch

| | | Con- | Amount/ | Amount/ |
|---|---|---|---|---|
| Item | | centration | Tablet | Batch |
| No. | Ingredient | % w/w | (mg) | (g) |
| 7 | Oxycodone HCL | 2.5 | 5.0 | 25.0* |
| 8 | Croscarmellose Sodium | 3.33 | 6.667 | 33.335 |
| | (AcDiSol) | | | |
| 9 | Hydroxypropyl methylcellulose | 2.8 | 5.6 | 28.0 |
| | (Methocel K4M) | | | |
| 10 | Mannitol USP/NF | 30.12 | 60.244 | 301.22 |
| | (Pearlitol 300 DC) | | | |
| 11 | Silicified Microcrystalline | 60.24 | 120.489 | 602.445* |
| | Cellulose (Prosolv HD90) | | | |
| 12 | Magnesium Stearate | 0.5 | 1.0 | 5.0 |
| 13 | Stearic Acid | 0.5 | 1.0 | 5.0 |

| | | | |
|---|---|---|---|
| Total | 100 | 200.0 | 1000.0 |

### Example 12

### Dissolution Profile of Oxycodone/Promethazine Tablets

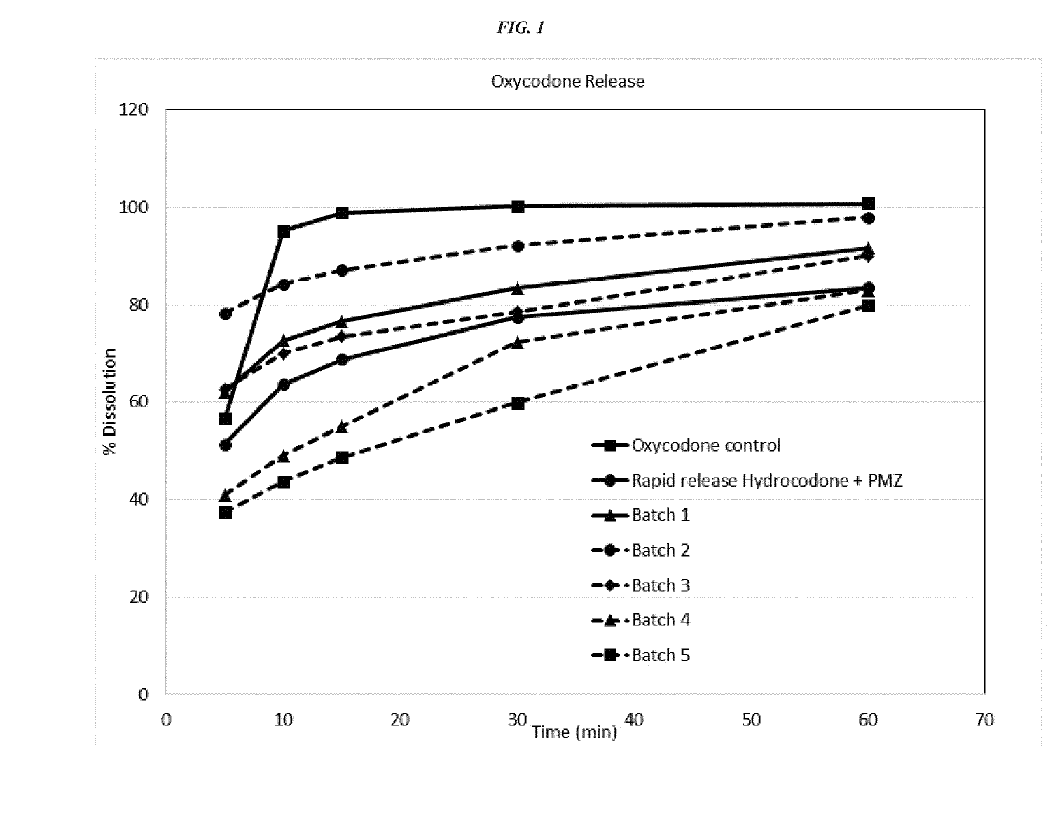

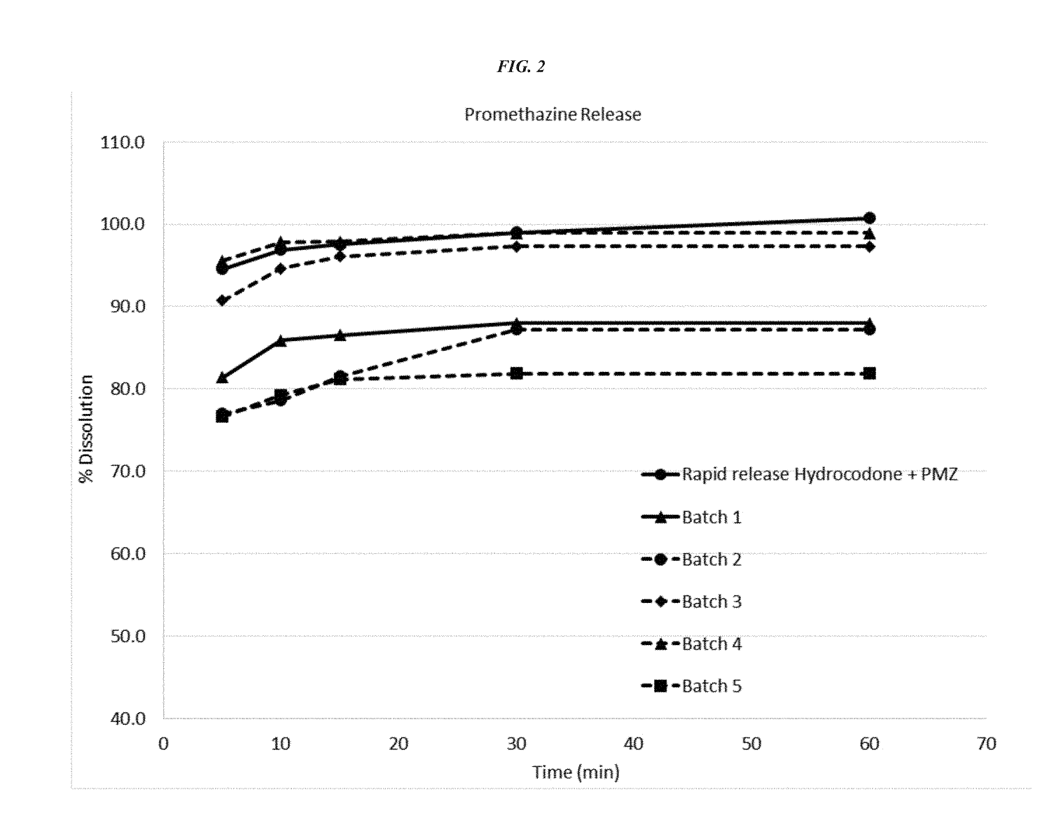

Oxycodone and Promethazine dissolution rates of the batch of tablets manufactured in Example 11 (Batch 3), as well as other manufactured batches (Batches 1, 2, 4, and 5) were determined and are summarized in Tables 33 and 34 and FIG. 1 and FIG. 2. The presented values for each batch are the average of six assays, where one tablet was assessed in each assay. As an oxycodone control was used, specifically a 5 mg tablet of Oxycodone Hydrochloride, brand name Roxicodone.

TABLE 33
Percent Oxycodone release

| | | Controlled | | | | | |
|---|---|---|---|---|---|---|---|
| | | Release | | | | | |
| | | Hydrocodone | | | | | |
| | Oxy- | in | | | | | |
| | codone | Composition | Batch | Batch | Batch | Batch | Batch |
| Time | control | 3 | 1 | 2 | 3 | 4 | 5 |
| 5 | 56.6 | 51.40764 | 62.07 | 78.3 | 62.69 | 40.92 | 37.37 |
| 10 | 95.1 | 63.64626 | 72.6 | 84.2 | 69.90 | 49.03 | 43.71 |
| 15 | 98.9 | 68.81828 | 76.54 | 87.1 | 73.43 | 54.99 | 48.69 |
| 30 | 100.2 | 77.39377 | 83.34 | 92.1 | 78.57 | 72.24 | 59.89 |
| 60 | 100.8 | 83.45473 | 91.6 | 97.9 | 90.02 | 82.94 | 79.85 |

TABLE 34
Percent Promethazine release

| | PMZ in | | | | | |
|---|---|---|---|---|---|---|
| Time | Composition 3 | Batch 1 | Batch 2 | Batch 3 | Batch 4 | Batch 5 |
| 5 | 94.6 | 81.39 | 77 | 90.72 | 95.55 | 76.62 |
| 10 | 96.9 | 85.9 | 78.6 | 94.60 | 97.84 | 79.27 |
| 15 | 97.5 | 86.53 | 81.5 | 96.05 | 97.90 | 81.16 |
| 30 | 99.0 | 87.97 | 87.2 | 97.29 | 98.94 | 81.88 |
| 60 | 100.8 | 88 | 87.2 | 97.29 | 98.94 | 81.88 |

### Example 13

### Dissolution Profile of Oxycodone/Promethazine Round and Oval Tablets

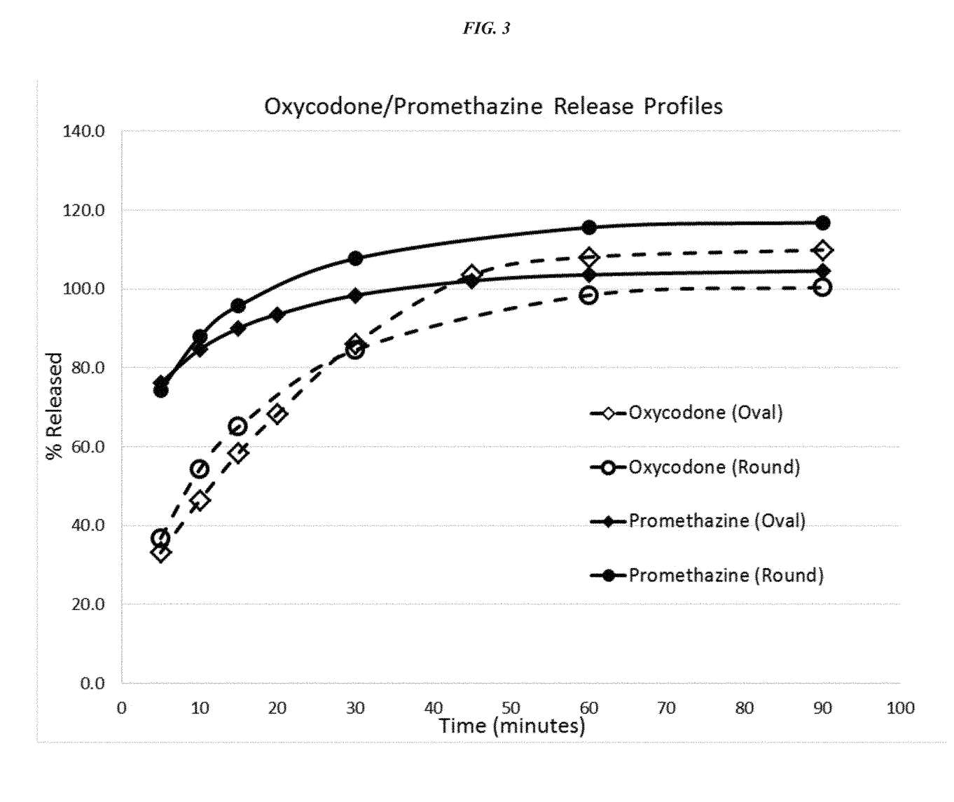

Oxycodone and Promethazine dissolution rates of a batch of tablets of formulation 1B of Table 3, manufactured in either an oval or round shape as indicated, were determined and are summarized in Table 35 and Table 36 and FIG. 3.

TABLE 35
Percent Oxycodone release

| Oval | | | Round | | |
|---|---|---|---|---|---|
| Time | % | Std. | Time | % | Std. |
| (min) | Release | Dev. | (min) | Release | Dev. |
| 5 | 33.1 | 5.7 | 5 | 36.76 | 9.2 |
| 10 | 46.3 | 5.6 | 10 | 54.2 | 14.4 |
| 15 | 58.3 | 5.6 | 15 | 65.03 | 15.7 |
| 20 | 68.3 | 6.4 | 30 | 84.50 | 11.0 |
| 30 | 86.2 | 9.1 | 60 | 98.4 | 2.5 |
| 45 | 103.5 | 5.6 | 90 | 100.4 | 2.2 |
| 60 | 108.1 | 1.1 | | | |
| 90 | 109.9 | 2.0 | | | |

TABLE 36
Percent Promethazine release

| Oval | | | Round | | |
|---|---|---|---|---|---|
| Time | % | Std. | Time | % | Std. |
| (min) | Release | Dev. | (min) | Release | Dev. |
| 5 | 76.2 | 3.7 | 5 | 74.33 | 9.8 |
| 10 | 84.6 | 3.7 | 10 | 87.8 | 9.5 |
| 15 | 90.0 | 4.0 | 15 | 95.72 | 8.0 |
| 20 | 93.5 | 4.0 | 30 | 107.68 | 5.9 |
| 30 | 98.4 | 4.2 | 60 | 115.6 | 1.9 |
| 45 | 102.0 | 3.1 | 90 | 116.8 | 1.4 |
| 60 | 103.6 | 2.2 | | | |
| 90 | 104.6 | 1.7 | | | |

### Example 14

### GMP Batch

1) Making of the Oxycodone HCl Blend with the Ingredients in Table 37:

Half of the starch, oxycodone HCl, and the other half of the starch were screened in order through a 20 mesh screen and then blended in a MAXIBLEND V-blender for 10 minutes to generate Blend 1. Half of the hydroxypropyl methylcellulose, croscarmellose sodium, Blend 1, the other half of the hydroxypropyl methylcellulose were screened in order through a 40 mesh screen and then blended in the V-blender for 10 minutes to generate Blend 2. Half of the microcrystalline cellulose, and mannitol were screened in order through a 20 mesh screen to produce Mix 1. Blend 2 and the other half of the microcrystalline cellulose were screened in order through a 40 mesh screen to produce Mix 2. Mix 1 and Mix 2 were blended in the V-blender for 20 minutes to generate Blend 3. The magnesium stearate and stearic acid were screened in order through a 40 mesh screen to produce Mix 3. Blend 3 and Mix 3 were blended in the V-blender for 5 minutes to generate the Oxycodone HCl Blend.

TABLE 37
Oxycodone HCL Blend Formula

| | | Con- | Amount/ |
|---|---|---|---|
| Item | | centration | Tablet |
| No. | Ingredient/Component No. | (% W/W) | (mg) |
| 1 | Oxycodone HCl, USP (OXY3 Fine) (CII) | 2.5 | 5.0 |
| 2 | Starch 1500 Partially Pregelatinized Maize | 10.0 | 20.0 |
| | Starch, NF | | |
| 3 | Hydroxypropyl Methylcellulose LISP, EP, JP | 5.17 | 10.34 |
| | (Methocel K4M Premium CR) | | |
| 4 | Ac-Di-Sol Croscarmellose Sodium, NF | 3.33 | 6.667 |
| | Ph.Eur, JP | | |
| 5 | Mannitol USP (Pearlitol 300 DC) | 19.33 | 38.667 |
| 6 | PROSOLV SMCC EID90 (Silicified | 58.67 | 117.33 |
| | Microcrystalline Cellulose, NF) | | |
| 7 | Magnesium Stearate, NF (Hyqual ® | 0.5 | 1.0 |
| | Vegetable Source) | | |

| | | |
|---|---|---|
| Total | 100 | 200.0 |

2) Making of the Promethazine HCl Blend with the Ingredients in Table 38:

The croscarmellose sodium 2A was added to a bag containing the promethazine HCl and mixed by hand for 1 minute, The mix was emptied from the bag and screened through a 20 mesh screen. The croscarmellose sodium 2B was added to the emptied bag to rinse the bag and then screened through a 20 mesh screen. The silicified microcrystalline cellulose 3A was also screened through a 20 mesh screen, The foregoing three groups of screened materials were blended in a MAXIBLEND V-blender for 10 minutes to produce Blend 1. Blend 1 and the silicified microcrystalline cellulose 3B was screened in order through a 20 mesh screen and then blended in the NT-blender for 20 minutes to produce Blend 2. The magnesium stearate was screened through a 40 mesh screen and blended with Blend 2 in the NT-blender for 5 minutes to generate the Promethazine HCl Blend.

TABLE 38
Promethazine HCl Blend Formula

| | | Con- | Amount/ |
|---|---|---|---|
| | Ingredient/Component | centration | Tablet |
| No. | No. | (% W/W) | (mg) |
| 1 | Promethazine HCl, USP | 8.33 | 12.5 |
| 2A | Ac-Di-Sol | 4.44 | 6.667 |
| 2B | Croscarmellose | 5.56 | 8.333 |
| | Sodium, NF Ph.Eur, JP | | |
| 3A | PROSOLV SMCC | 21.67 | 32.5 |
| | HD90 (Silicified | | |
| 3B | Microcrystalline | 59.33 | 89.0 |
| | Cellulose, NF) | | |
| 4 | Magnesium Stearate, | 0.67 | 1.0 |
| | NF (Hyqual ® | | |
| | Vegetable Source | | |

| | | |
|---|---|---|
| Total | 100 | 150.0 |

3) Making of the Bi-Layer Tablet in Table 39:

The Oxycodone HCl Blend was added to the hopper of the EP-200L Bi-layer Tablet Press for layer 1, and the press parameters were adjusted to achieve a 200 mg target weight. The Promethazine HCl Blend was added to the Press hopper for layer 2, and the parameters were adjusted as necessary to achieve a 150 mg target weight. The Press compressed the layers to achieve:

Tablet Layer 1 Weight: 200 mg±10 mg (190-210 mg)

Tablet Layer 2. Weight: 150 mg±7.5 mg (142.5-157.5 mg)

Tablet Total Weight: 350 mg±17.5 mg (332.5-367.5 mg)

Target Tablet Hardness: 10 kp

Average Tablet Hardness: 8 kp-12 kp

Individual Tablet Hardness: 6 kp-14 kp

Tablet Thickness: target=5.1 mm

TABLE 39
Bi-Layer Tablet of 5 mg Oxycodone HCl 12.5 mg Promethazine HCl

| | | | |
|---|---|---|---|
| | | Concen- | Amount/ |
| Item | | tration | Tablet |
| No. | Ingredient | % w/w | (mg) |

Promethazine Layer

| | | | |
|---|---|---|---|
| 1 | Promethazine HCl | 3.57 | 12.5 |
| 2 | Croscarmellose Sodium (AcDiSol) | 1.90 | 6.667 |
| 3 | Croscarmellose Sodium (AcDiSol) | 2.38 | 8.333 |
| 4 | Silicified Microcrystalline Cellulose | 9.29 | 32.5 |
| | (Prosolv SMCC HD90) | | |
| 5 | Silicified Microcrystalline Cellulose | 25.43 | 89 |
| | (Prosolv SMCC HD90) | | |
| 6 | Magnesium Stearate | 0.29 | 1 |

Oxycodone Layer

| | | | |
|---|---|---|---|
| 7 | Oxycodone HCL | 1.43 | 5 |
| 8 | Starch (1500 Partially | 5.71 | 20.0 |
| | Pregelatinized Maize Starch) | | |
| 9 | Hydroxypropyl methylcellulose | 2.95 | 10.34 |
| | Methocel K4M ) | | |
| 10 | Croscarmellose Sodium (AcDiSol) | 1.9 | 6.667 |
| 11 | Mannitol USP/NF (Pearlitol 300 DC) | 11.05 | 38.667 |
| 12 | Silicified Microcrystalline | 33.52 | 117.33 |
| | Cellulose (Prosolv HD90) | | |
| 13 | Magnesium Stearate | 0.29 | 1 |
| 14 | Stearic Acid | 0.29 | 1 |

| | | |
|---|---|---|
| Total | 100 | 350 |

4) Dissolution and Stability

Table 40 and Table 41 show the measurements of stability and dissolution of the tablets of Table 39 stored at 25-60° C. and 40-75° C. at 0, 1 month, and 2 months, respectively.

5) Dissolution Methods and Calculation

Dissolution apparatus was a USP Apparatus 1 (Basket). Dissolution fluid was 900 mL of de-aerated 0.01 N HCl, maintained at 37.0+/−0.5° C. during dissolution procedure. The fluid was prepared by diluting 12.5 mL of 12M HCl to 15 L with de-aerated water, and mixed. Standard Solution: 0.014 mg/mL, of promethazine HCl and 0.0055 mg/mL oxycodone HCl. To measure peaks, a dual wavelength detector (e.g., Hitachi L-2420) was used, or alternatively, two separate chromatographic systems can be used in order to measure the peaks at two different wavelengths.

A 50-μL aliquot was chromatographed on a 150-mm×4.6-mm (i.d.) Phenomenex Kinetex™ C₁₈, 2.6-μm particle size column using a gradient HPLC method. Mobile phase A consisted of water/KH₂PO₄ (monohydrate)/hepatanesulfonic sodium, 1000 g/2 g/1 g, pH 3.0±0.05, and mobile phase B consisted of 100% acetonitrile. The flow rate was 1.0 mL/minute. For example, the amount of oxycodone released was determined at 280 nm by comparing the area obtained for the peak due to oxycodone in the chromatogram of the dissolution test solution to that obtained for the corresponding peak in a chromatogram of a standard solution. The amount of promethazine released was determined at 280 nm by comparing the area obtained for the peak due to promethazine the chromatogram of the dissolution test solution to that obtained for the corresponding peak in a chromatogram of a standard solution.

Basket speed was 50 rpm; pull volume was 1.5 ml using an auto sampler; pull points: 5, 10, 15, 30, and 60 minutes, and 90 minutes spin out. The amount of each component dissolved in the dissolution medium was determined by HPLC. The method can use a high purity, bonded C18 stationary phase and a binary mobile phase consisting of an appropriate buffer and organic modifier.

Calculations

(a) Profile

$$\% Released = [ ( \frac{{R}_{u}}{{R}_{s}} \times {C}_{STD} \times {V}_{d} ) + {\sum }_{i = 1}^{n - 1} ( \frac{{R}_{i}}{{R}_{s}} \times {C}_{STD} \times {V}_{i} ) ] \times ( \frac{1}{LC} ) \times 100$$

Where:
   R_{U}=Area of Promethazine and Oxycodone in the Sample Solution
   R_{S}=Area of Promethazine and Oxycodone in all Standards
   C_{Std}=Concentration of Promethazine and Oxycodone in the Standard preparation (mg/mL)
   V_{d}=Volume of dissolution medium at the pull time (mL)
   Rᵢ=Peak area of Promethazine and Oxycodone obtained from the sample preparation at the individual pull points
   Vᵢ=Volume of the sample removed from the vessel at the previous pull point (mL)
   LC=Label Claim (mg)

While particular instances described herein have been shown and described herein, such instances are provided by way of example only. Numerous variations, changes, and substitutions can now occur to those skilled in the art without departing from what is disclosed herein. It should be understood that various alternatives to the instances described herein can be employed in practicing embodiments disclosed herein. It is intended that the following claims define the scope of some instances of the invention and that methods and structures within the scope of these claims and their equivalents be covered thereby.

What is claimed is:

1. A solid oral pharmaceutical composition, wherein the solid oral pharmaceutical composition comprises:
a. a first matrix, wherein the first matrix comprises:
i. about 0.5 mg to 30 mg of an opioid analgesic,
ii. about 1 mg to 15 mg of croscarmellose sodium,
iii. about 1 mg to 20 mg of hydroxypropyl methylcellulose,
iv. about 10 mg to 100 mg of mannitol,
v. about 50 mg to 200 mg of silicified microcrystalline cellulose,
vi. about 0.1 mg to 5 mg of magnesium stearate, and
vii. about 0.1 mg to 5 mg of stearic acid; and
b. a second matrix, wherein the second matrix comprises:
i. about 5 mg to 30 mg of an antiemetic,
ii. about 100 mg to 250 mg of silicified microcrystalline cellulose,
iii. about 5 mg to 25 mg of croscarmellose sodium, and
iv. about 0.1 mg to 5 mg of magnesium stearate.

2. The solid oral pharmaceutical composition of claim 1, wherein the opioid analgesic is selected from the group consisting of oxycodone, hydrocodone, tramadol, hydromorphone, alfentanil, buprenorphine, butorphanol, codeine, codeine, dezocine, diamorphine, fentanyl, levacetylmethadol, levorphanol, meperidine, methadone, morphine, nalbuphine, oxymorphone, pentazocine, propoxyphene, remifentanil, sufentanil, a pharmaceutically acceptable salt thereof, and any combination thereof.

3. The solid oral pharmaceutical composition of claim 2, wherein the opioid analgesic is oxycodone or a pharmaceutically acceptable salt thereof.

4. The solid oral pharmaceutical composition of claim 3, wherein the opioid analgesic is oxycodone hydrochloride.

5. The solid oral pharmaceutical composition of claim 1, wherein the opioid analgesic is present in an amount of about 2.5 mg, about 5 mg, about 7.5 mg, about 10 mg, about 15 mg, or about 20 mg.

6. The solid oral pharmaceutical composition of claim 1, wherein the antiemetic is promethazine or a pharmaceutically acceptable salt thereof.

7. The solid oral pharmaceutical composition of claim 6, wherein the antiemetic is promethazine hydrochloride.

8. The solid oral pharmaceutical composition of claim 1, wherein the antiemetic is present in an amount of about 12.5 to 25 mg.

9. The solid oral pharmaceutical composition of claim 1, wherein the first matrix further comprises about 5 to 50 mg of starch.

10. The solid oral pharmaceutical composition of claim 9, wherein in the first matrix,
a. the opioid analgesic is oxycodone hydrochloride that is present in an amount of about 5 mg,
b. the starch is present in an amount of about 20 mg,
c. the croscarmellose sodium is present in an amount of about 6.7 mg,
d. the hydroxypropyl methylcellulose is present in an amount of about 10.3 mg,
e. the mannitol is present in an amount of about 38.7 mg,
f. the silicified microcrystalline cellulose is present in an amount of about 117.3 mg,
g. the magnesium stearate is present in an amount of about 1 mg, and
h. the stearic acid is present in an amount of about 1 mg.

11. The solid oral pharmaceutical composition of claim 1, wherein in the first matrix,
a. the opioid analgesic is oxycodone hydrochloride that is present in an amount of about 5 mg,
b. the croscarmellose sodium is present in an amount of about 6.7 mg,
c. the hydroxypropyl methylcellulose is present in an amount of about 5.6 mg,
d. the mannitol is present in an amount of about 60.2 mg,
e. the silicified microcrystalline cellulose is present in an amount of about 120.5 mg,
f. the magnesium stearate is present in an amount of about 1 mg, and
g. the stearic acid is present in an amount of about 1 mg.

12. The solid oral pharmaceutical composition of claim 1, wherein in the second matrix,
a. the antiemetic is promethazine hydrochloride that is present in an amount of about 12.5 mg,
b. the silicified microcrystalline cellulose is present in an amount of about 121.5 mg,
c. the croscarmellose sodium is present in an amount of about 15 mg, and
d. the magnesium stearate is present in an amount of about 1 mg.

13-16. (canceled)

17. The solid oral pharmaceutical composition of claim 1, wherein the first matrix is a layer.

18. The solid oral pharmaceutical composition of claim 1, wherein the second matrix is a layer.

19. The solid oral pharmaceutical composition of claim 1, wherein the solid oral pharmaceutical composition provides an effective amount of the opioid analgesic to treat or prevent pain in a subject in need thereof and an effective amount of the antiemetic to reduce or prevent an adverse effect associated with the opioid analgesic in the subject.

20. A solid oral pharmaceutical composition, wherein the solid oral pharmaceutical composition comprises:
a. a first matrix, wherein the first matrix comprises:
i. an opioid analgesic,
ii. croscarmellose sodium,
iii. hydroxypropyl methylcellulose,
iv. mannitol,
v. silicified microcrystalline cellulose,
vi. magnesium stearate, and
vii. stearic acid; and
b. a second matrix, wherein the second matrix comprises:
i. an antiemetic,
ii. silicified microcrystalline cellulose,
iii. croscarmellose sodium, and
iv. magnesium stearate,
wherein: when the solid oral pharmaceutical composition is stored at a temperature less than 80° C. for a time period of at least 30 days, about 90% to about 100% of the antiemetic is active as measured by HPLC, and about 80% to about 100% of the opioid analgesic is active for as measured by HPLC.

21-35. (canceled)

36. A solid oral pharmaceutical composition, wherein the solid oral pharmaceutical composition comprises:
a first matrix that comprises an opioid analgesic; and
a second matrix that comprises an antiemetic,
wherein about 30% to about 60% of the antiemetic is released within about 5 to 15 minutes in a dissolution medium as measured by USP Apparatus 1 with a basket rotating at 50 rpm, wherein the dissolution medium is 900 mL of 0.01 N HCl at about 37° C.

37-55. (canceled)

56. A method for treating or preventing pain, comprising orally administering to a subject in need thereof a solid oral pharmaceutical composition that comprises:
a. a first matrix, wherein the first matrix comprises:
i. about 0.5 mg to 30 mg of an opioid analgesic,
ii. about 1 mg to 15 mg of croscarmellose sodium,
iii. about 1 mg to 20 mg of hydroxypropyl methylcellulose,
iv. about 10 mg to 100 mg of mannitol,
v. about 50 mg to 200 mg of silicified microcrystalline cellulose,
vi. about 0.1 mg to 5 mg of magnesium stearate, and
vii. about 0.1 mg to 5 mg of stearic acid; and
b. a second matrix, wherein the second matrix comprises:
i. about 5 mg to 30 mg of an antiemetic,
ii. about 100 mg to 250 mg of silicified microcrystalline cellulose,
iii. about 5 mg to 25 mg of croscarmellose sodium, and
iv. about 0.1 mg to 5 mg of magnesium stearate.

57-61. (canceled)

62. The method of claim 56, wherein the solid oral pharmaceutical composition is administered at least two times a day.

63. (canceled)

64. The method of claim 56, wherein the solid oral pharmaceutical composition is administered in a dosing interval of about 4 to about 6 hours.

65-72. (canceled)
